(19) United States
(12) Patent Application Publication
(10) Pub. No.: US 2022/0024927 A1
(43) Pub. Date: Jan. 27, 2022

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Chang Ju SHIN, Suwon-si (KR); Dong Wan RYU, Suwon-si (KR); Jongwoo WON, Suwon-si (KR); Dongkyu RYU, Suwon-si (KR); Hanill LEE, Suwon-si (KR); Jinseok JANG, Suwon-si (KR); Sung-Hyun JUNG, Suwon-si (KR); Ho Kuk JUNG, Suwon-si (KR)

(21) Appl. No.: 17/315,910

(22) Filed: May 10, 2021

(30) Foreign Application Priority Data
May 11, 2020 (KR) 10-2020-0056026

(51) Int. Cl. C07D 487/00; C07D 493/00; C07D 495/00; H01L 51/00

(52) U.S. Cl. CPC C07D 487/00; C07D 493/00; C07D 495/00; H01L 51/0074; H01L 51/0072; H01L 51/0073; H01L 51/0067

(57) ABSTRACT

A compound for an organic optoelectronic device, a composition for an organic optoelectronic device, an organic optoelectronic device, and a display device, the compound being represented by Chemical Formula 1:

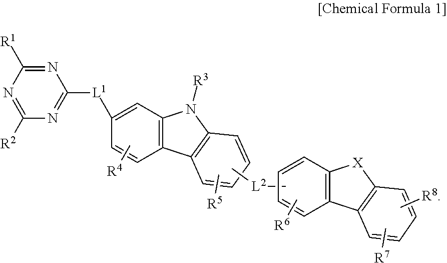

### CROSS-REFERENCE TO RELATED APPLICATION

Korean Patent Application No. 10-2020-0056026, filed on May 11, 2020, in the Korean Intellectual Property Office, and entitled: “Compound for Organic Optoelectronic Device, Composition for Organic Optoelectronic Device, Organic Optoelectronic Device, and Display Device,” is incorporated by reference herein in its entirety.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

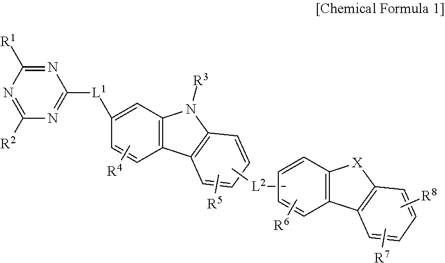

wherein, in Chemical Formula 1, X is O or S, L¹ and L² are independently a single bond or a substituted or unsubstituted C6 to C20 arylene group, R¹ to R³ are independently a substituted or unsubstituted C6 to C30 aryl group or a substituted or unsubstituted C2 to C30 heterocyclic group, and R⁴ to R⁸ are independently hydrogen, deuterium, a cyano group, a halogen, a substituted or unsubstituted C1 to C30 alkyl group, or a substituted or unsubstituted C6 to C30 aryl group.

The embodiments may be realized by providing a composition for an organic optoelectronic device, the composition including a first compound for an organic optoelectronic device and a second compound for an organic optoelectronic device, wherein the first compound is the compound according to an embodiment, and the second compound is represented by Chemical Formula 2; or a combination of Chemical Formula 3 and Chemical Formula 4:

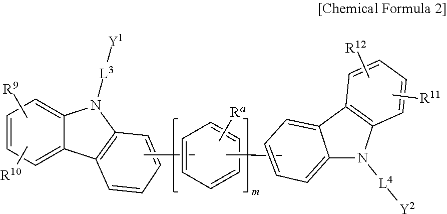

in Chemical Formula 2, Y¹ and Y² are independently a substituted or unsubstituted C6 to C20 aryl group, or a substituted or unsubstituted C2 to C30 heterocyclic group, L³ and L⁴ are independently a single bond or a substituted or unsubstituted C6 to C20 arylene group, R^{a} and R⁹ to R¹² are independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, and m is an integer of 0 to 2;

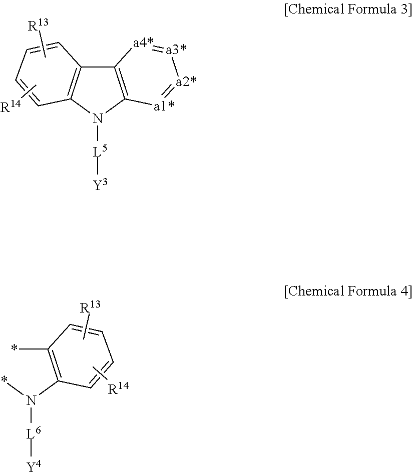

in Chemical Formulas 3 and 4, Y³ and Y⁴ are independently a substituted or unsubstituted C6 to C20 aryl group or a substituted or unsubstituted C2 to C30 heterocyclic group, an adjacent two of a1* to a4* are linking carbons linked at * of Chemical Formula 4 and the other two of a1* to a4* are C-L^{a}-R^{b}, L^{a}, L⁵, and L⁶ are independently a single bond or a substituted or unsubstituted C6 to C20 arylene group, and R^{b} and R¹³ to R¹⁶ are independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group.

The embodiments may be realized by providing an organic optoelectronic device including an anode and a cathode facing each other, and at least one organic layer between the anode and the cathode, wherein the at least one organic layer includes the compound or the composition according to an embodiment.

The embodiments may be realized by providing a display device including the organic optoelectronic device according to an embodiment.

### BRIEF DESCRIPTION OF THE DRAWINGS

Features will be apparent to those of skill in the art by describing in detail exemplary embodiments with reference to the attached drawings in which:

FIGS. 1 and 2 are cross-sectional views of an organic light emitting diode according to embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof. As used herein, the term “or” is not an exclusive term, e.g., “A or B” would include A, B, or A and B.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, or a combination thereof.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an embodiment is described.

The compound for an organic optoelectronic device according to an embodiment may be represented by Chemical Formula 1.

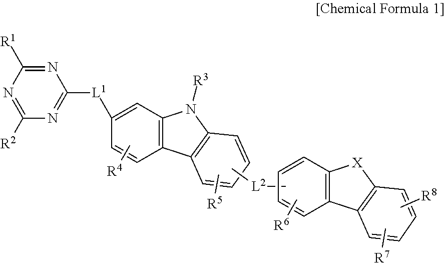

In Chemical Formula 1, X may be, e.g., O or S.

L¹ and L² may each independently be or include, e.g., a single bond or a substituted or unsubstituted C6 to C20 arylene group.

R¹ to R³ may each independently be or include, e.g., a substituted or unsubstituted C6 to C30 aryl group or a substituted or unsubstituted C2 to C30 heterocyclic group.

R⁴ to R⁸ may each independently be or include, e.g., hydrogen, deuterium, a cyano group, a halogen, a substituted or unsubstituted C1 to C30 alkyl group, or a substituted or unsubstituted C6 to C30 aryl group.

The compound represented by Chemical Formula 1 may include a carbazole core wherein a phenyl moiety in one direction or at one side of the carbazole core is substituted with a triazine group, and a phenyl moiety in the other direction or at another side is substituted with a dibenzofuranyl group, or a dibenzothiophenyl group.

In an implementation, by simultaneously introducing a triazine group and a dibenzofuranyl group (or dibenzothiophenyl group) on the phenyl moieties of the carbazole core, movement speeds of holes and electrons may increase and thereby a carrier balance may effectively increase in light emitting layer, obtaining an organic optoelectronic device with a higher life-span.

In an implementation, the triazine group may be bonded at the number 2 position of the carbazole core, and the electron transfer effect may be further improved.

In an implementation, by including a dibenzofuranyl group (or a dibenzothiophenyl group), the HOMO electron cloud in the molecule may be further enlarged compared with that substituted with an N-carbazolyl group to help enhance hole transport characteristics, thereby further improving the effect of reducing a driving voltage and the device life-span in the light emitting layer.

In an implementation, the compound represented by Chemical Formula 1 may be represented by one of Chemical Formula 1A to Chemical Formula 1D, e.g., depending on the specific substitution position at which the phenyl moiety of the carbazole core is substituted with the dibenzofuranyl group (or dibenzothiophenyl group).

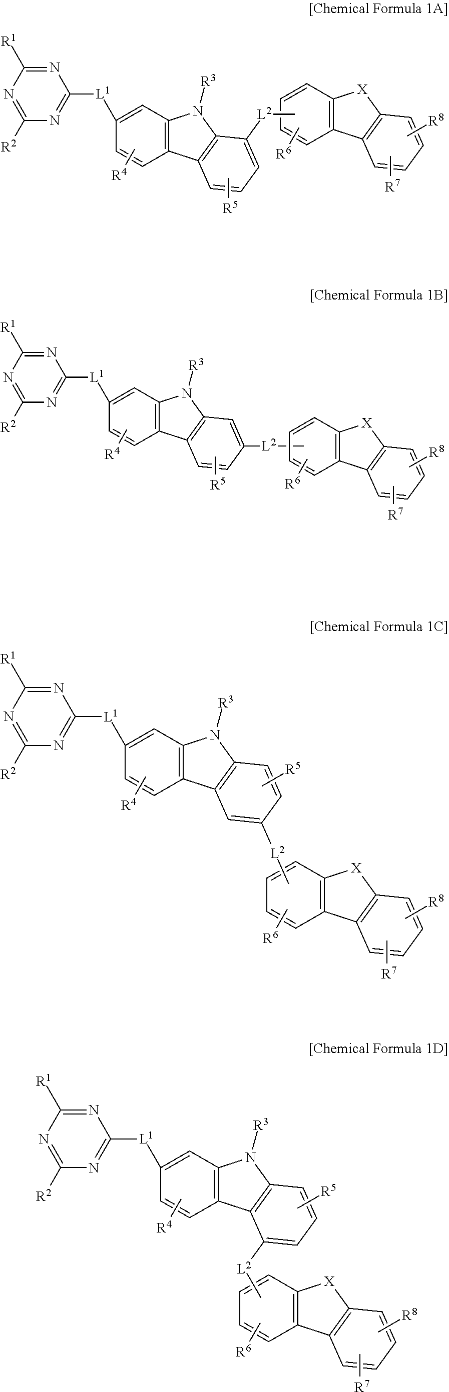

In Chemical Formula 1A to Chemical Formula 1D, X, L¹, L², and R¹ to R⁸ may be defined the same as described above.

In an implementation, the compound represented by Chemical Formula 1A may be represented by one of Chemical Formulae 1A-1 to 1A-4 depending on each point at which a dibenzofuranyl group (or dibenzothiophenyl group) is linked to the carbazole core.

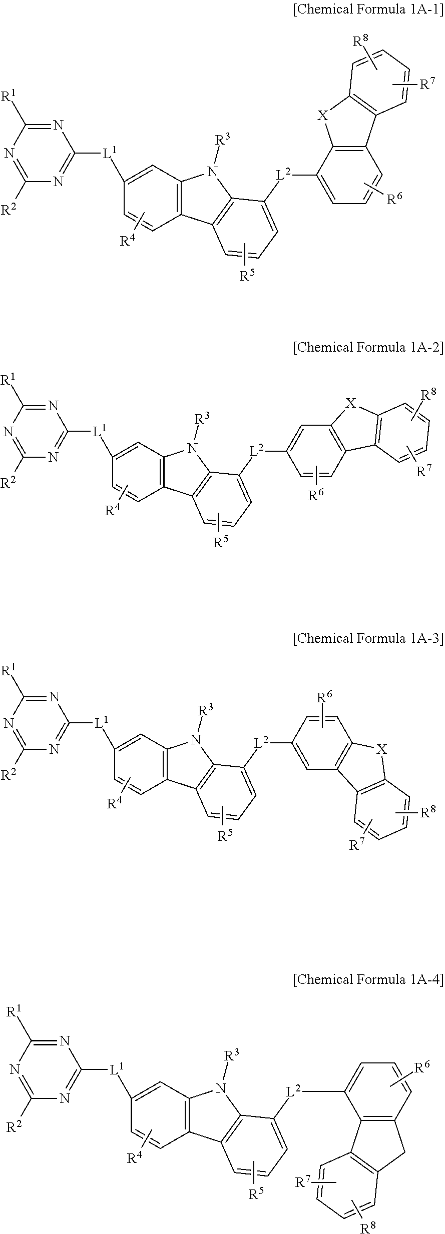

In an implementation, the compound represented by Chemical Formula 1B may be represented by one of Chemical Formulae 1B-1 to 1B-4 depending on the point at which a dibenzofuranyl group (or dibenzothiophenyl group) is linked to the carbazole core.

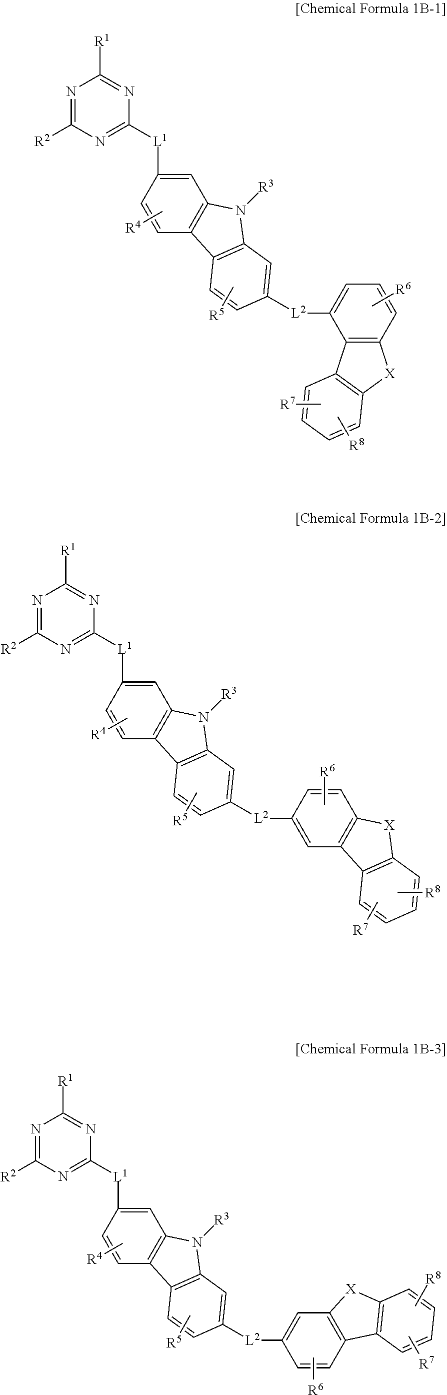

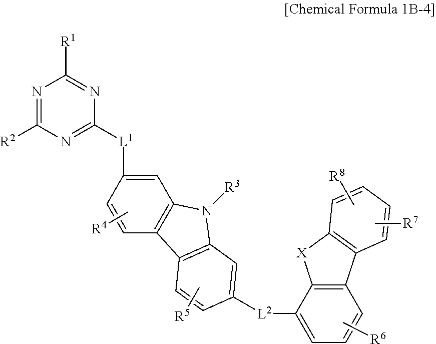

In an implementation, the compound represented by Chemical Formula 1C may be represented by one of Chemical Formula 1C-1 to Chemical Formula 1C-4 depending on the point at which a dibenzofuranyl group (or dibenzothiophenyl group) is linked to the carbazole core.

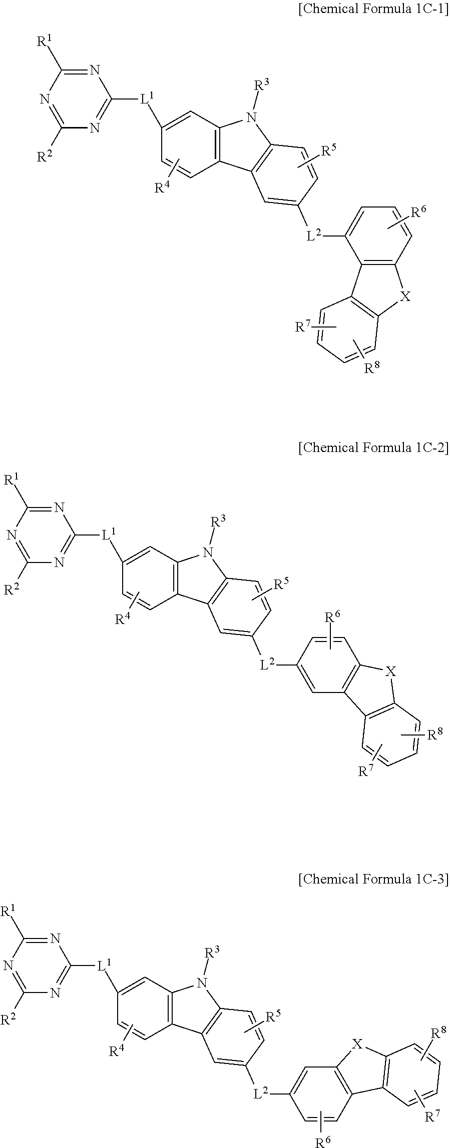

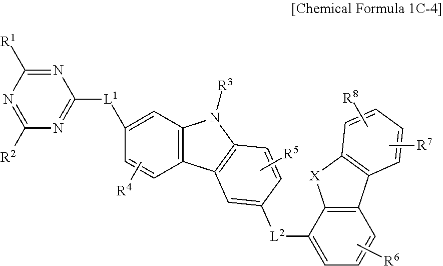

In an implementation, the compound represented by Chemical Formula 1D may be represented by one of Chemical Formula 1D-1 to Chemical Formula 1D-4 depending on the point at which a dibenzofuranyl group (or dibenzothiophenyl group) is linked to the carbazole core.

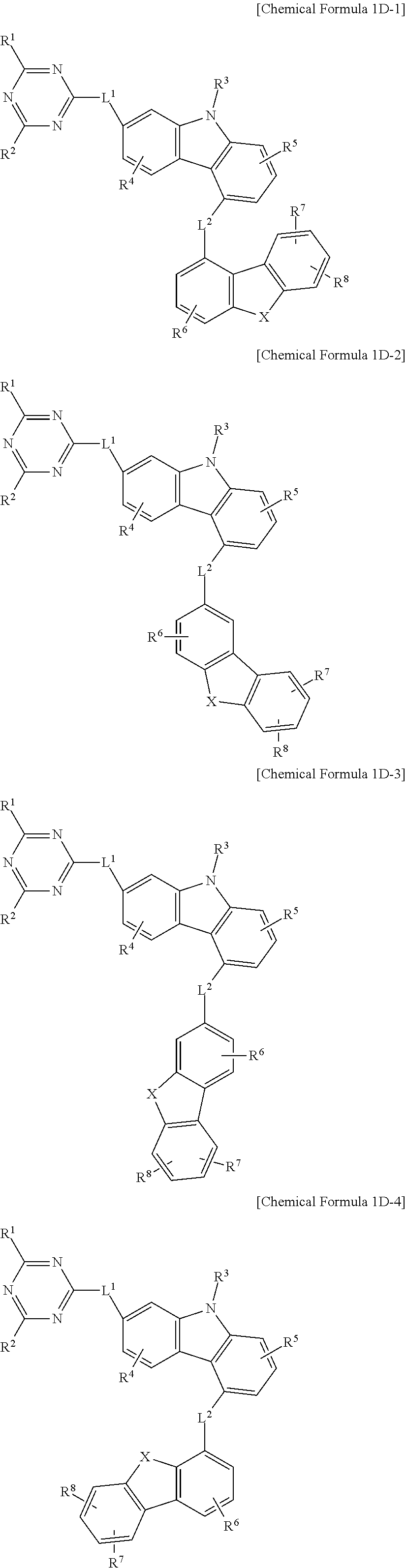

In Chemical Formula 1A-1 to Chemical Formula 1A-4, Chemical Formula 1B-1 to Chemical Formula 1B-4, Chemical Formula 1C-1 to Chemical Formula 1C-4, and Chemical Formula 1D-1 to Chemical Formula 1D-4, X, L¹, L², and R¹ to R⁸ may be defined the same as described above.

In an implementation, the compound represented by Chemical Formula 1 may be represented by Chemical Formula 1C.

In an implementation, the compound represented by Chemical Formula 1 may be represented by Chemical Formula 1C-2.

In an implementation, R¹ and R² may independently be, e.g., a substituted or unsubstituted phenyl group, a substituted or unsubstituted para-biphenyl group, or a substituted or unsubstituted meta-biphenyl group.

In an implementation, R³ may be, e.g., a substituted or unsubstituted phenyl group.

In an implementation, R⁴ to R⁸ may independently be, e.g., hydrogen, deuterium, a substituted or unsubstituted C1 to C5 alkyl group, or a substituted or unsubstituted C6 to C12 aryl group.

In an implementation, R⁴ to R⁸ may independently be, e.g., hydrogen or a substituted or unsubstituted phenyl group.

In an implementation, L¹ and L² may each be, e.g., a single bond.

In an implementation, the compound represented by Chemical Formula 1 may be a compound of the following Group 1.

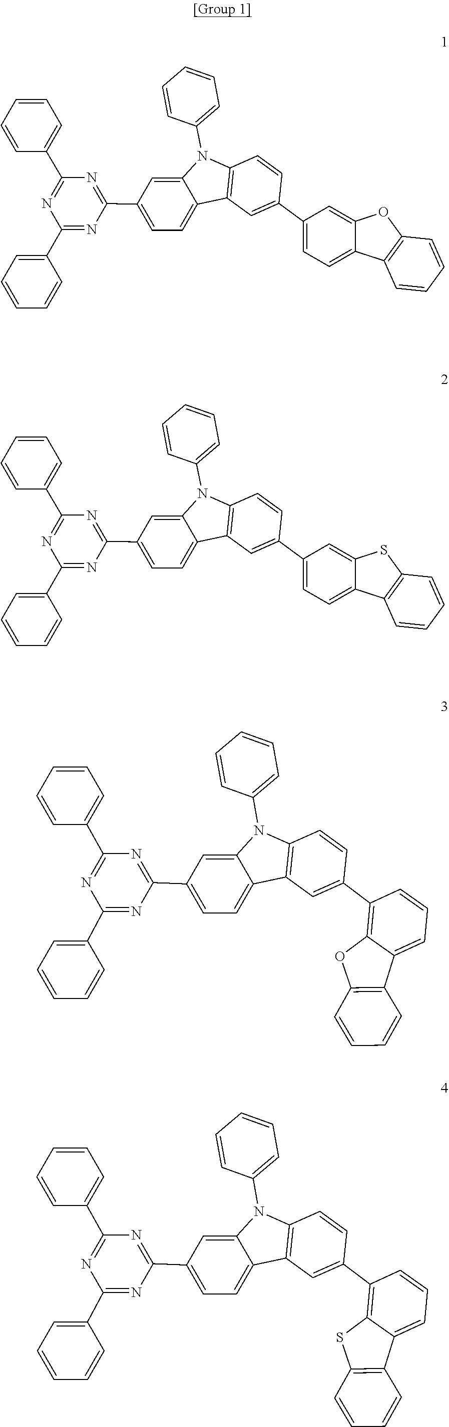 [SMILES: c1ccc(-c2nc(-c3ccccc3)nc(-c3ccc4c5cc(-c6ccc7c(c6)oc6ccccc67)ccc5n(-c5ccccc5)c4c3)n2)cc1 ; c1ccc(-c2nc(-c3ccccc3)nc(-c3ccc4c5cc(-c6ccc7c(c6)sc6ccccc67)ccc5n(-c5ccccc5)c4c3)n2)cc1 ; c1ccc(-c2nc(-c3ccccc3)nc(-c3ccc4c5cc(-c6cccc7c6oc6ccccc67)ccc5n(-c5ccccc5)c4c3)n2)cc1 ; c1ccc(-c2nc(-c3ccccc3)nc(-c3ccc4c5cc(-c6cccc7c6sc6ccccc67)ccc5n(-c5ccccc5)c4c3)n2)cc1]

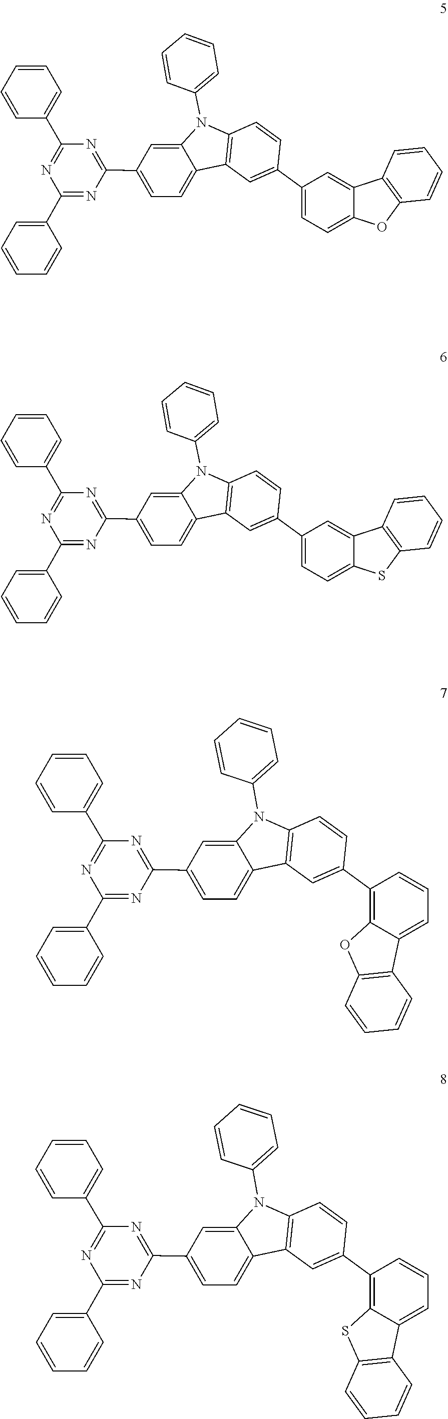 [SMILES: c1ccc(-c2nc(-c3ccccc3)nc(-c3ccc4c5cc(-c6ccc7oc8ccccc8c7c6)ccc5n(-c5ccccc5)c4c3)n2)cc1 ; c1ccc(-c2nc(-c3ccccc3)nc(-c3ccc4c5cc(-c6ccc7sc8ccccc8c7c6)ccc5n(-c5ccccc5)c4c3)n2)cc1 ; c1ccc(-c2nc(-c3ccccc3)nc(-c3ccc4c5cc(-c6cccc7c6oc6ccccc67)ccc5n(-c5ccccc5)c4c3)n2)cc1 ; c1ccc(-c2nc(-c3ccccc3)nc(-c3ccc4c5cc(-c6cccc7c6sc6ccccc67)ccc5n(-c5ccccc5)c4c3)n2)cc1]

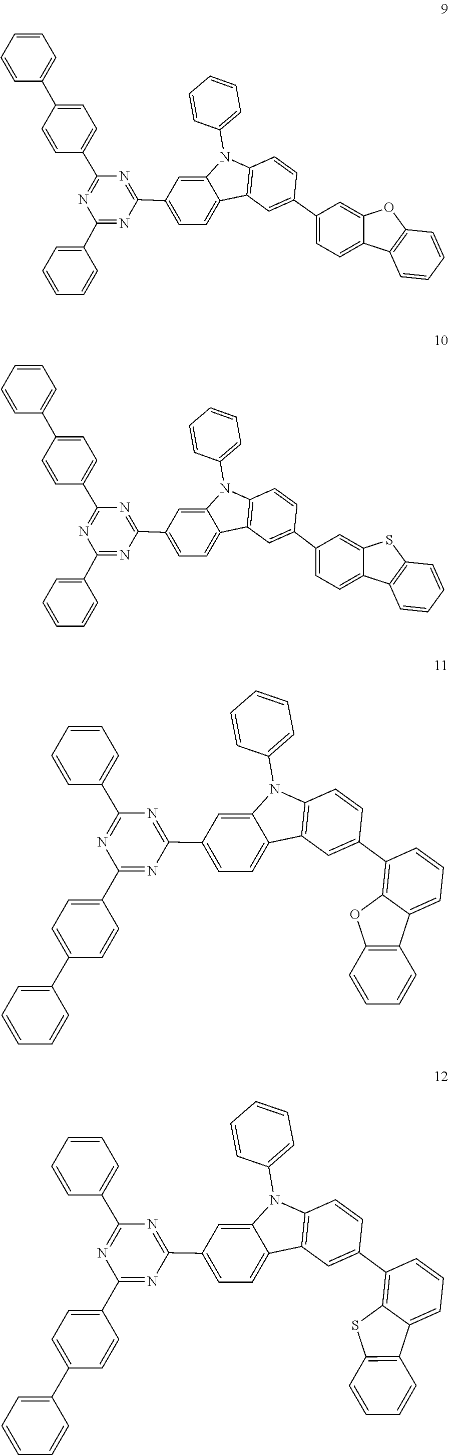 [SMILES: c1ccc(-c2ccc(-c3nc(-c4ccccc4)nc(-c4ccc5c6cc(-c7ccc8c(c7)oc7ccccc78)ccc6n(-c6ccccc6)c5c4)n3)cc2)cc1 ; c1ccc(-c2ccc(-c3nc(-c4ccccc4)nc(-c4ccc5c6cc(-c7ccc8c(c7)sc7ccccc78)ccc6n(-c6ccccc6)c5c4)n3)cc2)cc1 ; c1ccc(-c2ccc(-c3nc(-c4ccccc4)nc(-c4ccc5c6cc(-c7cccc8c7oc7ccccc78)ccc6n(-c6ccccc6)c5c4)n3)cc2)cc1 ; c1ccc(-c2ccc(-c3nc(-c4ccccc4)nc(-c4ccc5c6cc(-c7cccc8c7sc7ccccc78)ccc6n(-c6ccccc6)c5c4)n3)cc2)cc1]

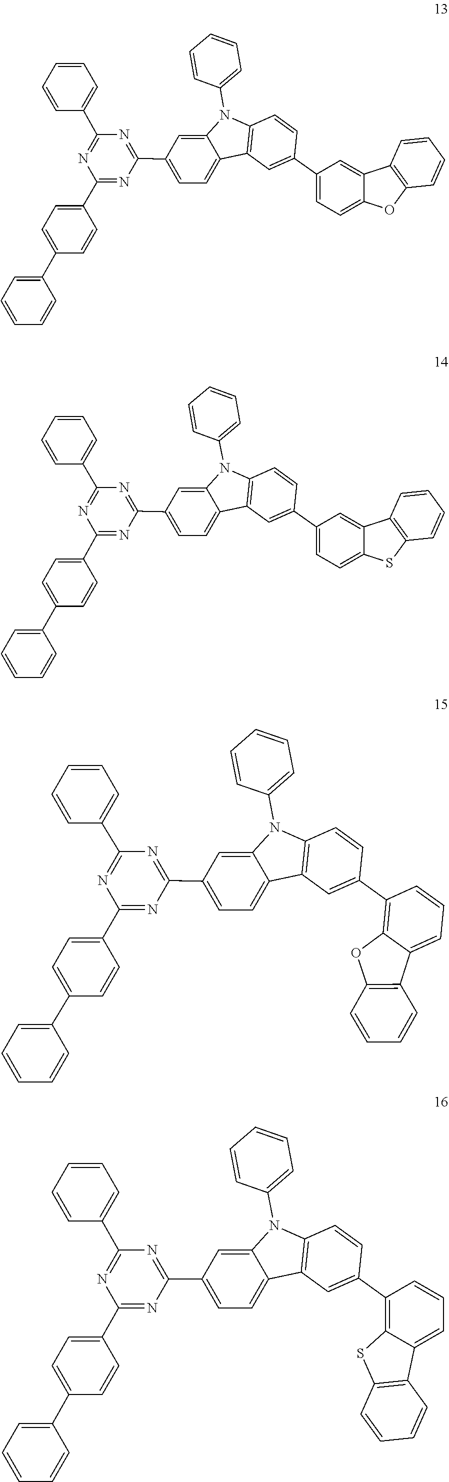 [SMILES: c1ccc(-c2ccc(-c3nc(-c4ccccc4)nc(-c4ccc5c6cc(-c7ccc8oc9ccccc9c8c7)ccc6n(-c6ccccc6)c5c4)n3)cc2)cc1 ; c1ccc(-c2ccc(-c3nc(-c4ccccc4)nc(-c4ccc5c6cc(-c7ccc8sc9ccccc9c8c7)ccc6n(-c6ccccc6)c5c4)n3)cc2)cc1 ; c1ccc(-c2ccc(-c3nc(-c4ccccc4)nc(-c4ccc5c6cc(-c7cccc8c7oc7ccccc78)ccc6n(-c6ccccc6)c5c4)n3)cc2)cc1 ; c1ccc(-c2ccc(-c3nc(-c4ccccc4)nc(-c4ccc5c6cc(-c7cccc8c7sc7ccccc78)ccc6n(-c6ccccc6)c5c4)n3)cc2)cc1]

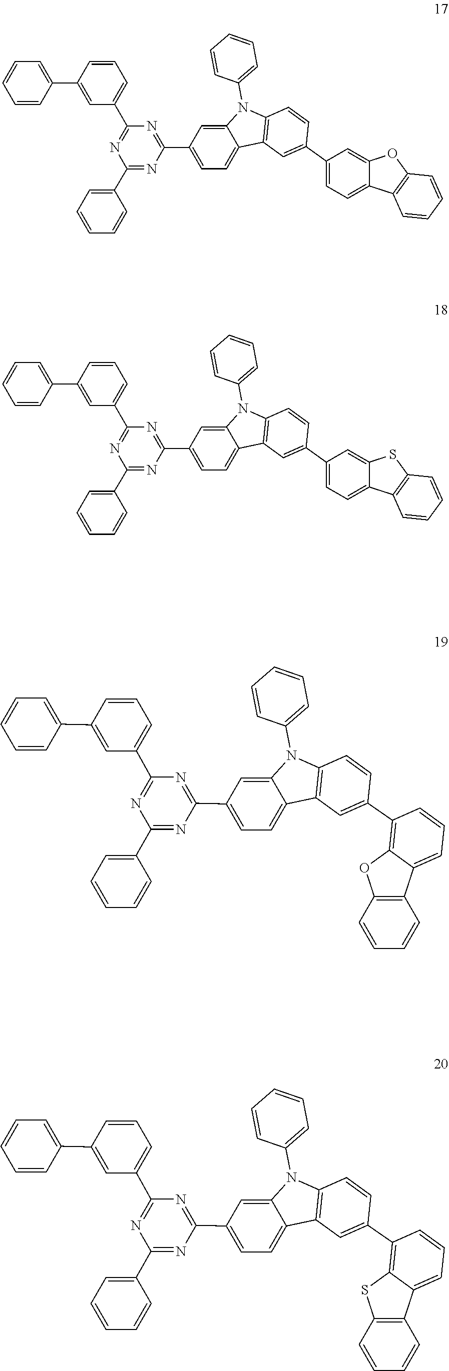 [SMILES: c1ccc(-c2cccc(-c3nc(-c4ccccc4)nc(-c4ccc5c6cc(-c7ccc8c(c7)oc7ccccc78)ccc6n(-c6ccccc6)c5c4)n3)c2)cc1 ; c1ccc(-c2cccc(-c3nc(-c4ccccc4)nc(-c4ccc5c6cc(-c7ccc8c(c7)sc7ccccc78)ccc6n(-c6ccccc6)c5c4)n3)c2)cc1 ; c1ccc(-c2cccc(-c3nc(-c4ccccc4)nc(-c4ccc5c6cc(-c7cccc8c7oc7ccccc78)ccc6n(-c6ccccc6)c5c4)n3)c2)cc1 ; c1ccc(-c2cccc(-c3nc(-c4ccccc4)nc(-c4ccc5c6cc(-c7cccc8c7sc7ccccc78)ccc6n(-c6ccccc6)c5c4)n3)c2)cc1]

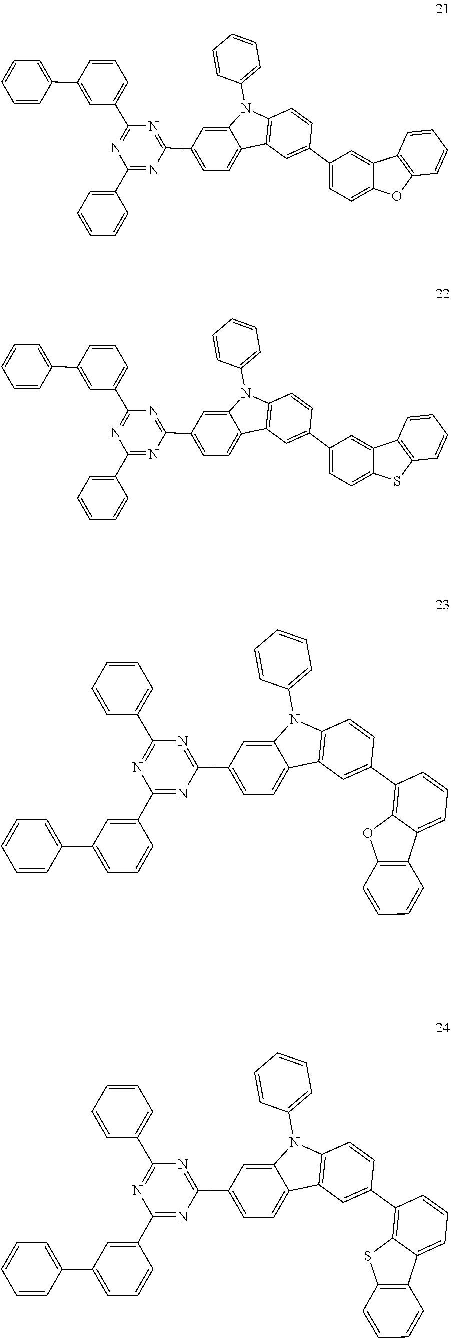 [SMILES: c1ccc(-c2cccc(-c3nc(-c4ccccc4)nc(-c4ccc5c6cc(-c7ccc8oc9ccccc9c8c7)ccc6n(-c6ccccc6)c5c4)n3)c2)cc1 ; c1ccc(-c2cccc(-c3nc(-c4ccccc4)nc(-c4ccc5c6cc(-c7ccc8sc9ccccc9c8c7)ccc6n(-c6ccccc6)c5c4)n3)c2)cc1 ; c1ccc(-c2cccc(-c3nc(-c4ccccc4)nc(-c4ccc5c6cc(-c7cccc8c7oc7ccccc78)ccc6n(-c6ccccc6)c5c4)n3)c2)cc1 ; c1ccc(-c2cccc(-c3nc(-c4ccccc4)nc(-c4ccc5c6cc(-c7cccc8c7sc7ccccc78)ccc6n(-c6ccccc6)c5c4)n3)c2)cc1]

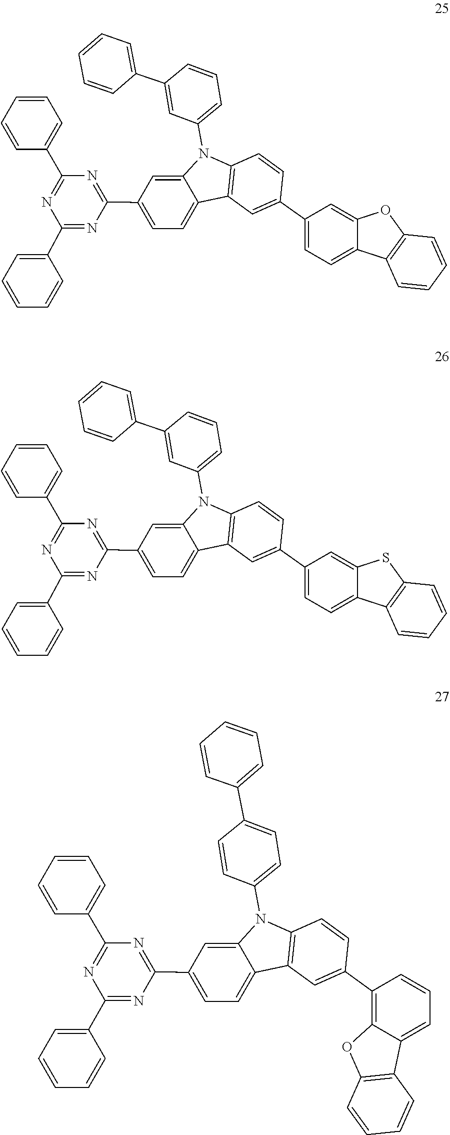 [SMILES: c1ccc(-c2cccc(-n3c4ccc(-c5ccc6c(c5)oc5ccccc56)cc4c4ccc(-c5nc(-c6ccccc6)nc(-c6ccccc6)n5)cc43)c2)cc1 ; c1ccc(-c2cccc(-n3c4ccc(-c5ccc6c(c5)sc5ccccc56)cc4c4ccc(-c5nc(-c6ccccc6)nc(-c6ccccc6)n5)cc43)c2)cc1 ; c1ccc(-c2ccc(-n3c4ccc(-c5cccc6c5oc5ccccc56)cc4c4ccc(-c5nc(-c6ccccc6)nc(-c6ccccc6)n5)cc43)cc2)cc1]

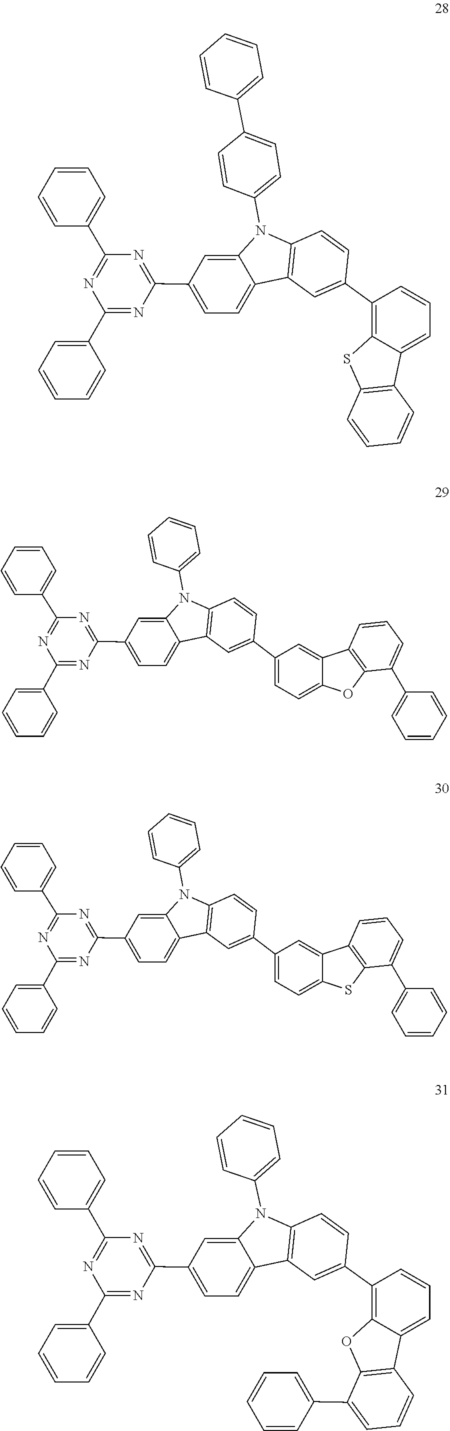 [SMILES: c1ccc(-c2ccc(-n3c4ccc(-c5cccc6c5sc5ccccc56)cc4c4ccc(-c5nc(-c6ccccc6)nc(-c6ccccc6)n5)cc43)cc2)cc1 ; c1ccc(-c2nc(-c3ccccc3)nc(-c3ccc4c5cc(-c6ccc7oc8c(-c9ccccc9)cccc8c7c6)ccc5n(-c5ccccc5)c4c3)n2)cc1 ; c1ccc(-c2nc(-c3ccccc3)nc(-c3ccc4c5cc(-c6ccc7sc8c(-c9ccccc9)cccc8c7c6)ccc5n(-c5ccccc5)c4c3)n2)cc1 ; c1ccc(-c2nc(-c3ccccc3)nc(-c3ccc4c5cc(-c6cccc7c6oc6c(-c8ccccc8)cccc67)ccc5n(-c5ccccc5)c4c3)n2)cc1]

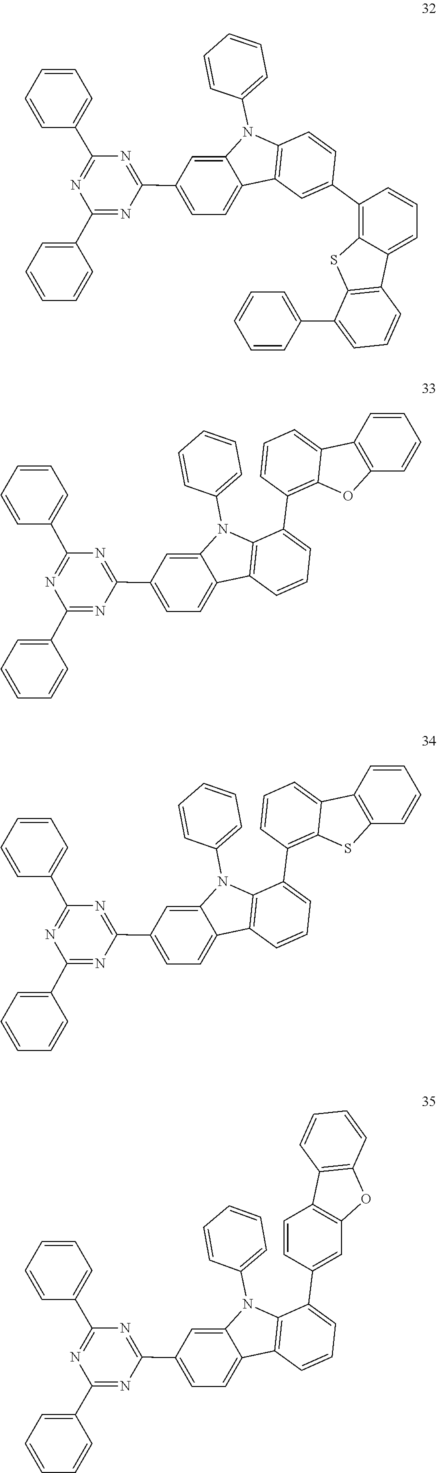 [SMILES: c1ccc(-c2nc(-c3ccccc3)nc(-c3ccc4c5cc(-c6cccc7c6sc6c(-c8ccccc8)cccc67)ccc5n(-c5ccccc5)c4c3)n2)cc1 ; c1ccc(-c2nc(-c3ccccc3)nc(-c3ccc4c5cccc(-c6cccc7c6oc6ccccc67)c5n(-c5ccccc5)c4c3)n2)cc1 ; c1ccc(-c2nc(-c3ccccc3)nc(-c3ccc4c5cccc(-c6cccc7c6sc6ccccc67)c5n(-c5ccccc5)c4c3)n2)cc1 ; c1ccc(-c2nc(-c3ccccc3)nc(-c3ccc4c5cccc(-c6ccc7c(c6)oc6ccccc67)c5n(-c5ccccc5)c4c3)n2)cc1]

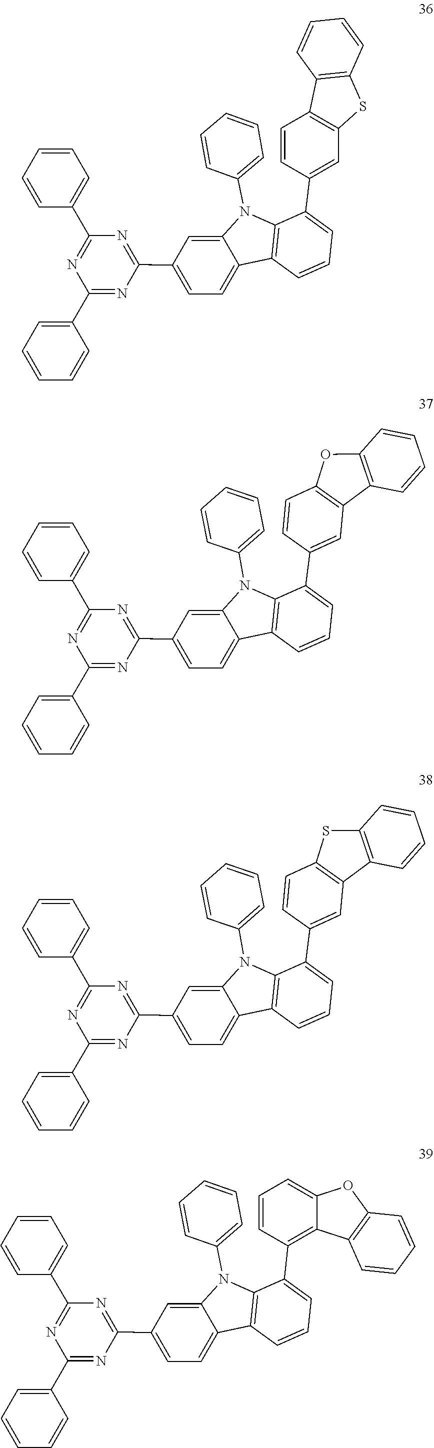 [SMILES: c1ccc(-c2nc(-c3ccccc3)nc(-c3ccc4c5cccc(-c6ccc7c(c6)sc6ccccc67)c5n(-c5ccccc5)c4c3)n2)cc1 ; c1ccc(-c2nc(-c3ccccc3)nc(-c3ccc4c5cccc(-c6ccc7oc8ccccc8c7c6)c5n(-c5ccccc5)c4c3)n2)cc1 ; c1ccc(-c2nc(-c3ccccc3)nc(-c3ccc4c5cccc(-c6ccc7sc8ccccc8c7c6)c5n(-c5ccccc5)c4c3)n2)cc1 ; c1ccc(-c2nc(-c3ccccc3)nc(-c3ccc4c5cccc(-c6cccc7oc8ccccc8c67)c5n(-c5ccccc5)c4c3)n2)cc1]

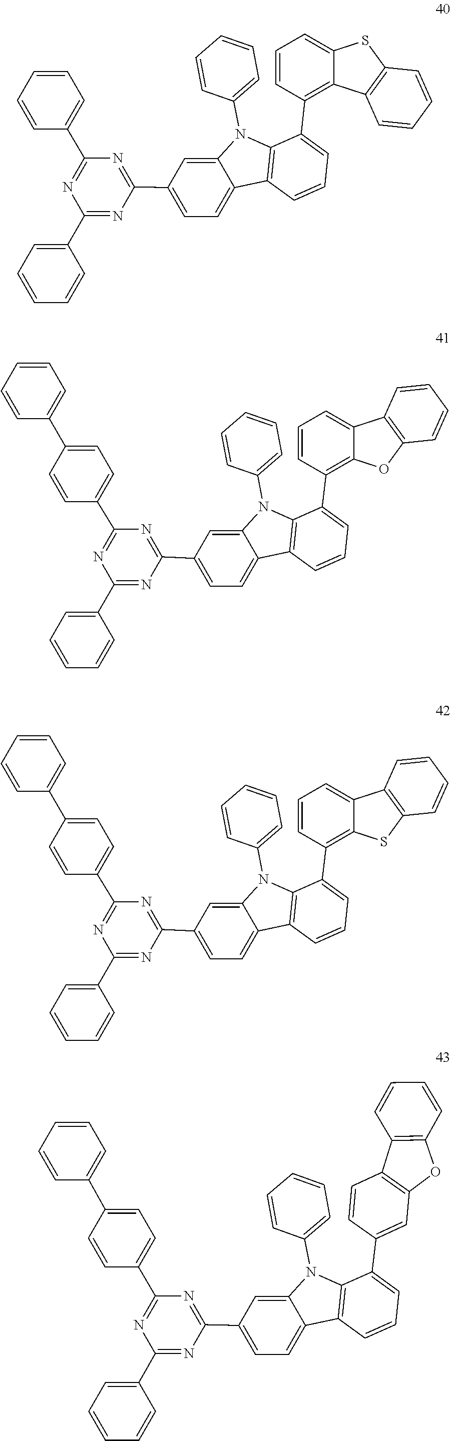 [SMILES: c1ccc(-c2nc(-c3ccccc3)nc(-c3ccc4c5cccc(-c6cccc7sc8ccccc8c67)c5n(-c5ccccc5)c4c3)n2)cc1 ; c1ccc(-c2ccc(-c3nc(-c4ccccc4)nc(-c4ccc5c6cccc(-c7cccc8c7oc7ccccc78)c6n(-c6ccccc6)c5c4)n3)cc2)cc1 ; c1ccc(-c2ccc(-c3nc(-c4ccccc4)nc(-c4ccc5c6cccc(-c7cccc8c7sc7ccccc78)c6n(-c6ccccc6)c5c4)n3)cc2)cc1 ; c1ccc(-c2ccc(-c3nc(-c4ccccc4)nc(-c4ccc5c6cccc(-c7ccc8c(c7)oc7ccccc78)c6n(-c6ccccc6)c5c4)n3)cc2)cc1]

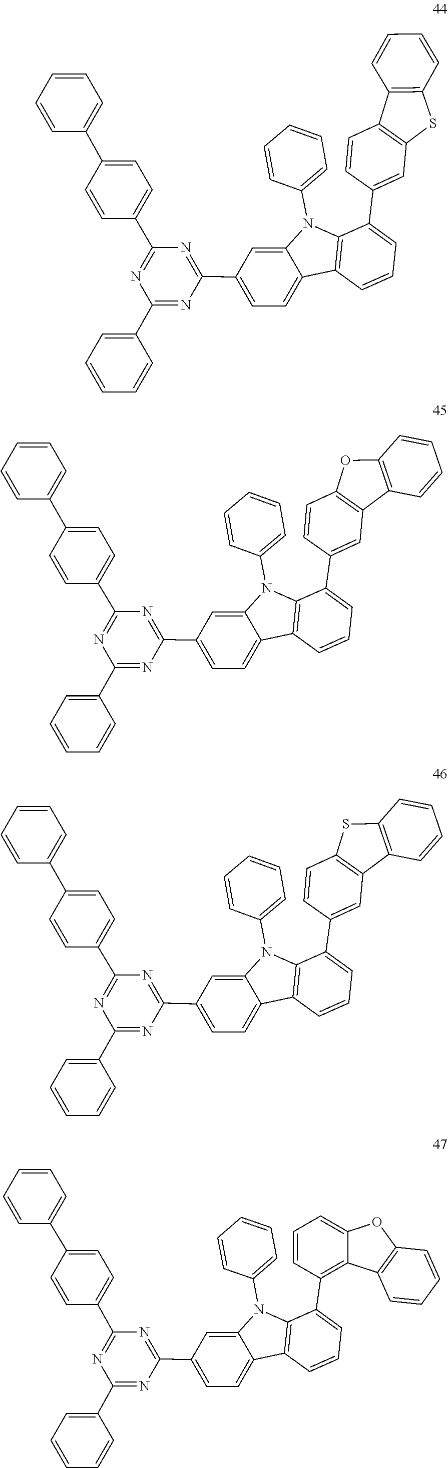 [SMILES: c1ccc(-c2ccc(-c3nc(-c4ccccc4)nc(-c4ccc5c6cccc(-c7ccc8c(c7)sc7ccccc78)c6n(-c6ccccc6)c5c4)n3)cc2)cc1 ; c1ccc(-c2ccc(-c3nc(-c4ccccc4)nc(-c4ccc5c6cccc(-c7ccc8oc9ccccc9c8c7)c6n(-c6ccccc6)c5c4)n3)cc2)cc1 ; c1ccc(-c2ccc(-c3nc(-c4ccccc4)nc(-c4ccc5c6cccc(-c7ccc8sc9ccccc9c8c7)c6n(-c6ccccc6)c5c4)n3)cc2)cc1 ; c1ccc(-c2ccc(-c3nc(-c4ccccc4)nc(-c4ccc5c6cccc(-c7cccc8oc9ccccc9c78)c6n(-c6ccccc6)c5c4)n3)cc2)cc1]

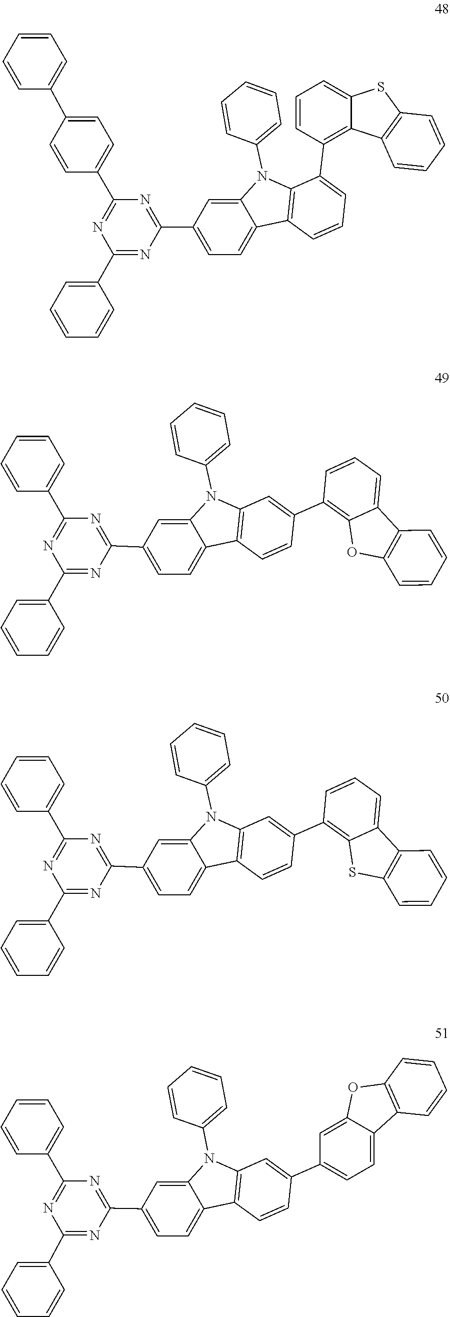 [SMILES: c1ccc(-c2ccc(-c3nc(-c4ccccc4)nc(-c4ccc5c6cccc(-c7cccc8sc9ccccc9c78)c6n(-c6ccccc6)c5c4)n3)cc2)cc1 ; c1ccc(-c2nc(-c3ccccc3)nc(-c3ccc4c5ccc(-c6cccc7c6oc6ccccc67)cc5n(-c5ccccc5)c4c3)n2)cc1 ; c1ccc(-c2nc(-c3ccccc3)nc(-c3ccc4c5ccc(-c6cccc7c6sc6ccccc67)cc5n(-c5ccccc5)c4c3)n2)cc1 ; c1ccc(-c2nc(-c3ccccc3)nc(-c3ccc4c5ccc(-c6ccc7c(c6)oc6ccccc67)cc5n(-c5ccccc5)c4c3)n2)cc1]

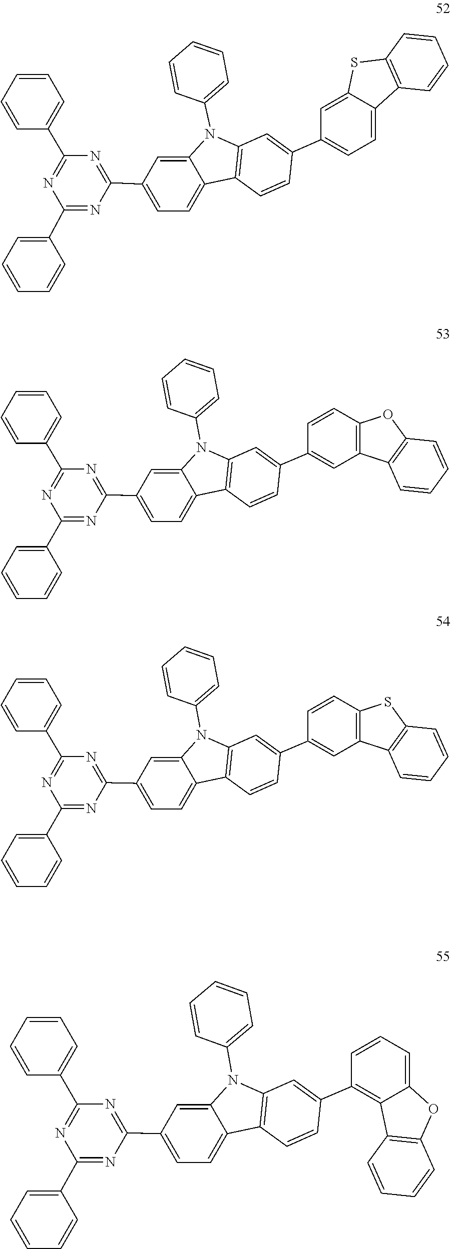 [SMILES: c1ccc(-c2nc(-c3ccccc3)nc(-c3ccc4c5ccc(-c6ccc7c(c6)sc6ccccc67)cc5n(-c5ccccc5)c4c3)n2)cc1 ; c1ccc(-c2nc(-c3ccccc3)nc(-c3ccc4c5ccc(-c6ccc7oc8ccccc8c7c6)cc5n(-c5ccccc5)c4c3)n2)cc1 ; c1ccc(-c2nc(-c3ccccc3)nc(-c3ccc4c5ccc(-c6ccc7sc8ccccc8c7c6)cc5n(-c5ccccc5)c4c3)n2)cc1 ; c1ccc(-c2nc(-c3ccccc3)nc(-c3ccc4c5ccc(-c6cccc7oc8ccccc8c67)cc5n(-c5ccccc5)c4c3)n2)cc1]

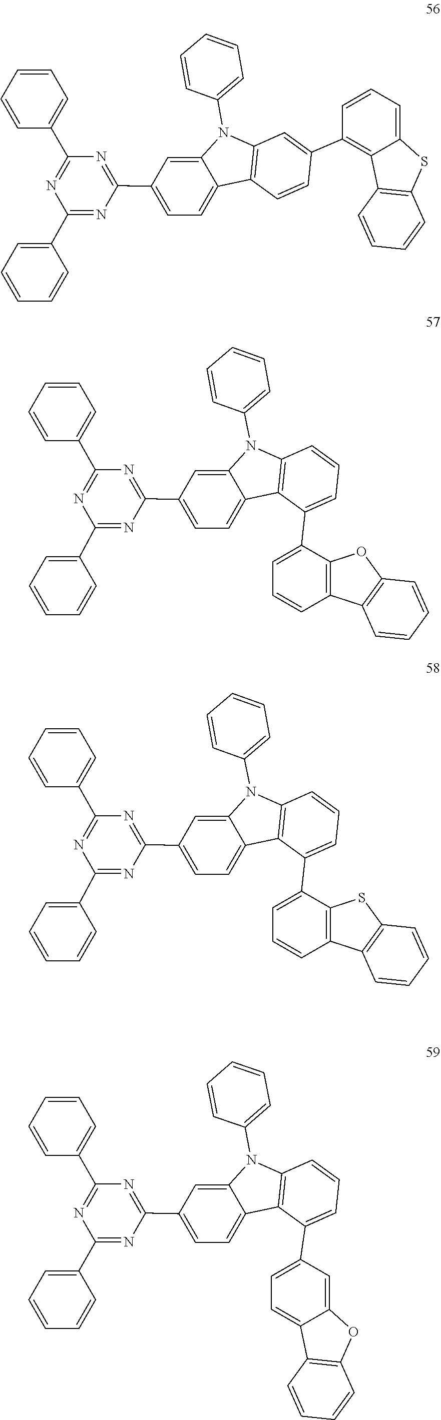 [SMILES: c1ccc(-c2nc(-c3ccccc3)nc(-c3ccc4c5ccc(-c6cccc7sc8ccccc8c67)cc5n(-c5ccccc5)c4c3)n2)cc1 ; c1ccc(-c2nc(-c3ccccc3)nc(-c3ccc4c5c(-c6cccc7c6oc6ccccc67)cccc5n(-c5ccccc5)c4c3)n2)cc1 ; c1ccc(-c2nc(-c3ccccc3)nc(-c3ccc4c5c(-c6cccc7c6sc6ccccc67)cccc5n(-c5ccccc5)c4c3)n2)cc1 ; c1ccc(-c2nc(-c3ccccc3)nc(-c3ccc4c5c(-c6ccc7c(c6)oc6ccccc67)cccc5n(-c5ccccc5)c4c3)n2)cc1]

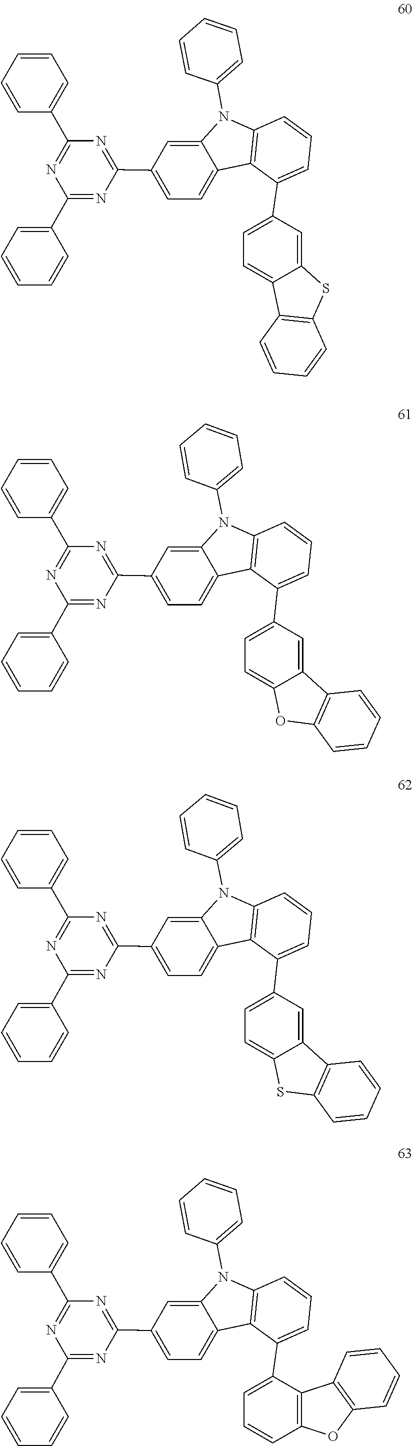 [SMILES: c1ccc(-c2nc(-c3ccccc3)nc(-c3ccc4c5c(-c6ccc7c(c6)sc6ccccc67)cccc5n(-c5ccccc5)c4c3)n2)cc1 ; c1ccc(-c2nc(-c3ccccc3)nc(-c3ccc4c5c(-c6ccc7oc8ccccc8c7c6)cccc5n(-c5ccccc5)c4c3)n2)cc1 ; c1ccc(-c2nc(-c3ccccc3)nc(-c3ccc4c5c(-c6ccc7sc8ccccc8c7c6)cccc5n(-c5ccccc5)c4c3)n2)cc1 ; c1ccc(-c2nc(-c3ccccc3)nc(-c3ccc4c5c(-c6cccc7oc8ccccc8c67)cccc5n(-c5ccccc5)c4c3)n2)cc1]

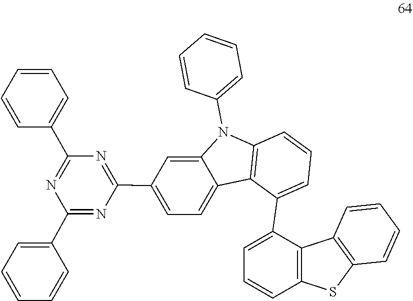 [SMILES: c1ccc(-c2nc(-c3ccccc3)nc(-c3ccc4c5c(-c6cccc7sc8ccccc8c67)cccc5n(-c5ccccc5)c4c3)n2)cc1]

In an implementation, the aforementioned compound for an organic optoelectronic device may be applied in the form of a composition.

In an implementation, the aforementioned compound for an organic optoelectronic device may be applied in the form of a composition further including another suitable compound.

In an implementation, the composition for an organic optoelectronic device may include a first compound for an organic optoelectronic device and a second compound for an organic optoelectronic device. The first compound may be the aforementioned compound (represented by Chemical Formula 1), and the second compound may be represented by Chemical Formula 2; or represented by a combination of Chemical Formula 3 and Chemical Formula 4.

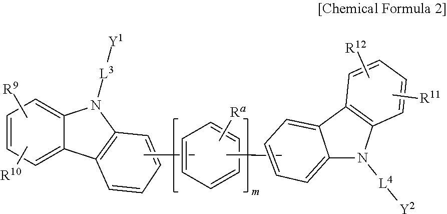

In Chemical Formula 2, ¹ and Y² may each independently be or include, e.g., a substituted or unsubstituted C6 to C20 aryl group or a substituted or unsubstituted C2 to C30 heterocyclic group.

L³ and L⁴ may each independently be or include, e.g., a single bond or a substituted or unsubstituted C6 to C20 arylene group,

R^{a} and R⁹ to R¹² may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group.

m may be, e.g., an integer of 0 to 2;

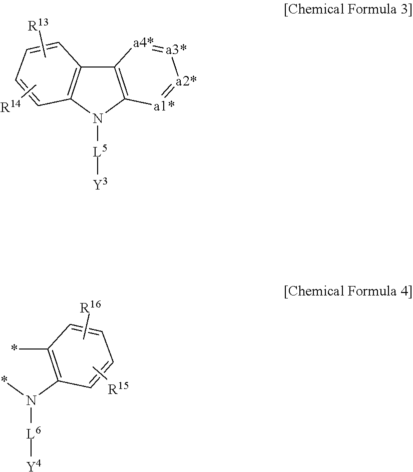

In Chemical Formulae 3 and 4, Y³ and Y⁴ may each independently be or include, e.g., a substituted or unsubstituted C6 to C20 aryl group or a substituted or unsubstituted C2 to C30 heterocyclic group.

a1* to a4* may each independently be, e.g., a linking carbon or C-L^{a}-R^{b}. In an implementation, adjacent two of a1* to a4* may be linking carbons linked to or at * of Chemical Formula 4. As used herein, the term “linking carbon” refers to a shared carbon at which fused rings are linked.

L^{a}, L⁵, and L⁶ may each independently be or include, e.g., a single bond or a substituted or unsubstituted C6 to C20 arylene group.

R^{b} and R¹³ to R¹⁶ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group.

The second compound may be used or included in a light emitting layer together (e.g., mixed) with the first compound for an organic optoelectronic device to help increase charge mobility and stability, thereby improving luminous efficiency and life-span characteristics.

In an implementation, Y¹ and Y² of Chemical Formula 2 may independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, a substituted or unsubstituted fluorenyl group, or a substituted or unsubstituted pyridinyl group.

In an implementation, L³ and L⁴ of Chemical Formula 2 may independently be, e.g., a single bond, a substituted or unsubstituted phenylene group, or a substituted or unsubstituted biphenylene group.

In an implementation, R^{a} and R⁹ to R¹² of Chemical Formula 2 may independently be, e.g., hydrogen, deuterium, or a substituted or unsubstituted C6 to C12 aryl group.

m may be, e.g., 0 or 1.

The “substituted” of Chemical Formula 2 may refer to replacement of at least one hydrogen by deuterium, a C1 to C4 alkyl group, a C6 to C18 aryl group, or a C2 to C30 heteroaryl group.

In an implementation, the compound represented by Chemical Formula 2 may be represented by one of Chemical Formula 2-1 to Chemical Formula 2-15.

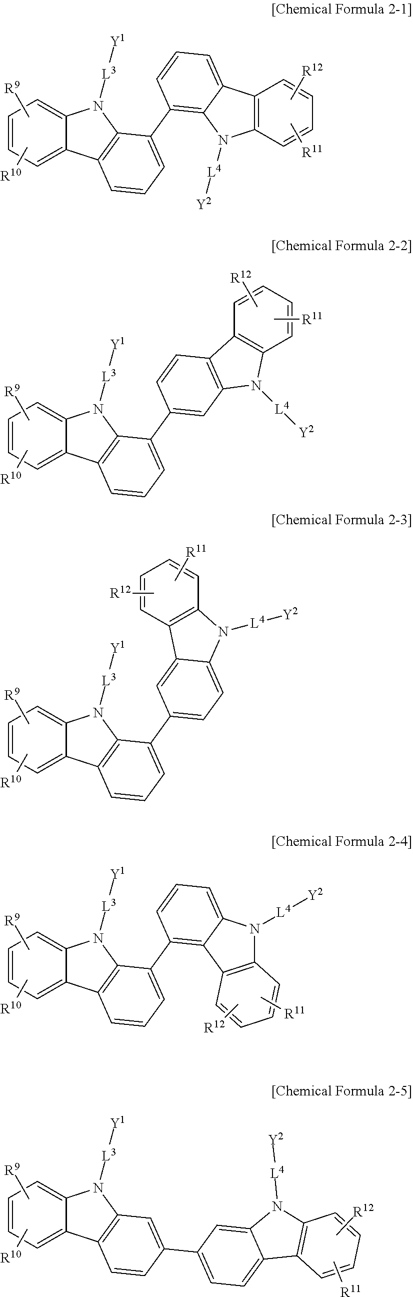

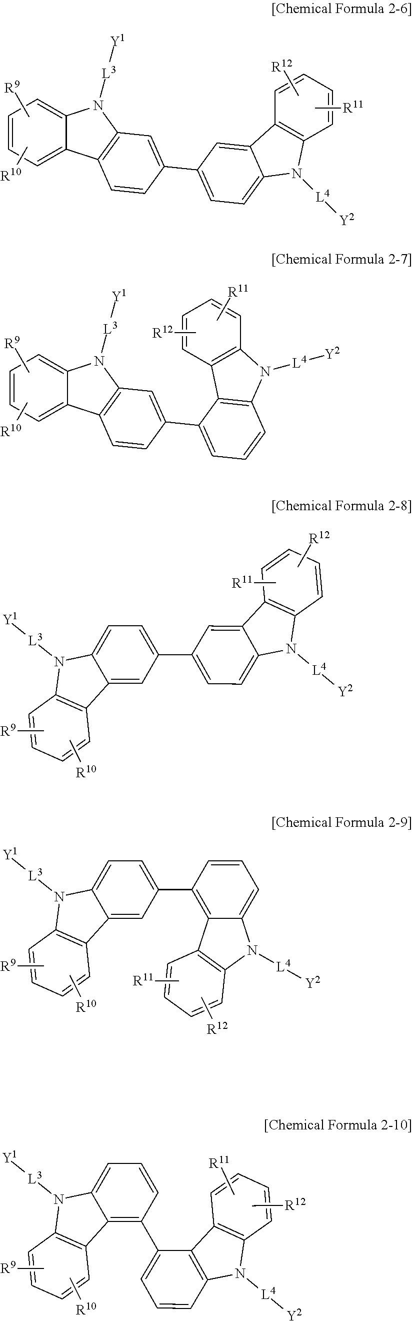

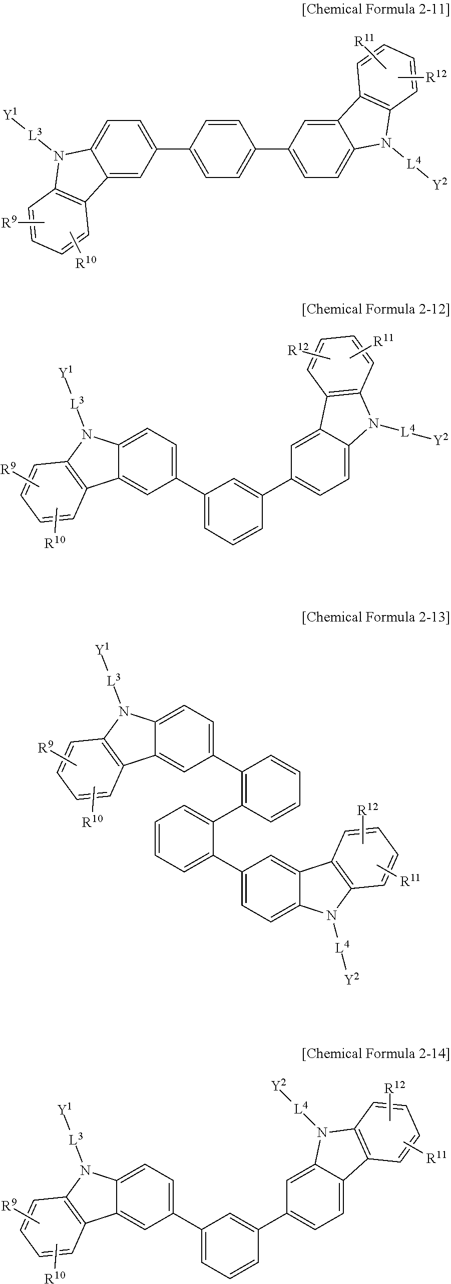

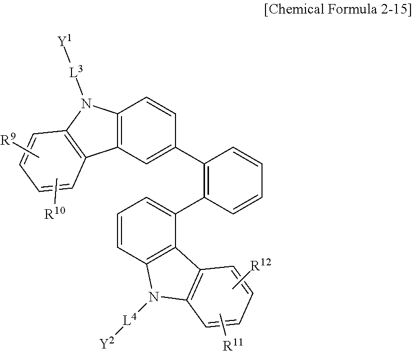

In an implementation, in Chemical Formula 2-1 to Chemical Formula 2-15, R⁹ to R¹² may independently be, e.g., hydrogen, or a substituted or unsubstituted C6 to C12 aryl group. In an implementation, in Chemical Formula 2-1 to Chemical Formula 2-15, the moieties *-L³-Y and *-L⁴-Y² may independently be a moiety of Group I.

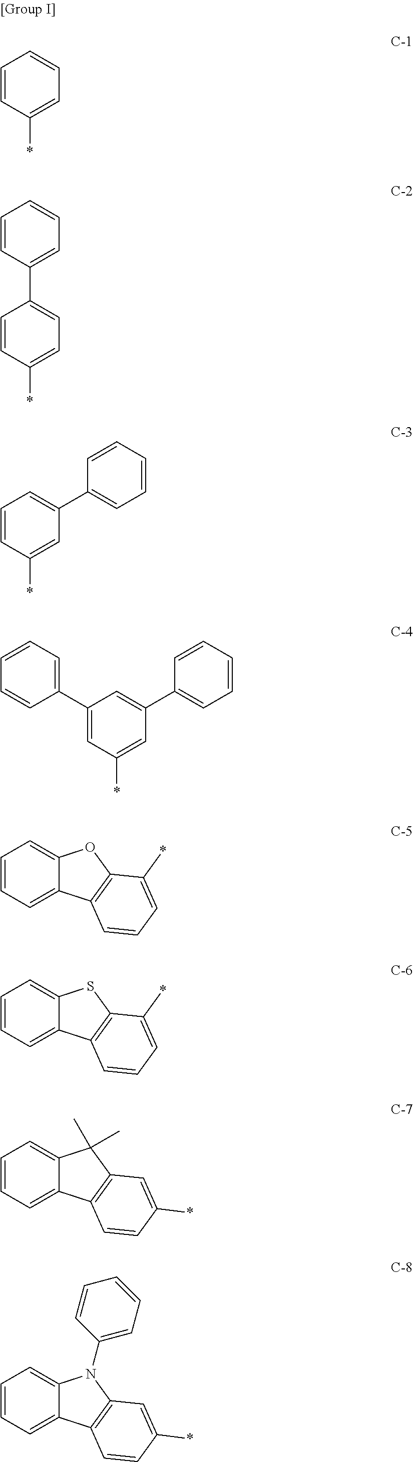

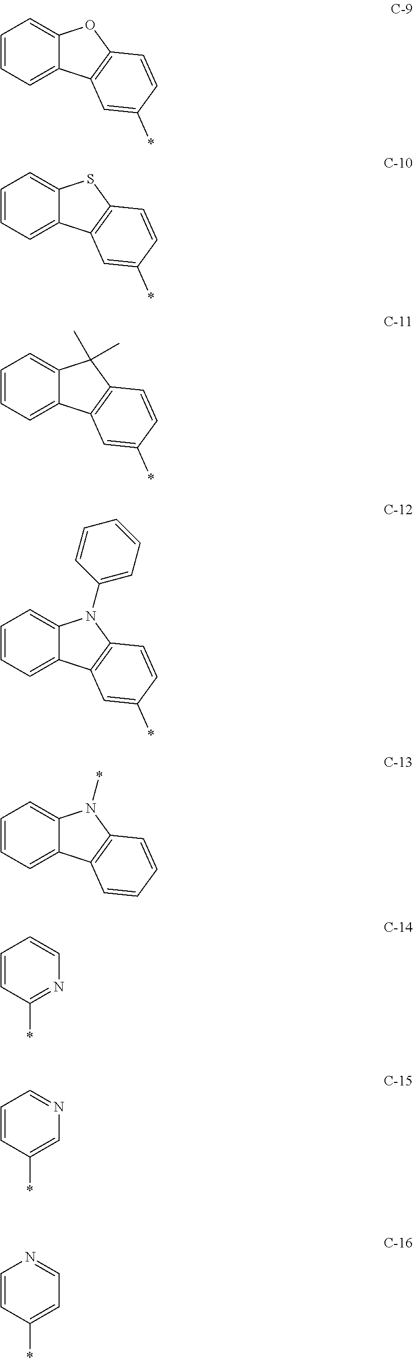

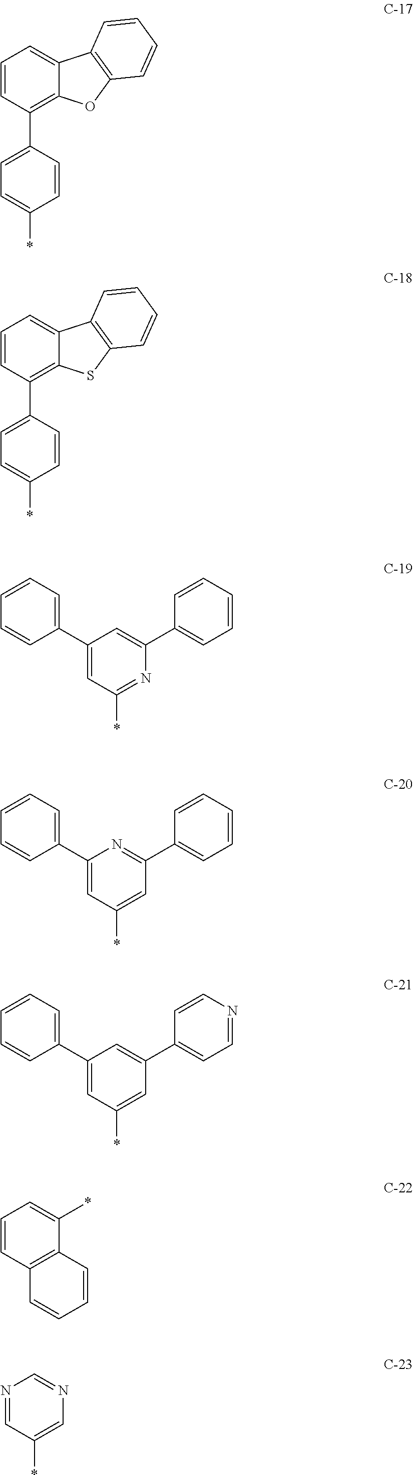

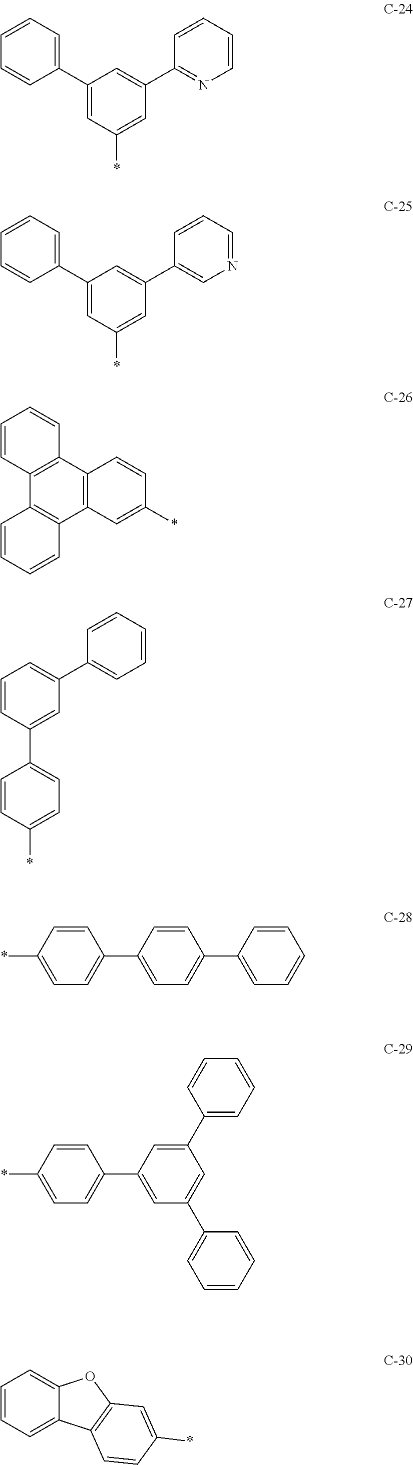

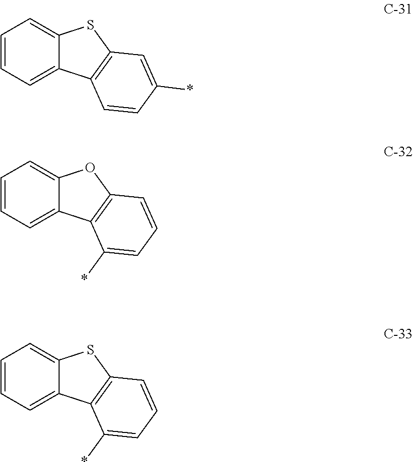 [SMILES: *c1ccc2c(c1)sc1ccccc12 ; *c1cccc2oc3ccccc3c12 ; *c1cccc2sc3ccccc3c12]

In Group I, * is a linking point.

In an implementation, the compound represented by Chemical Formula 2 may be represented by Chemical Formula 2-8.

In an implementation, moieties *-L³-Y¹ and *-L⁴-Y² of Chemical Formula 2-8 may independently be selected from Group I, e.g., one of C-1, C-2, and C-3.

In an implementation, both moieties *-L³-Y¹ and *-L⁴-Y² may be represented by C-2 of Group I.

In an implementation, the second compound represented by a combination of Chemical Formula 3 and Chemical Formula 4 may be represented by Chemical Formula 3A, Chemical Formula 3B, Chemical Formula 3C, Chemical Formula 3D, or Chemical Formula 3E.

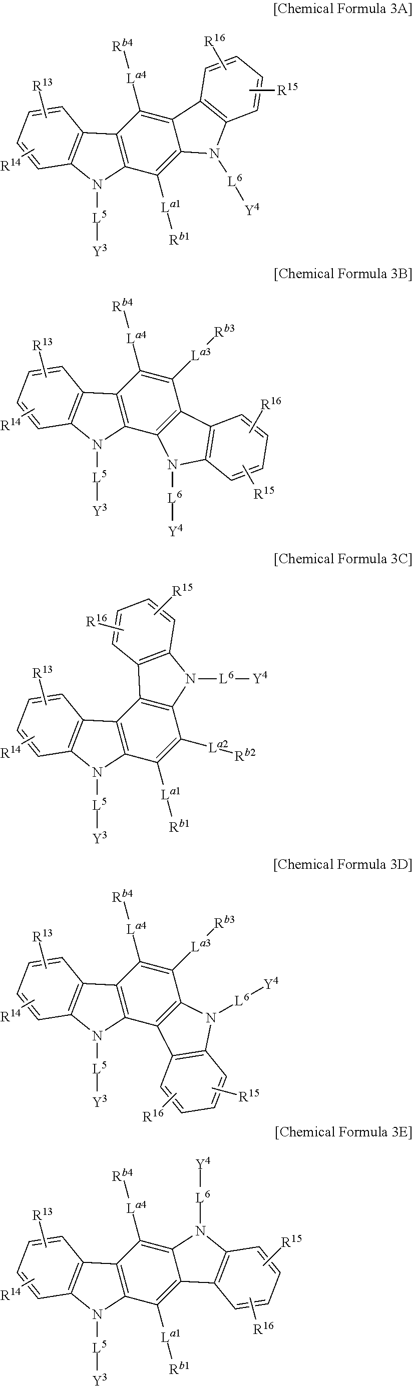

In Chemical Formula 3A to Chemical Formula 3E, Y³ and Y⁴, L⁵ and L⁶, and R¹³ to R¹⁶ may be the same as described above.

L^{a1} to L^{a4} may be defined the same as L⁵ and L⁶, and R^{b1} to R⁴ may be defined the same as R¹³ to R¹⁶.

In an implementation, Y³ and Y⁴ of Chemical Formulae 3 and 4 may independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{b1} to R⁴ and R¹³ to R¹⁶ may independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, Y³ and Y⁴ of Chemical Formulae 3 and 4 may independently be a group of the following Group II.

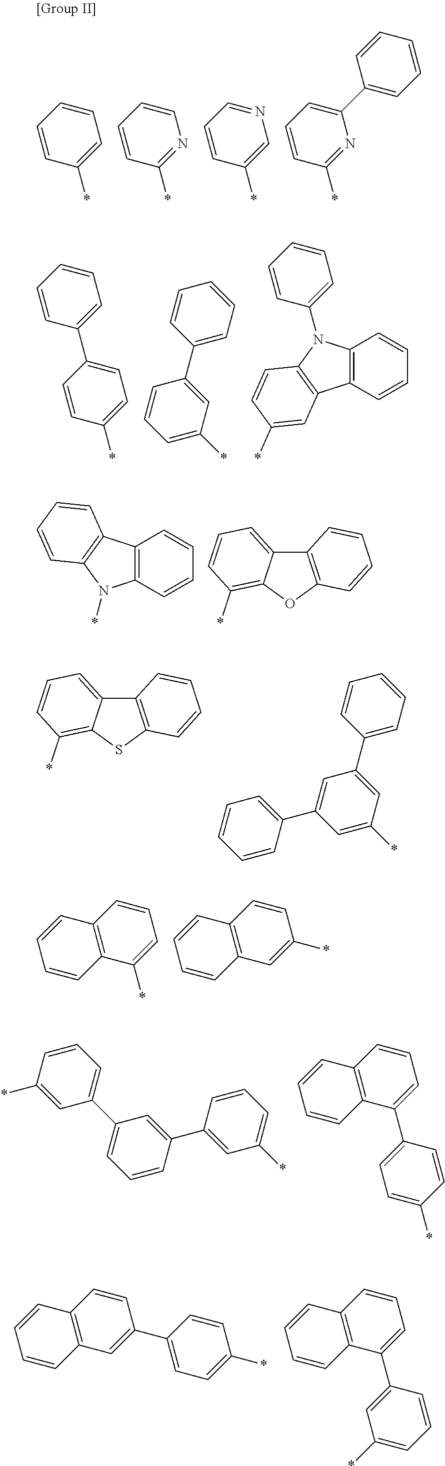

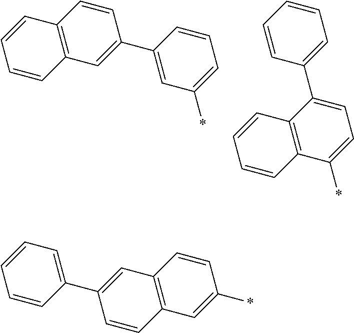

In Group II, * is a linking point to L⁵ and L⁶.

In an implementation, R^{b1} to R⁴ and R¹³ to R¹⁶ may independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{b1} to R⁴ and R¹³ to R¹⁶ may independently be, e.g., hydrogen, deuterium, a cyano group, or a substituted or unsubstituted phenyl group.

In an implementation, R^{b1} to R⁴ may each be, e.g., hydrogen, and R¹³ to R¹⁶ may independently be, e.g., hydrogen or a substituted or unsubstituted phenyl group.

In an implementation, the second compound may be represented by Chemical Formula 2-8.

In an implementation, Y¹ and Y² of Chemical Formula 2-8 may independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L³ and L⁴ may independently be, e.g., a single bond or a substituted or unsubstituted C6 to C20 arylene group, and R⁹ to R¹² may independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, moieties *-L³-Y¹ and *-L⁴-Y² in Chemical Formula 2-8 may be, e.g., represented by C-2 of Group I.

In an implementation, the second compound may be a compound of the following Group 2.

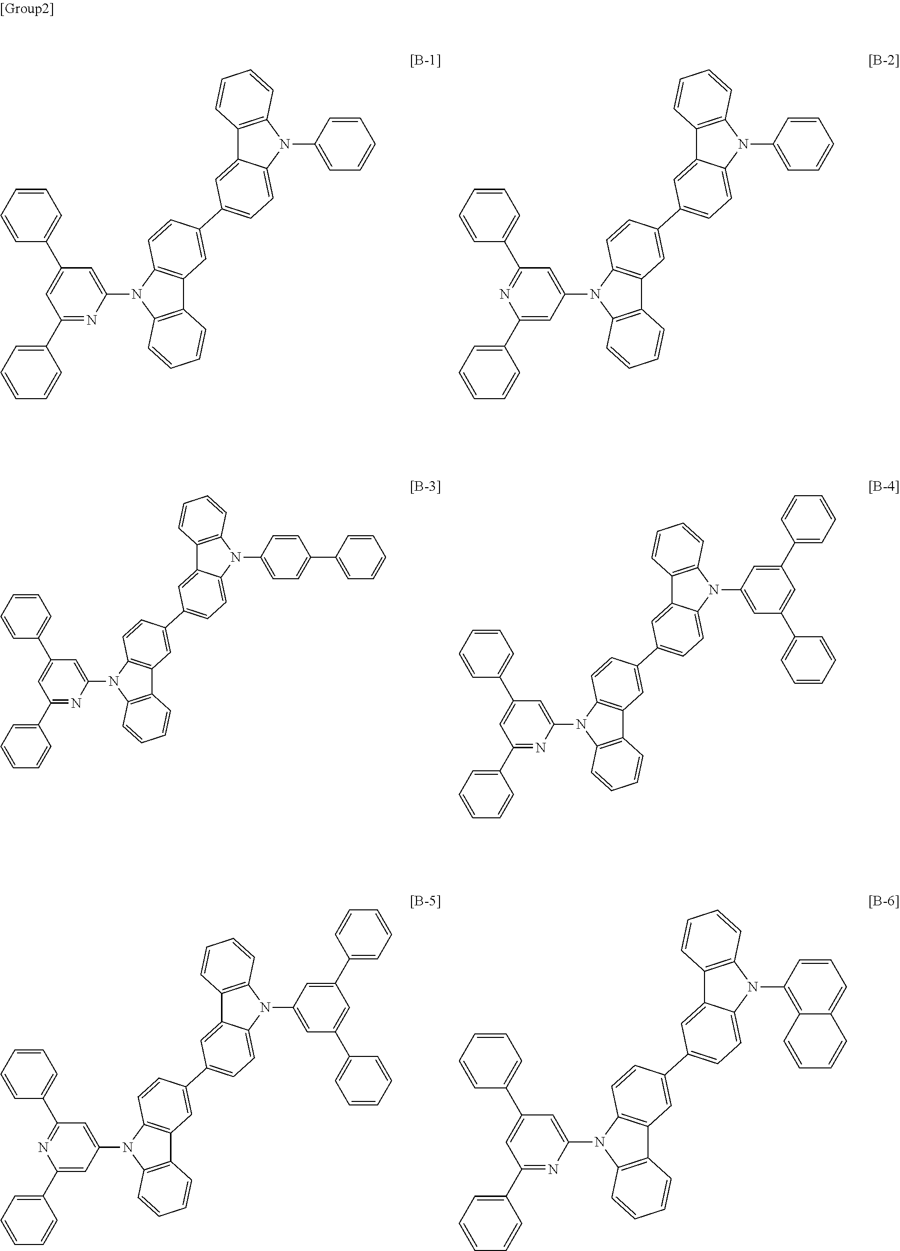

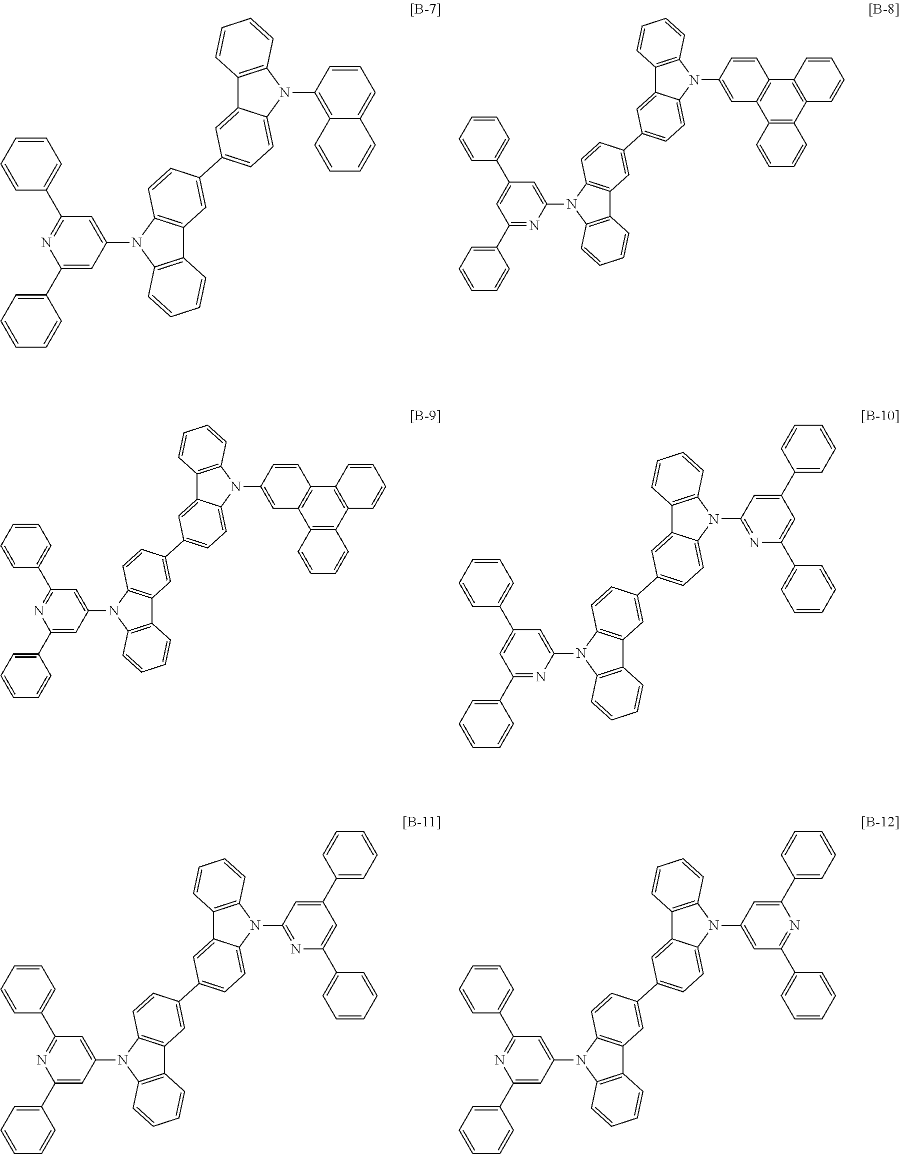

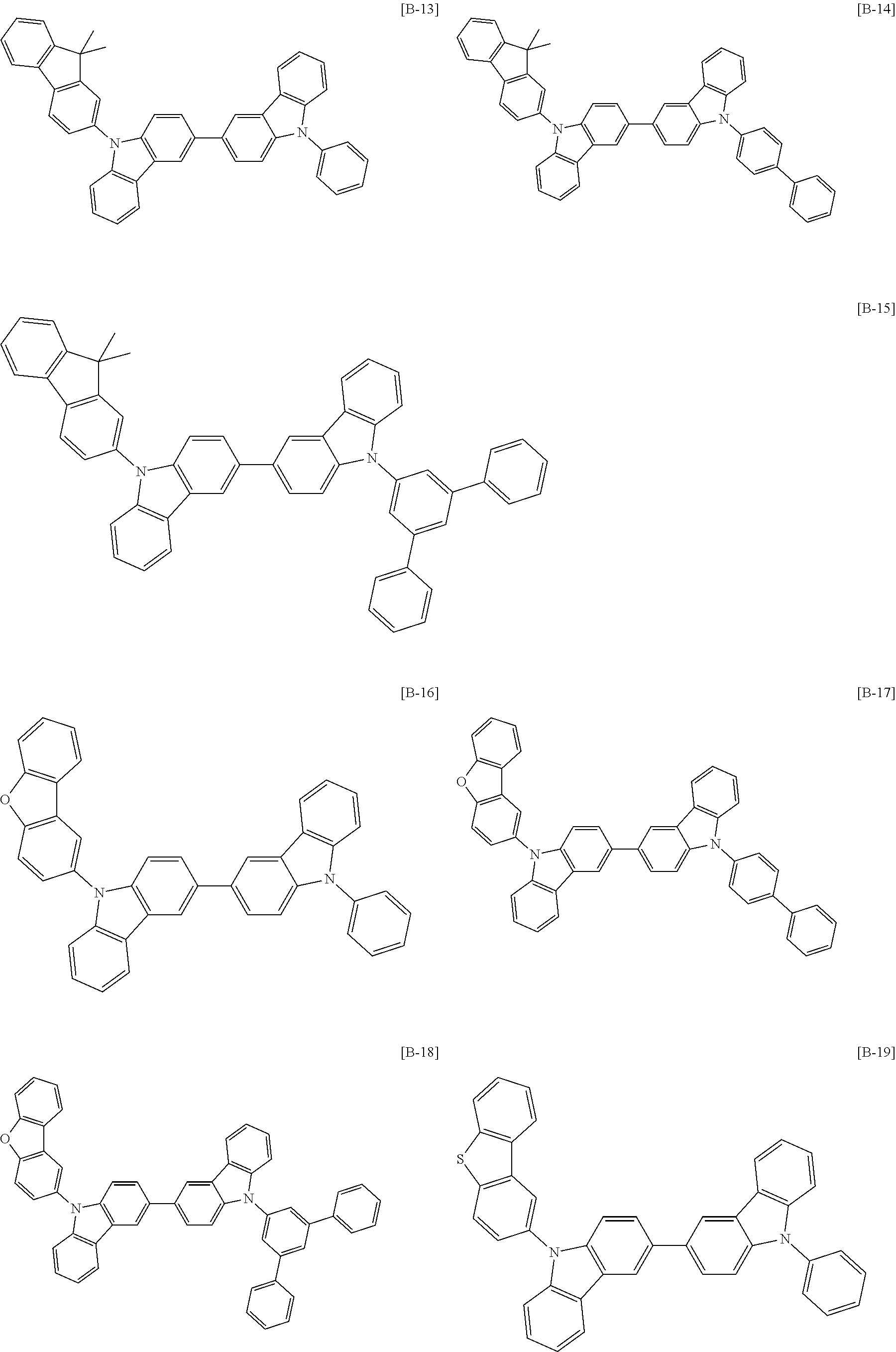

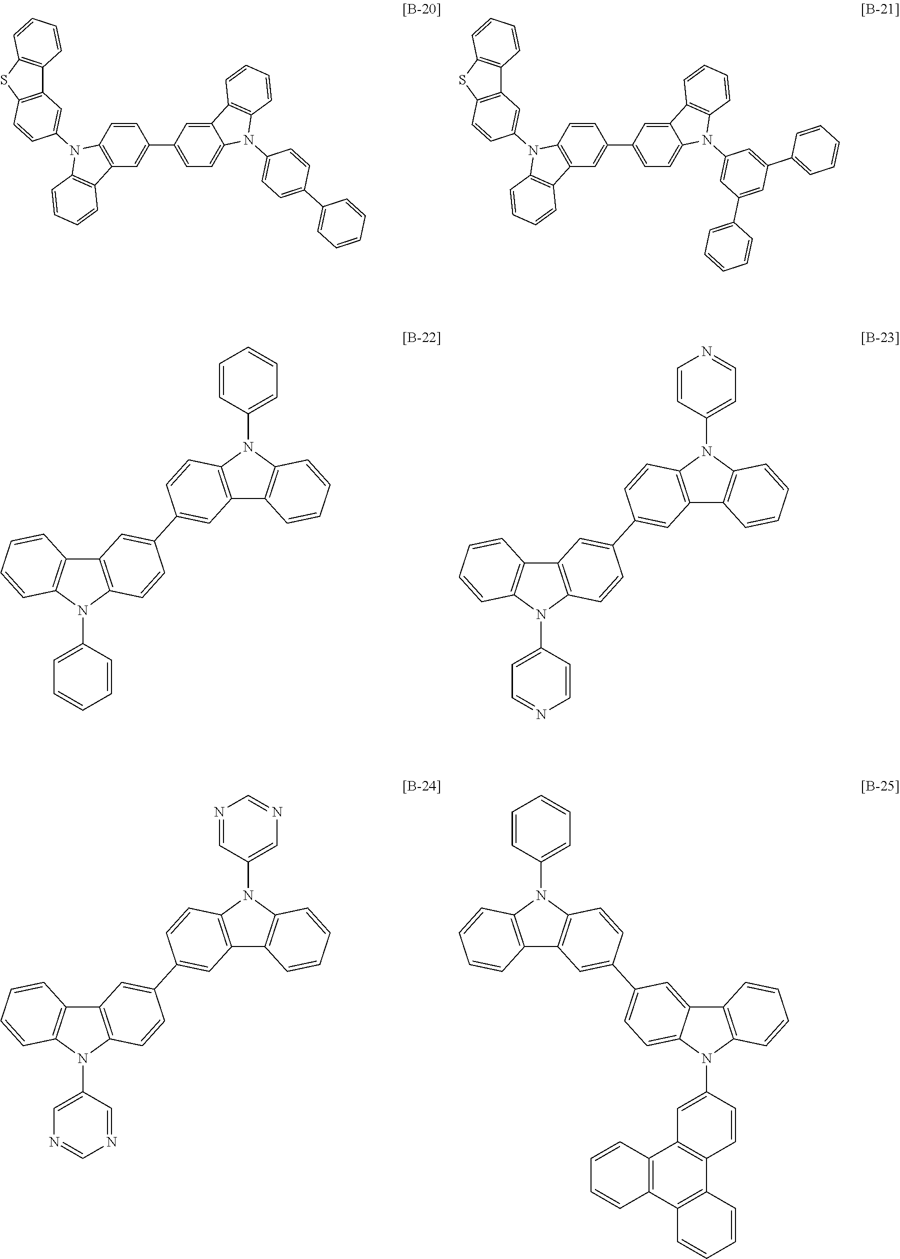 [SMILES: c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1]

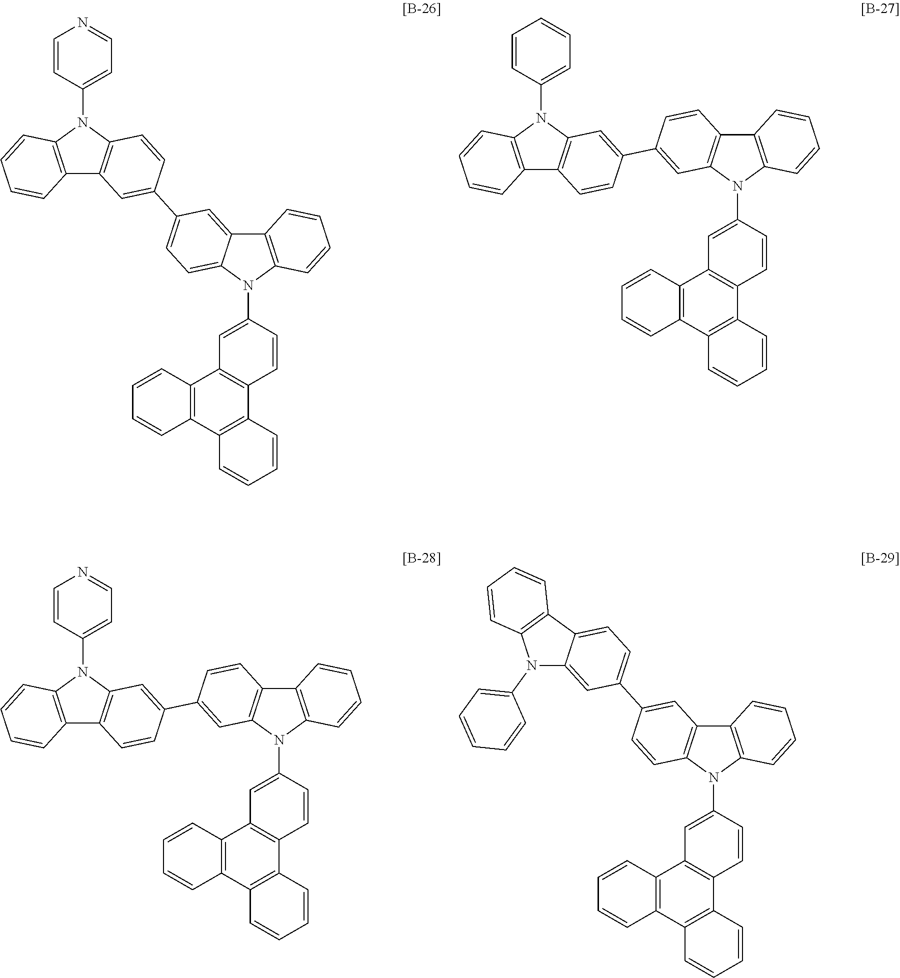 [SMILES: c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1]

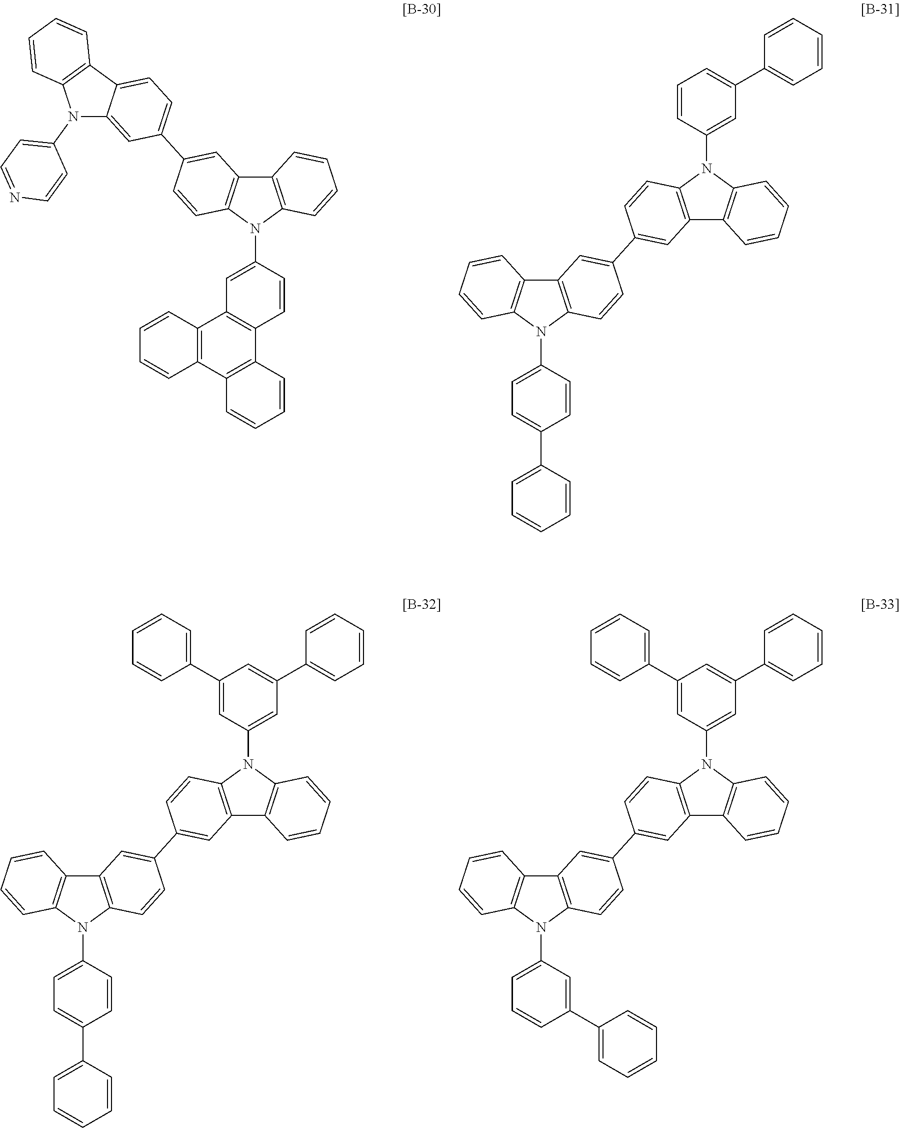 [SMILES: c1ccc2c(c1)c1ccccc1c1cc(-n3c4ccccc4c4cc(-c5ccc6c7ccccc7n(-c7ccncc7)c6c5)ccc43)ccc21 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

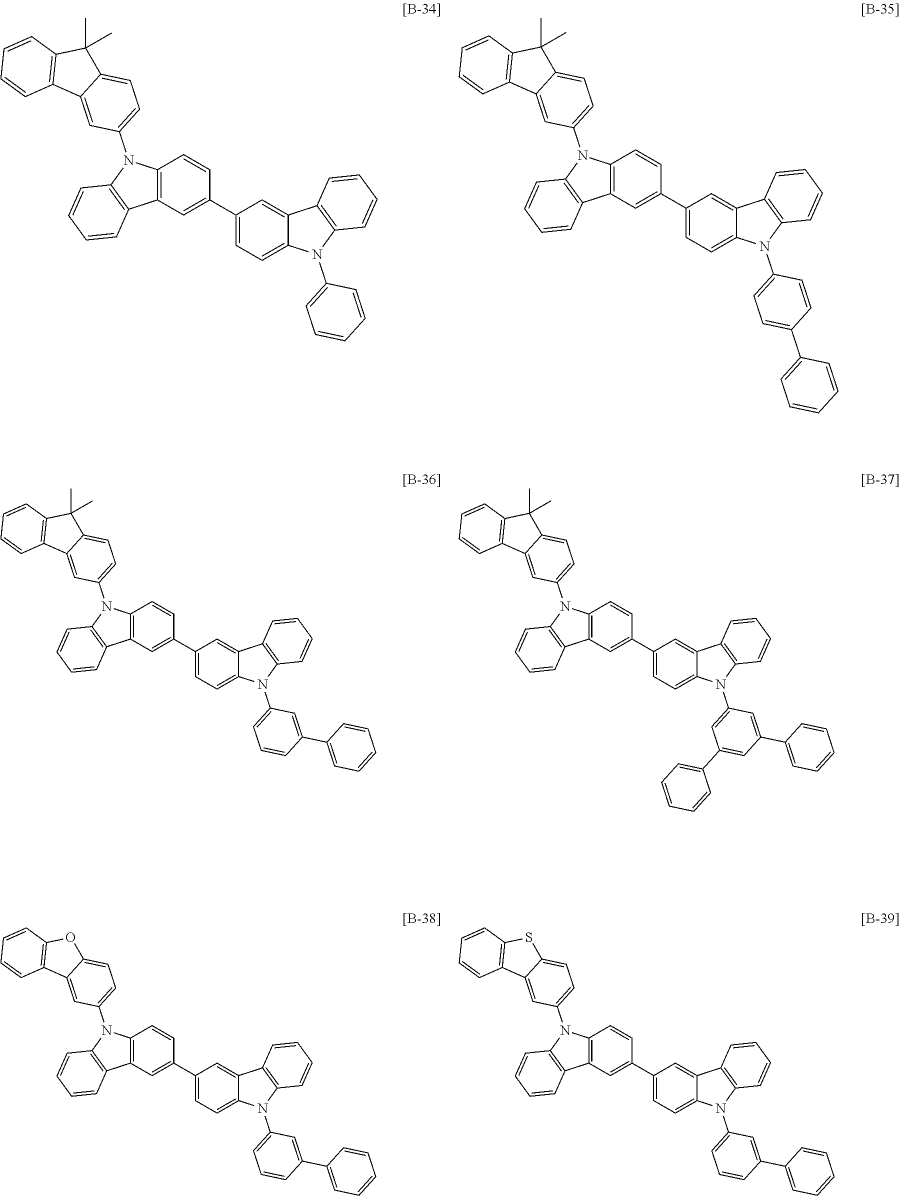

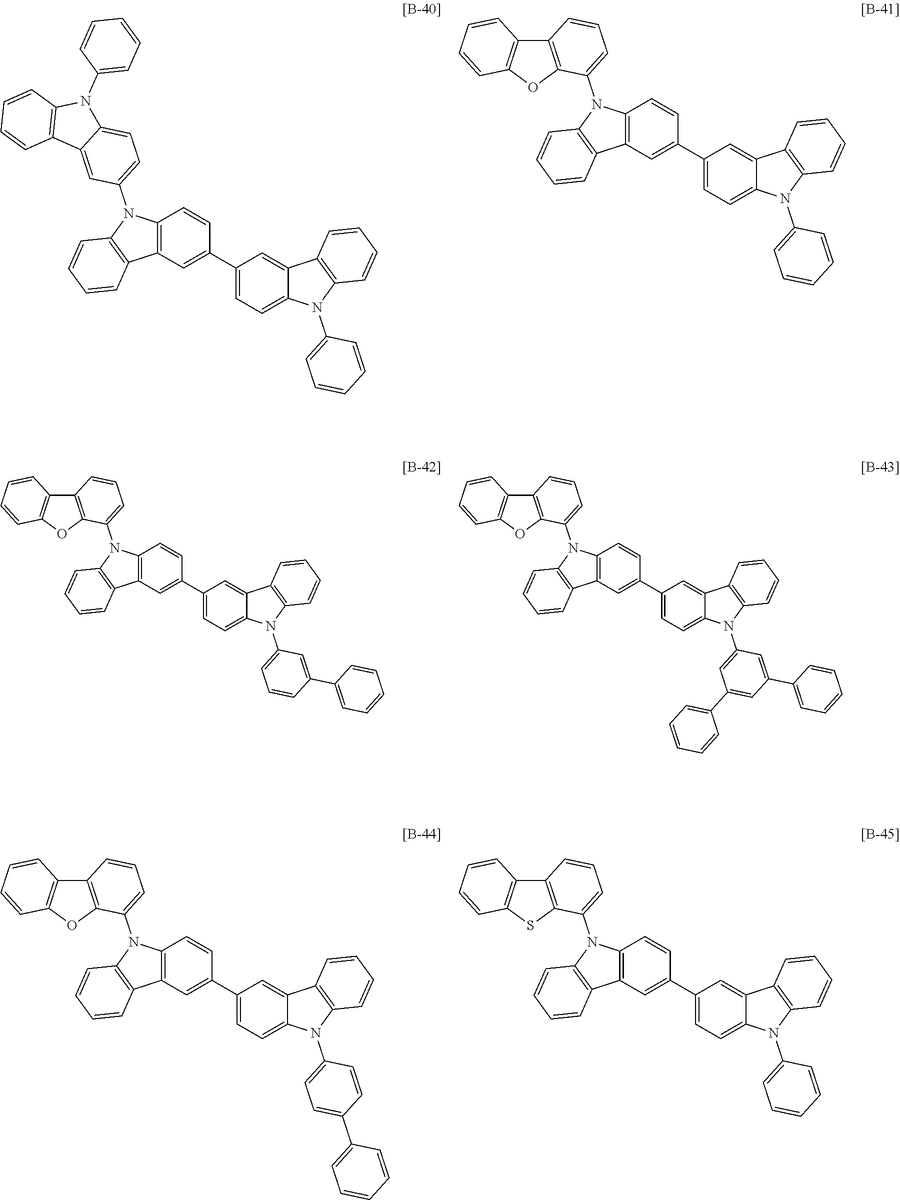

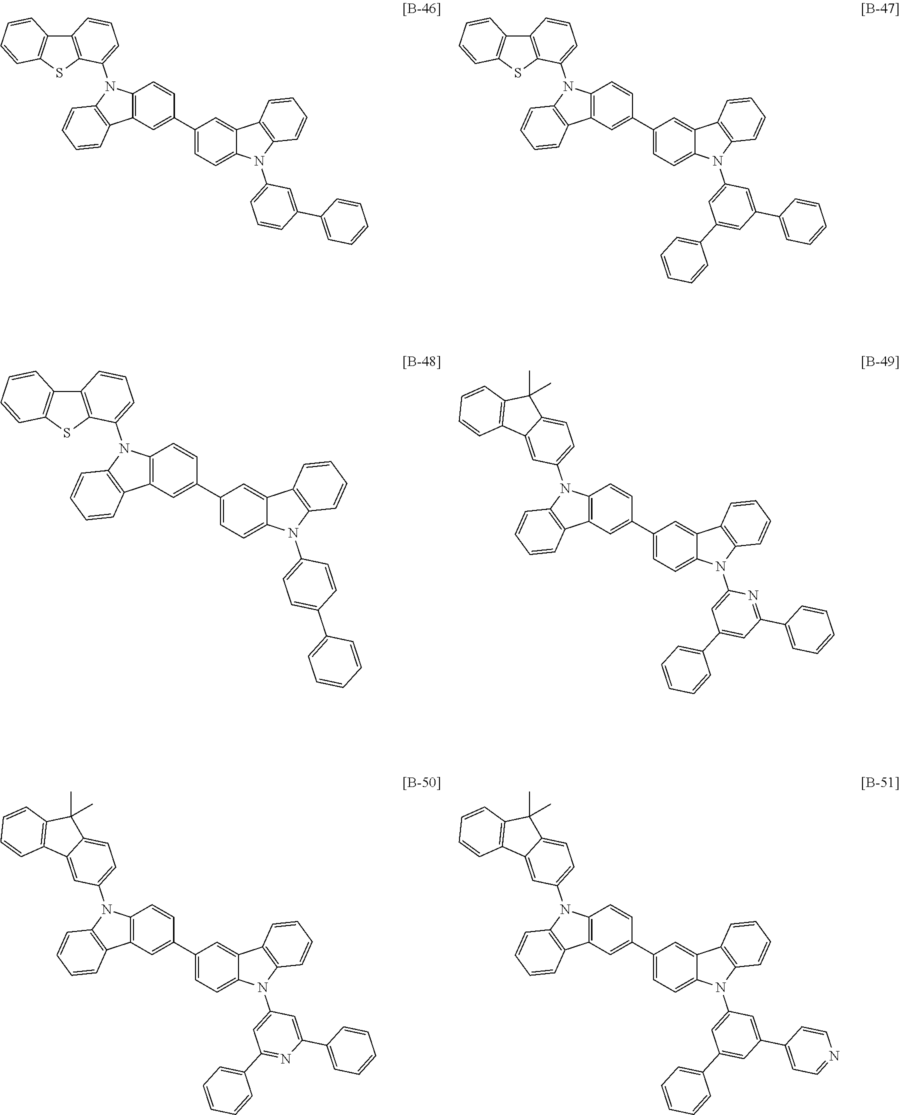

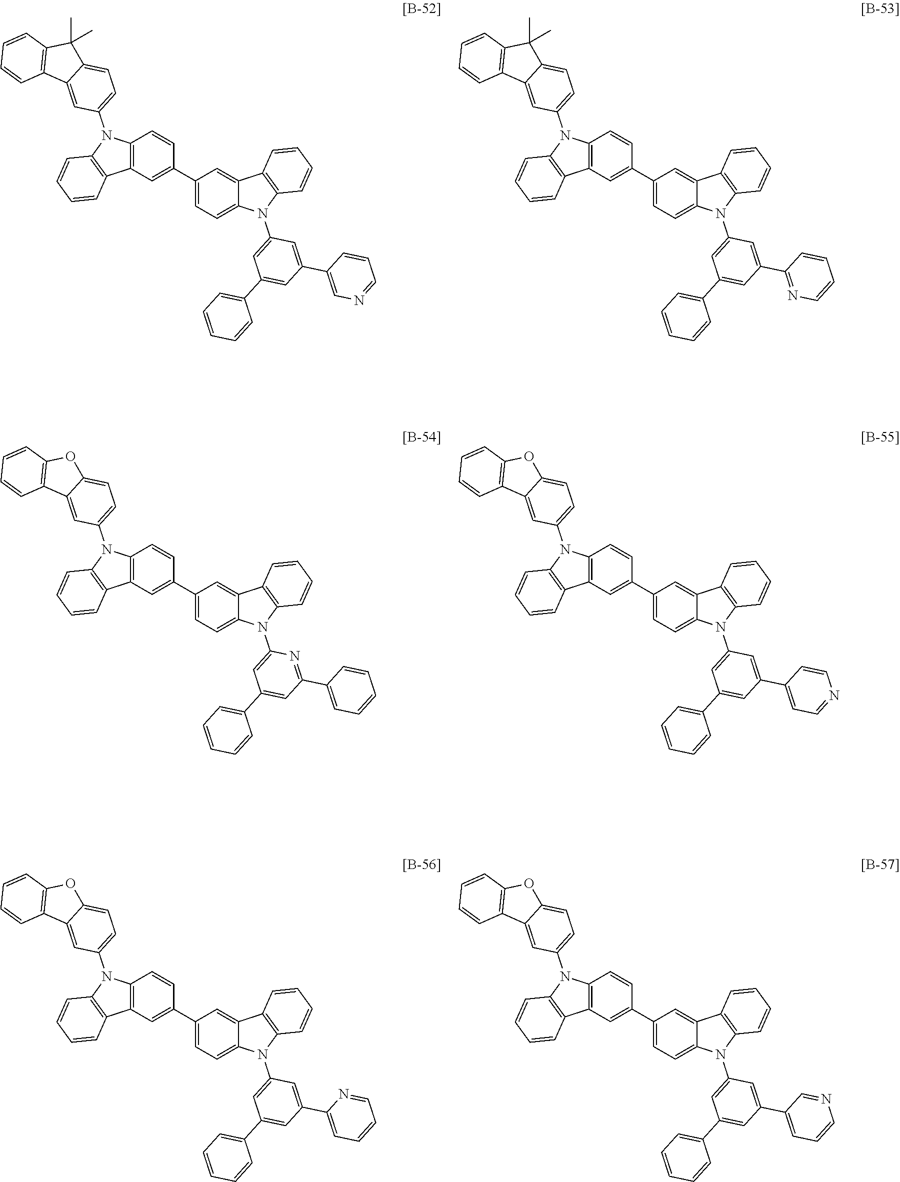

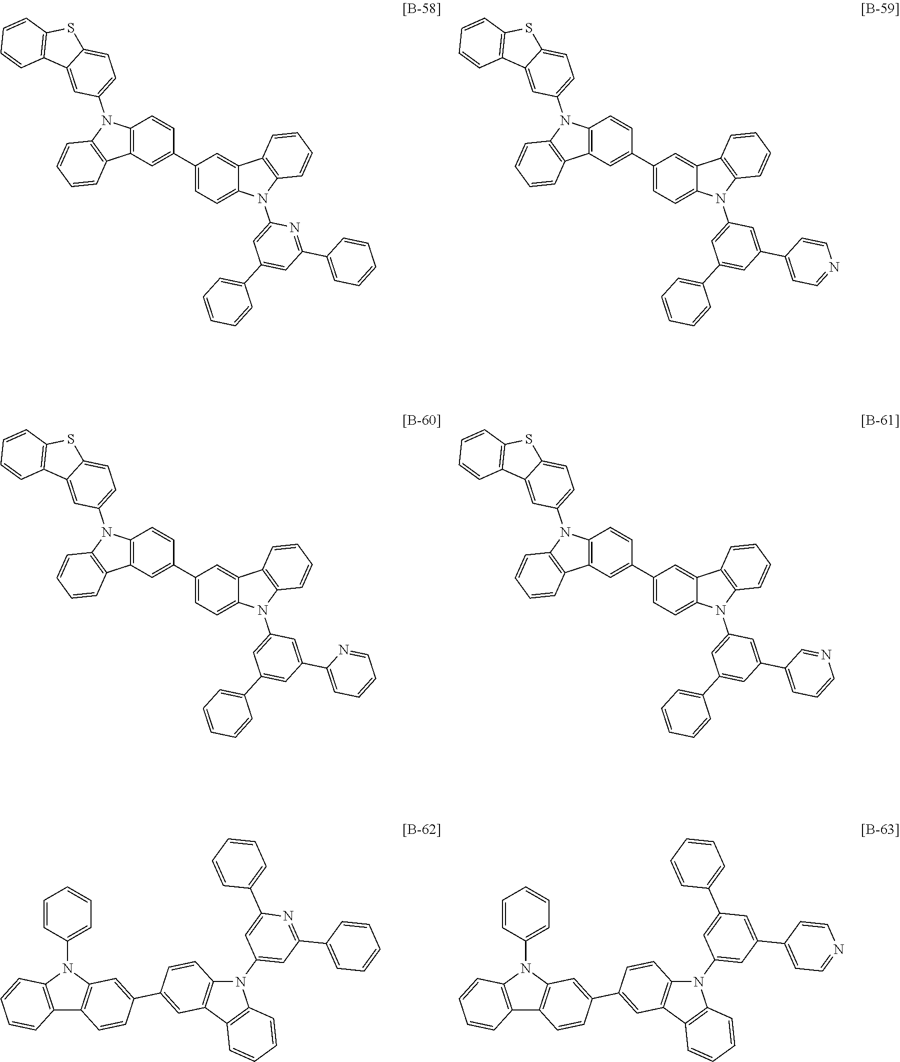

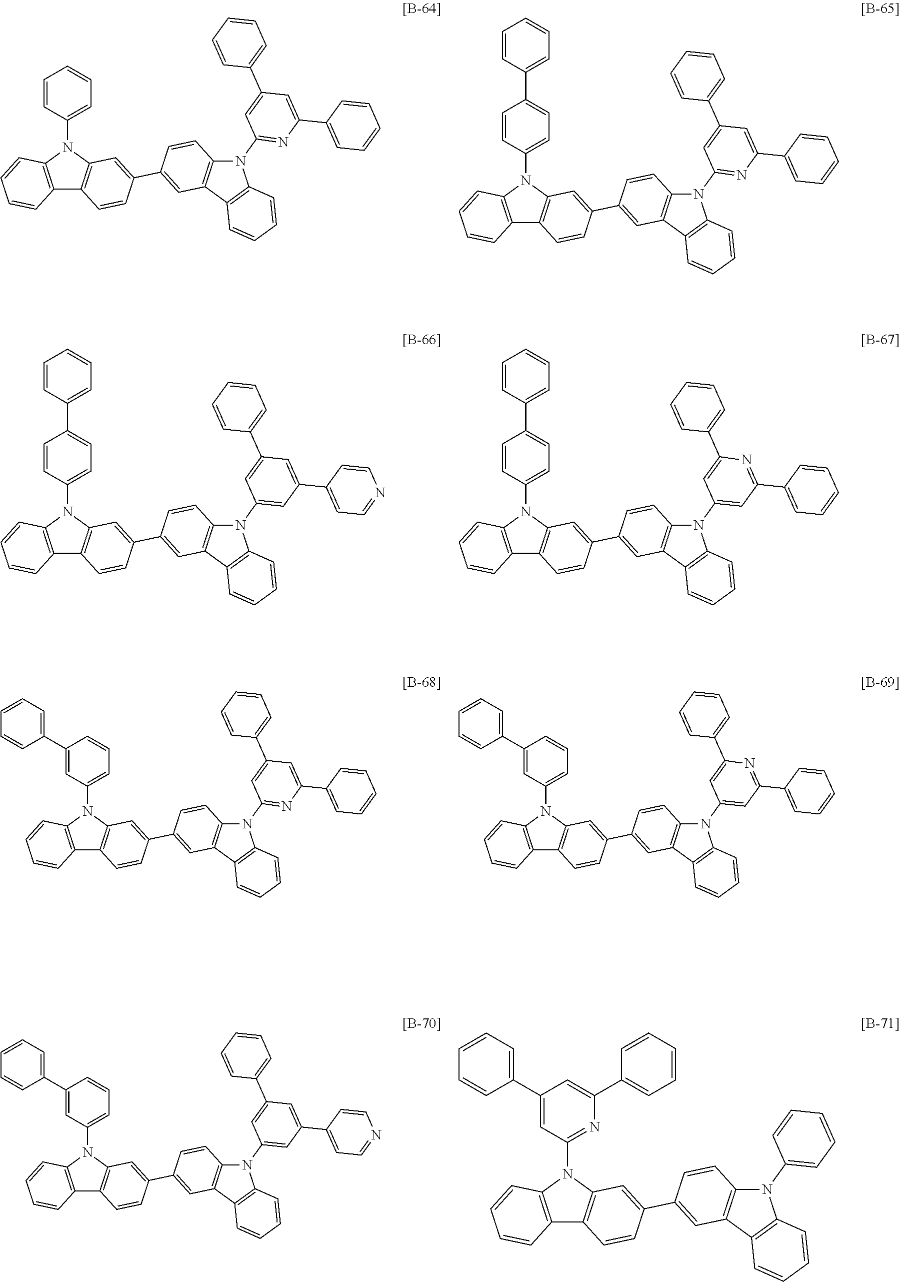

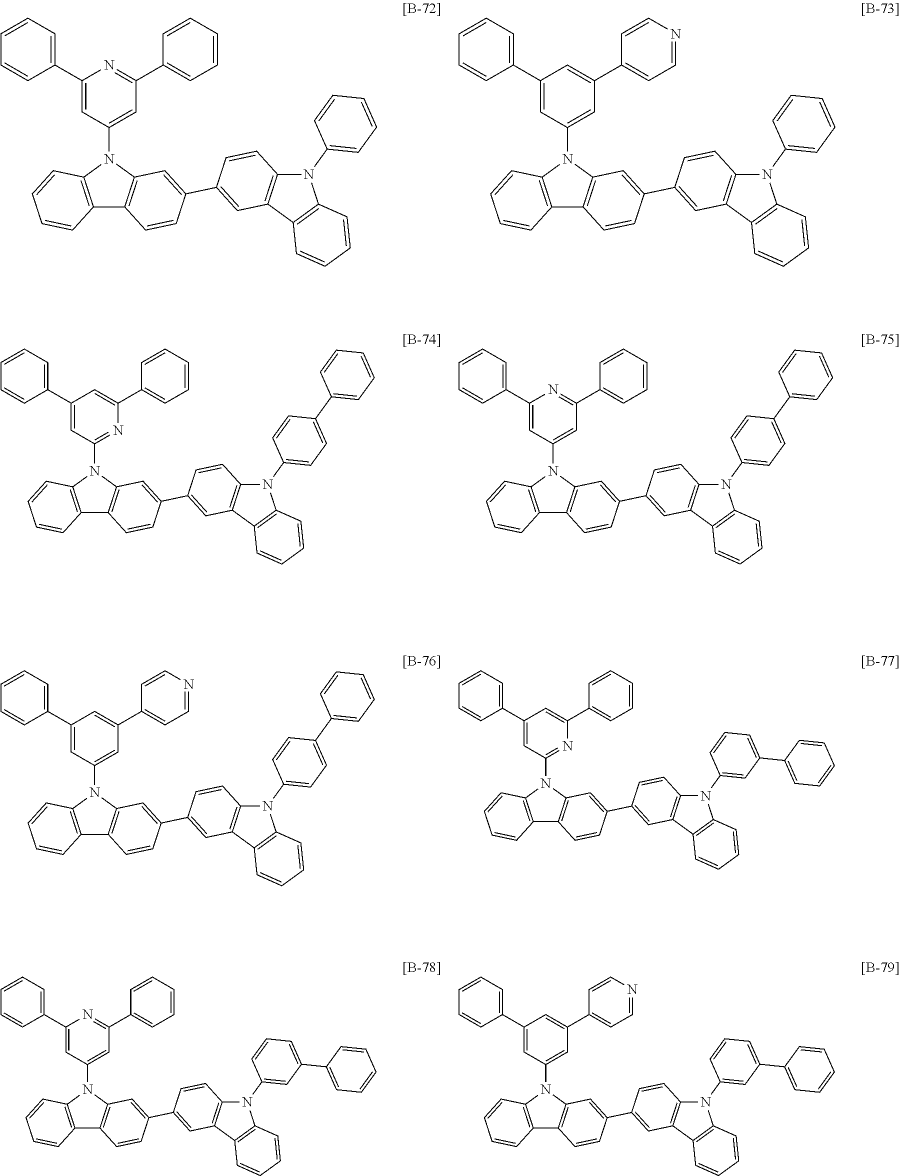

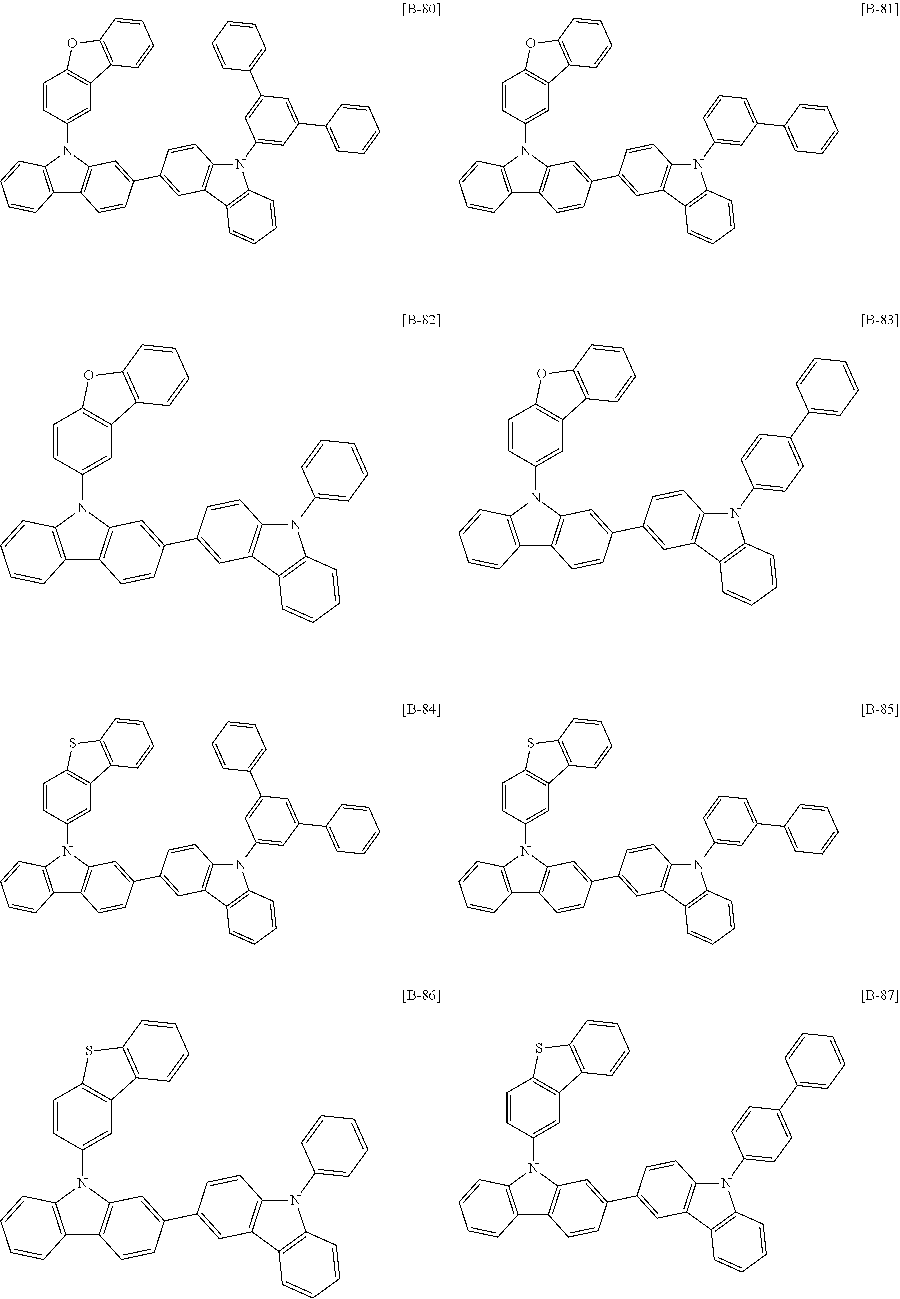

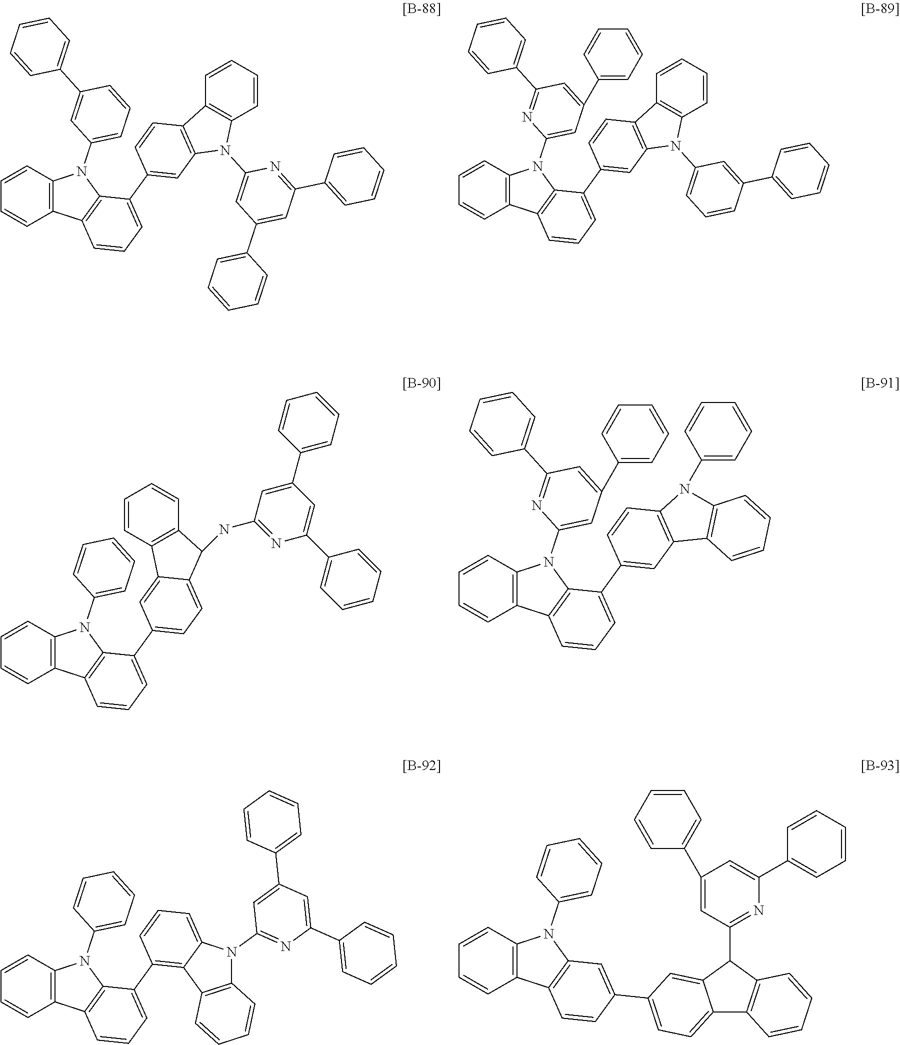

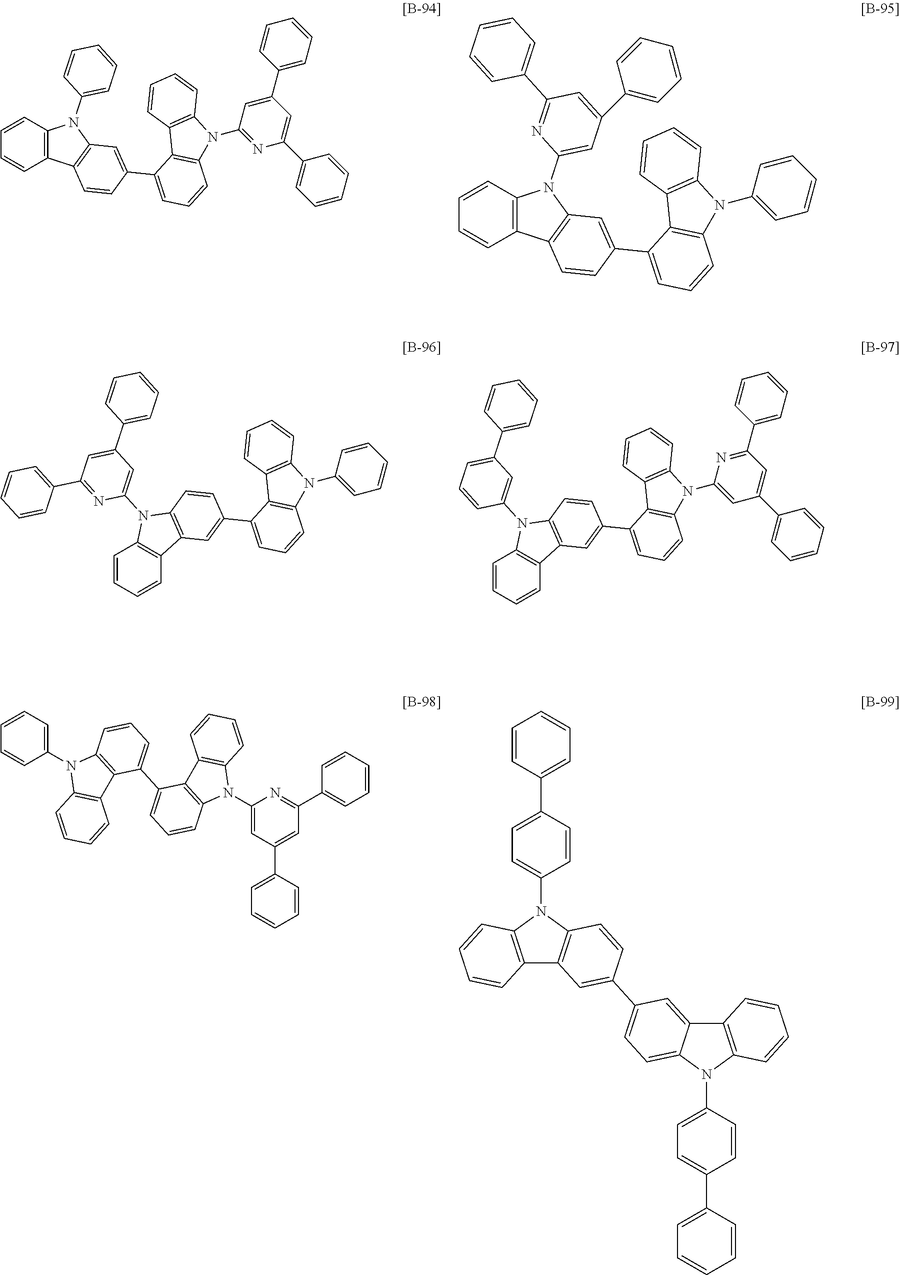

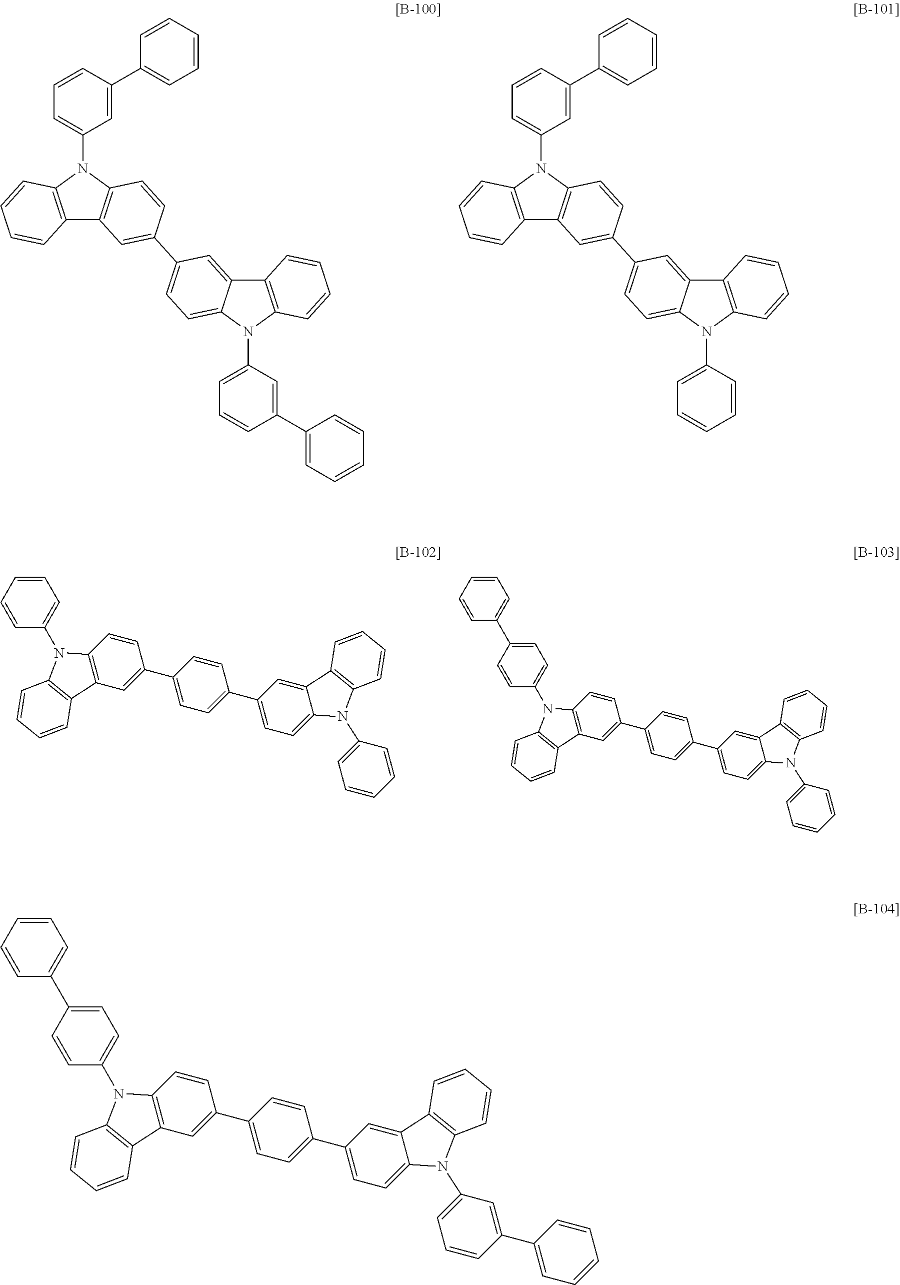 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

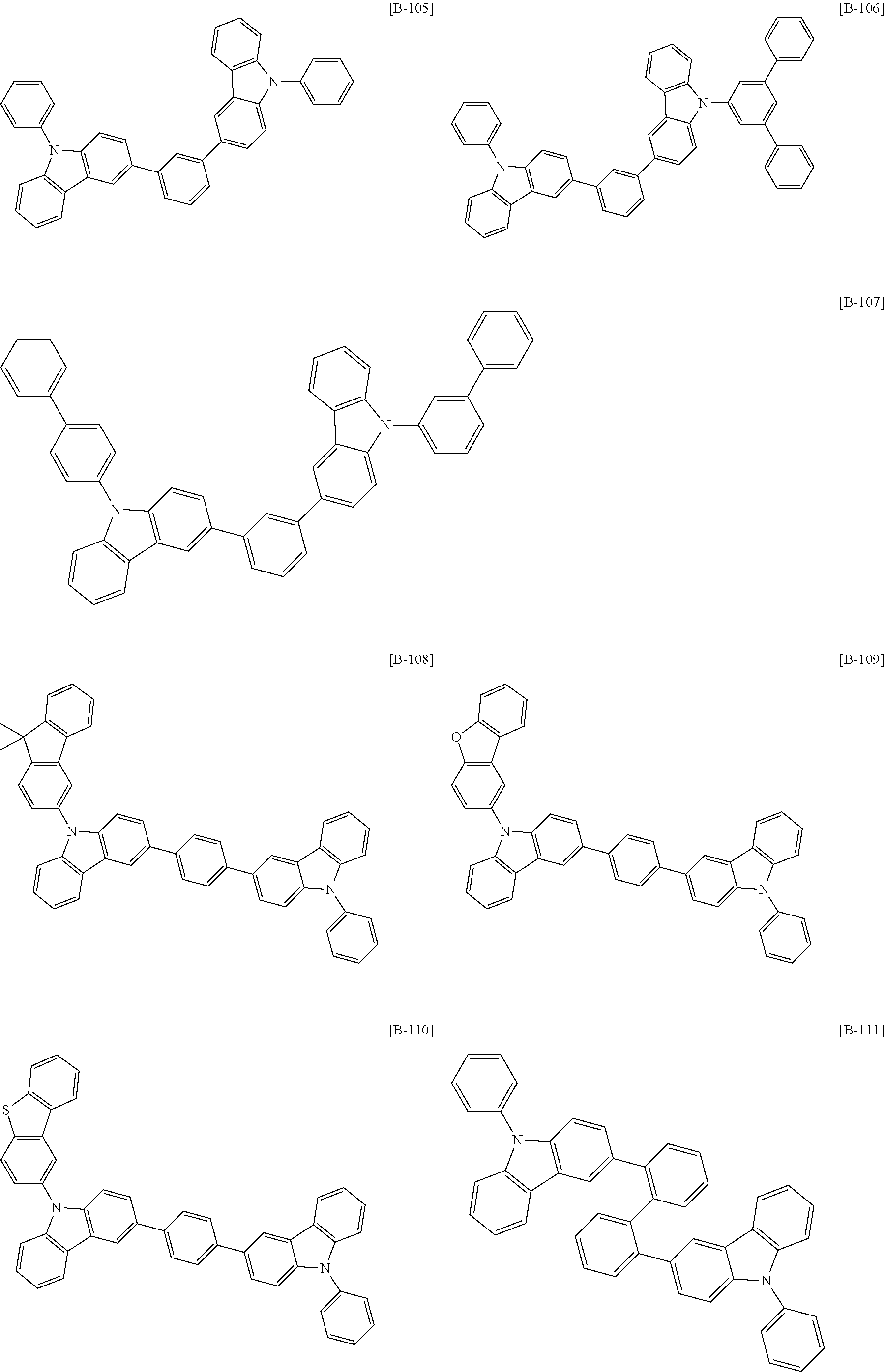

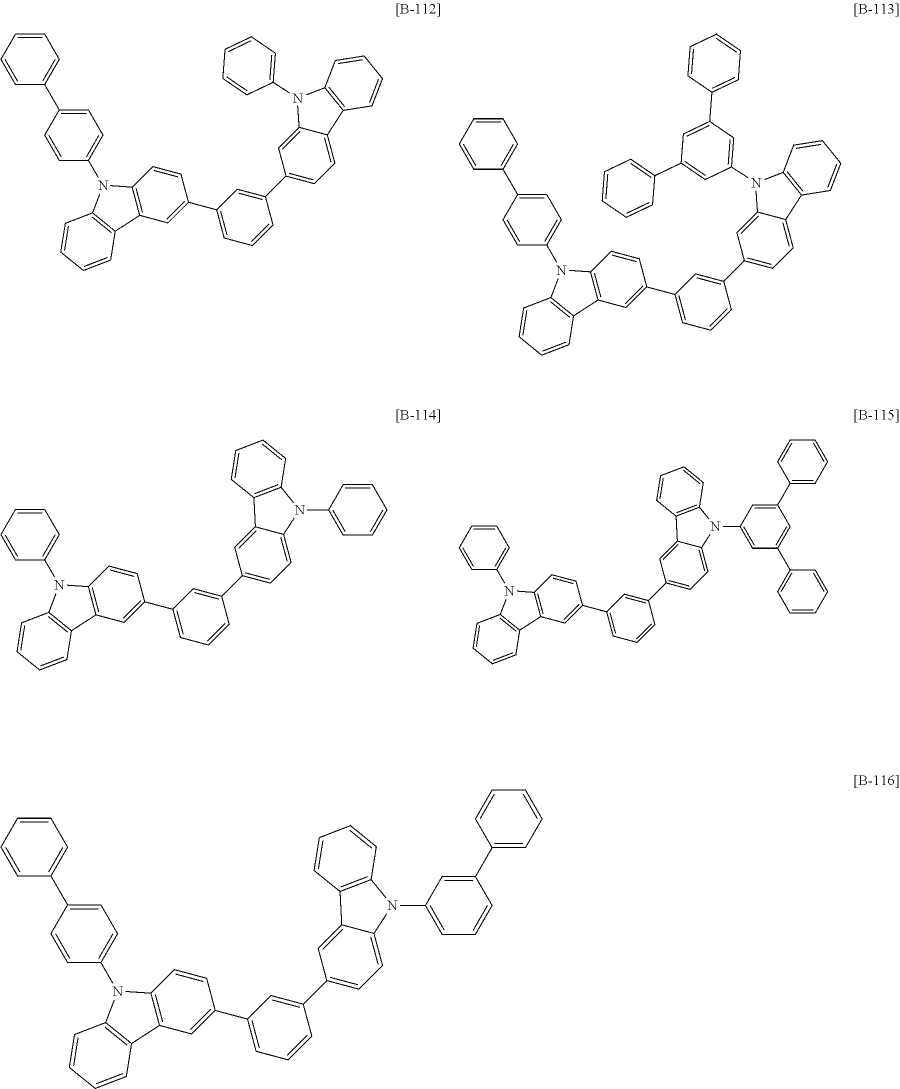 [SMILES: c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1]

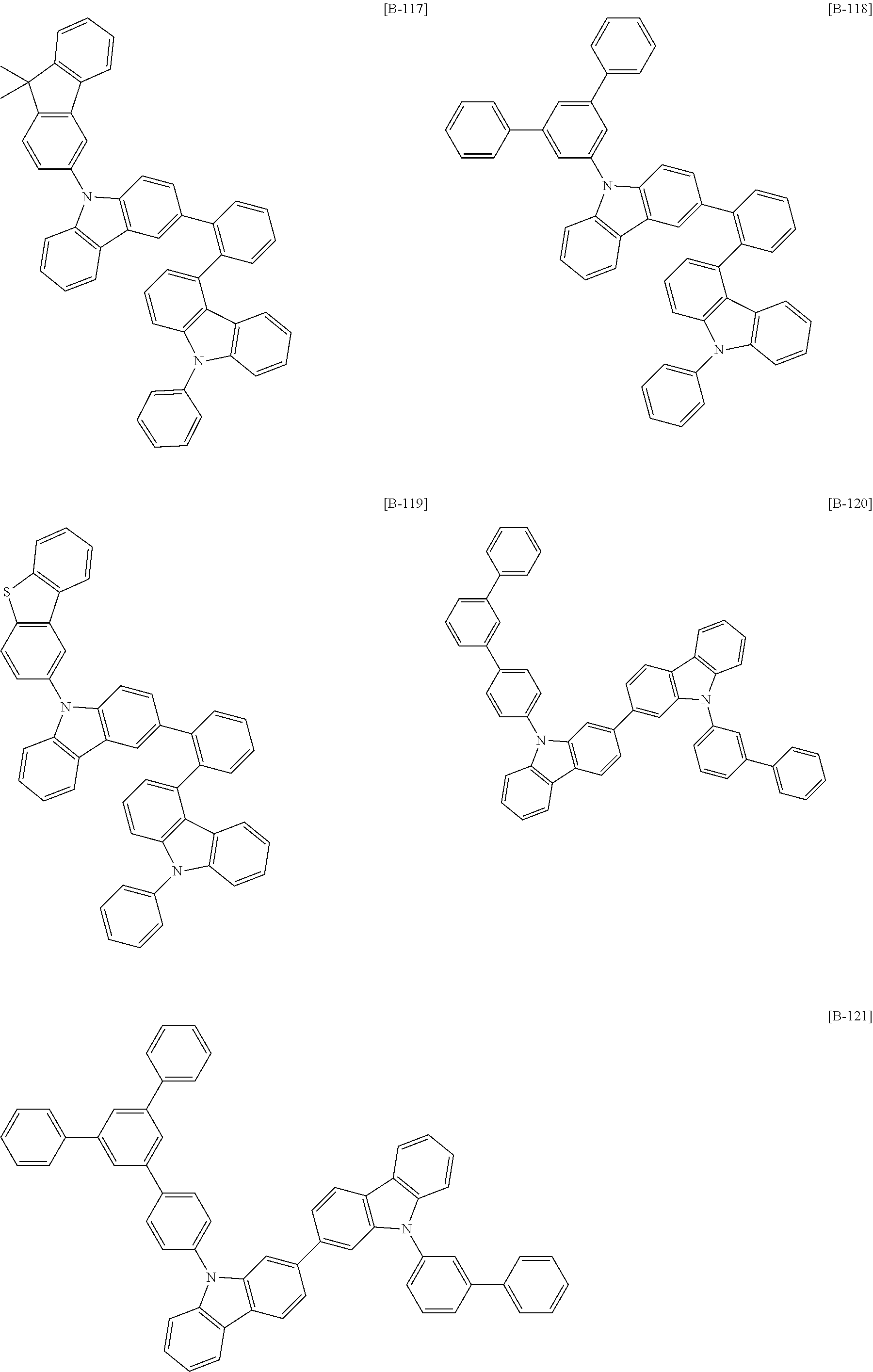 [SMILES: CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1]

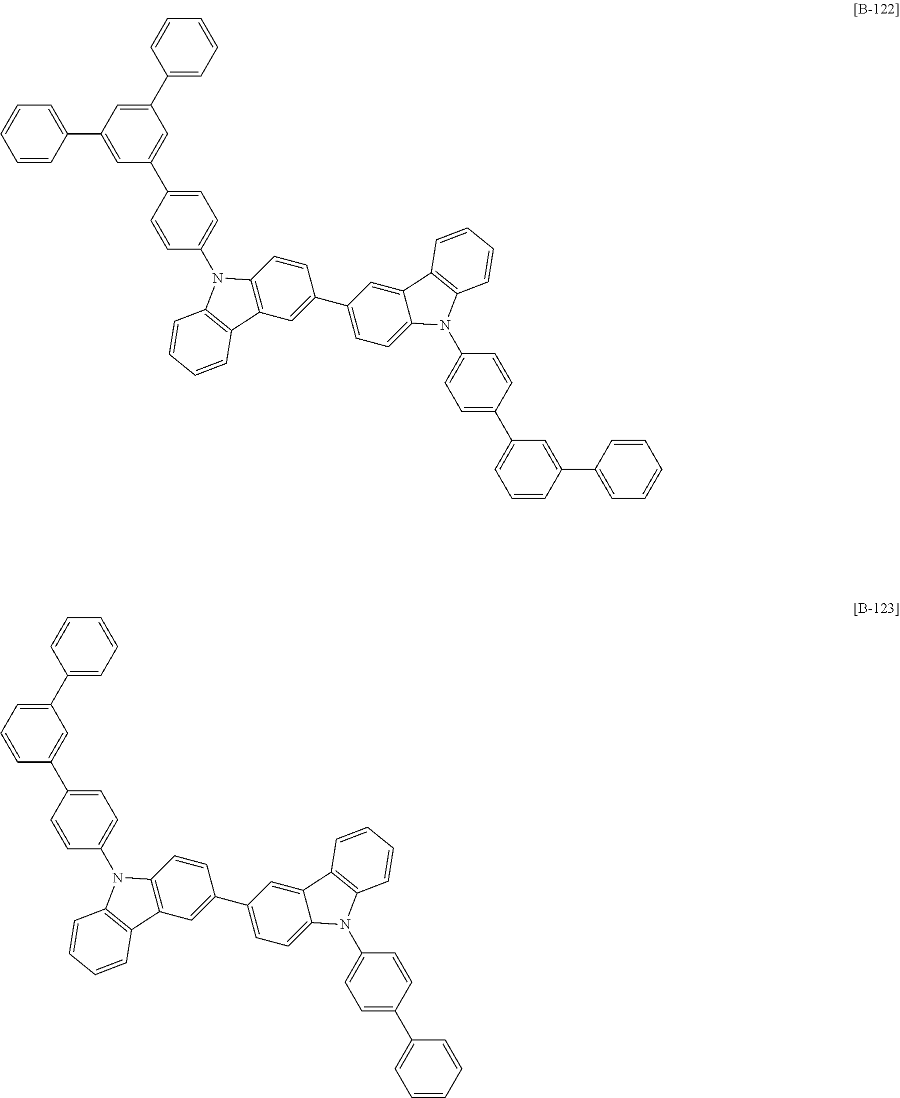 [SMILES: c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

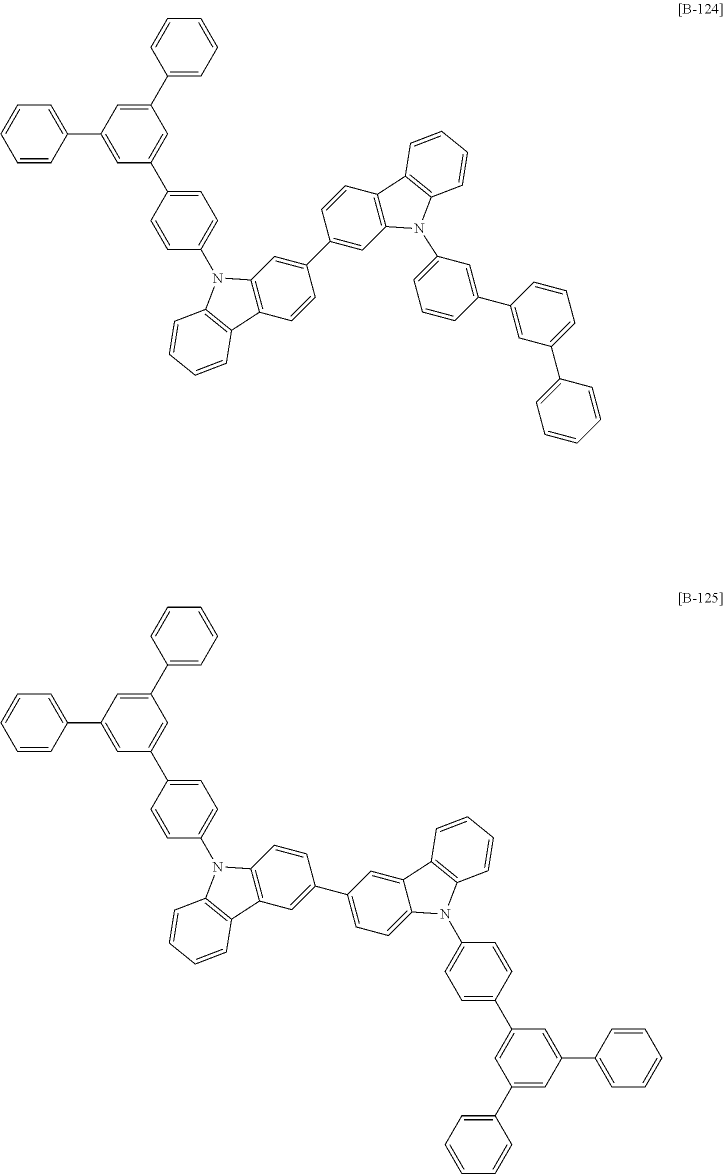 [SMILES: c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

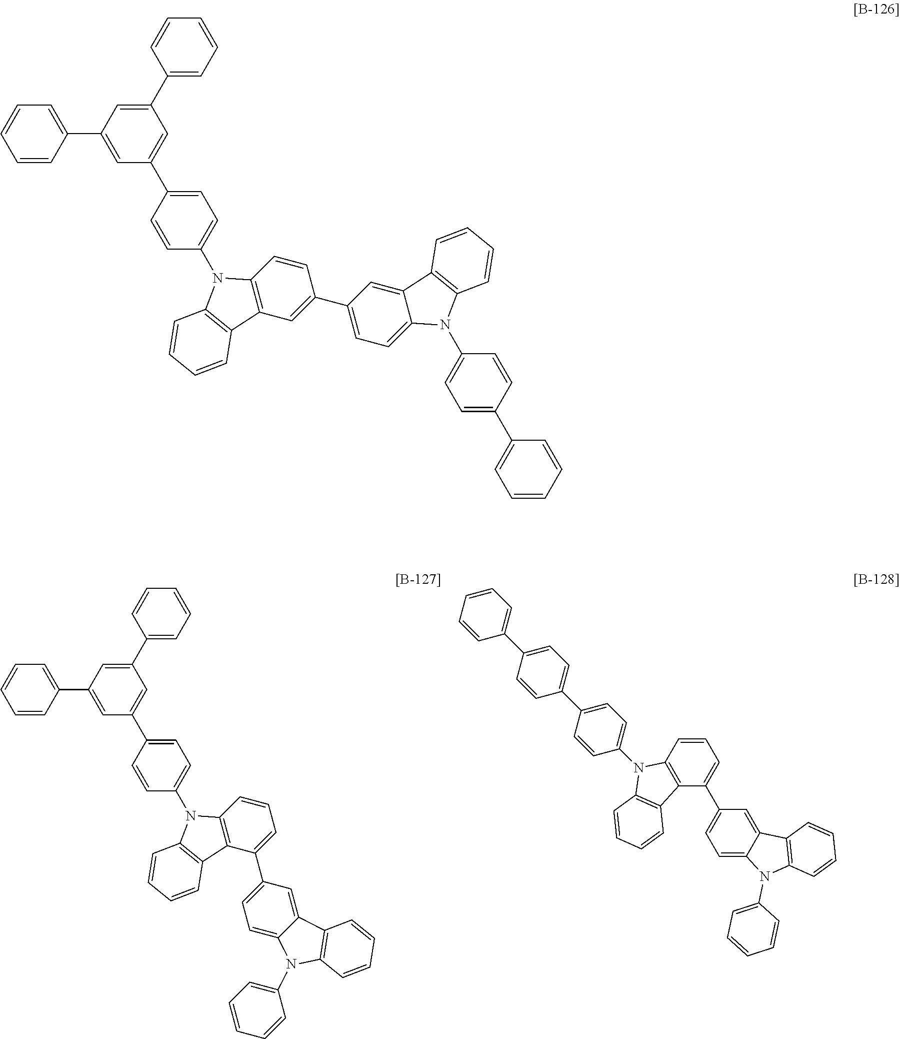 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

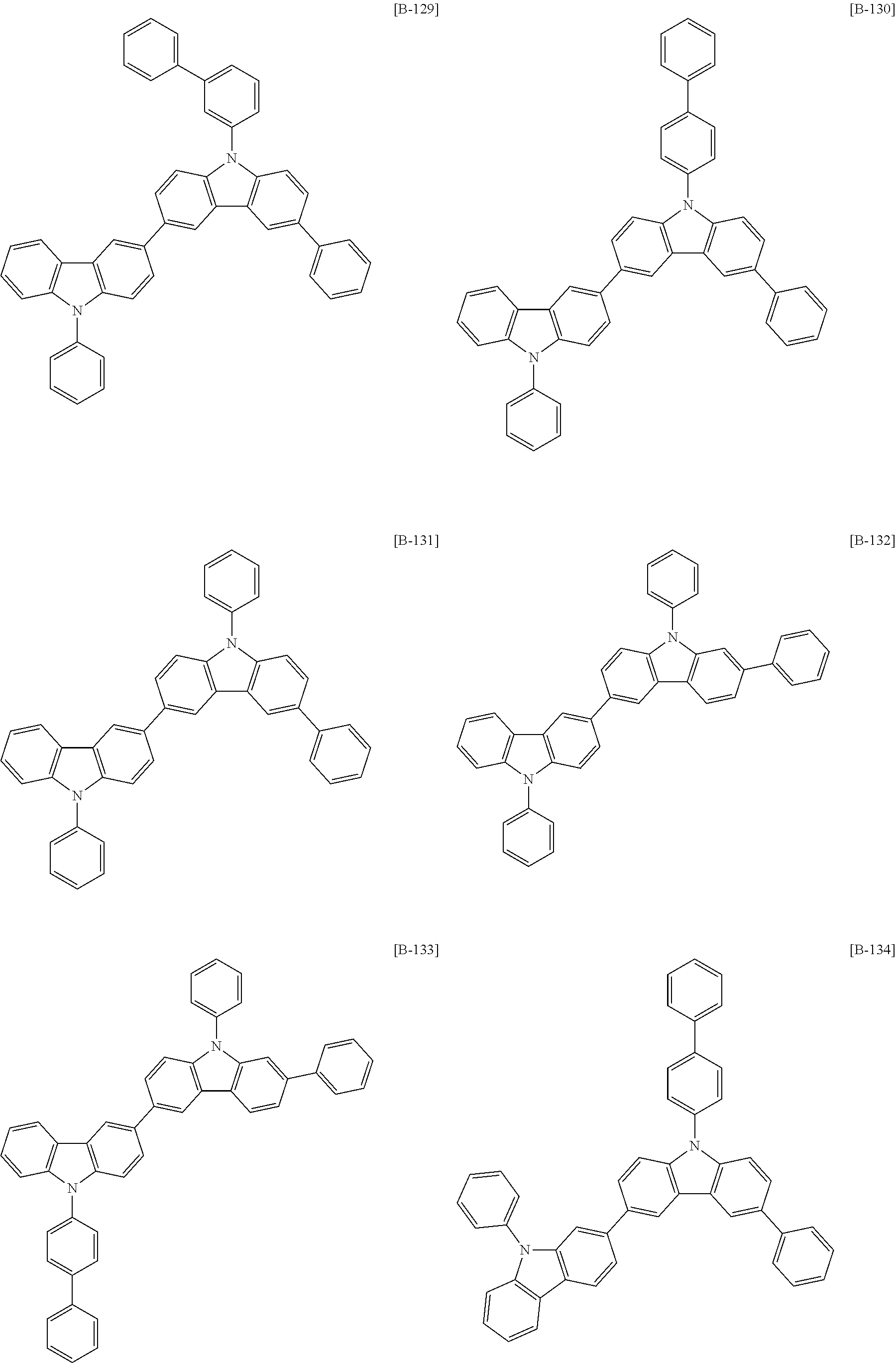

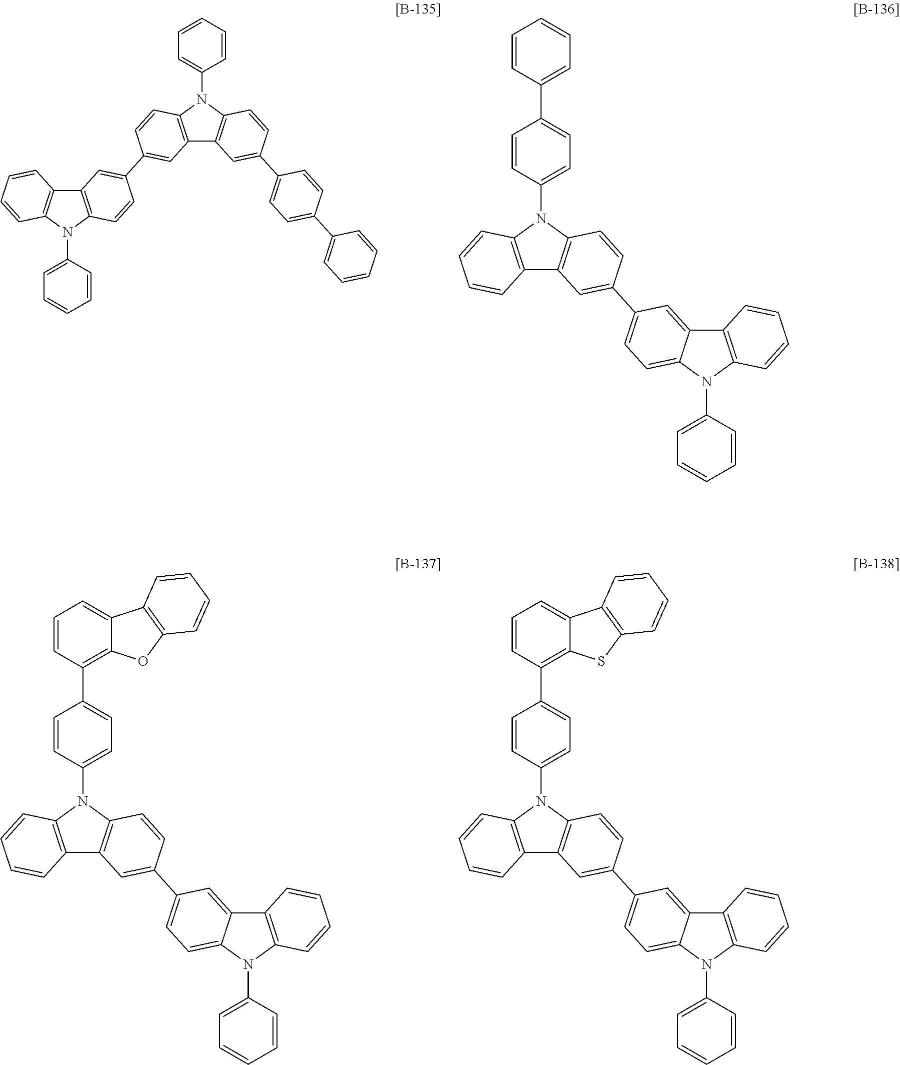 [SMILES: c1ccc(-c2ccc(-c3ccc4c(c3)c3cc(-c5ccc6c(c5)c5ccccc5n6-c5ccccc5)ccc3n4-c3ccccc3)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

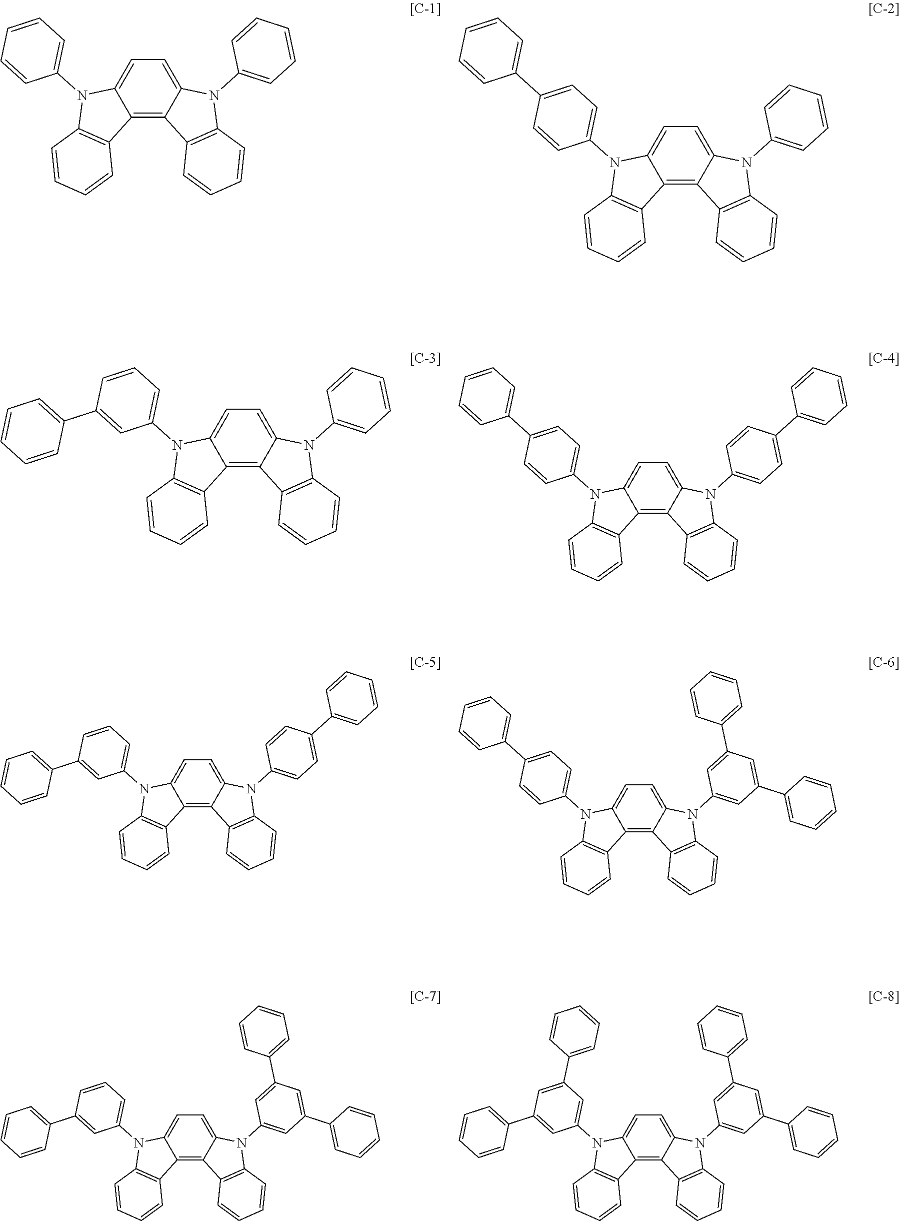

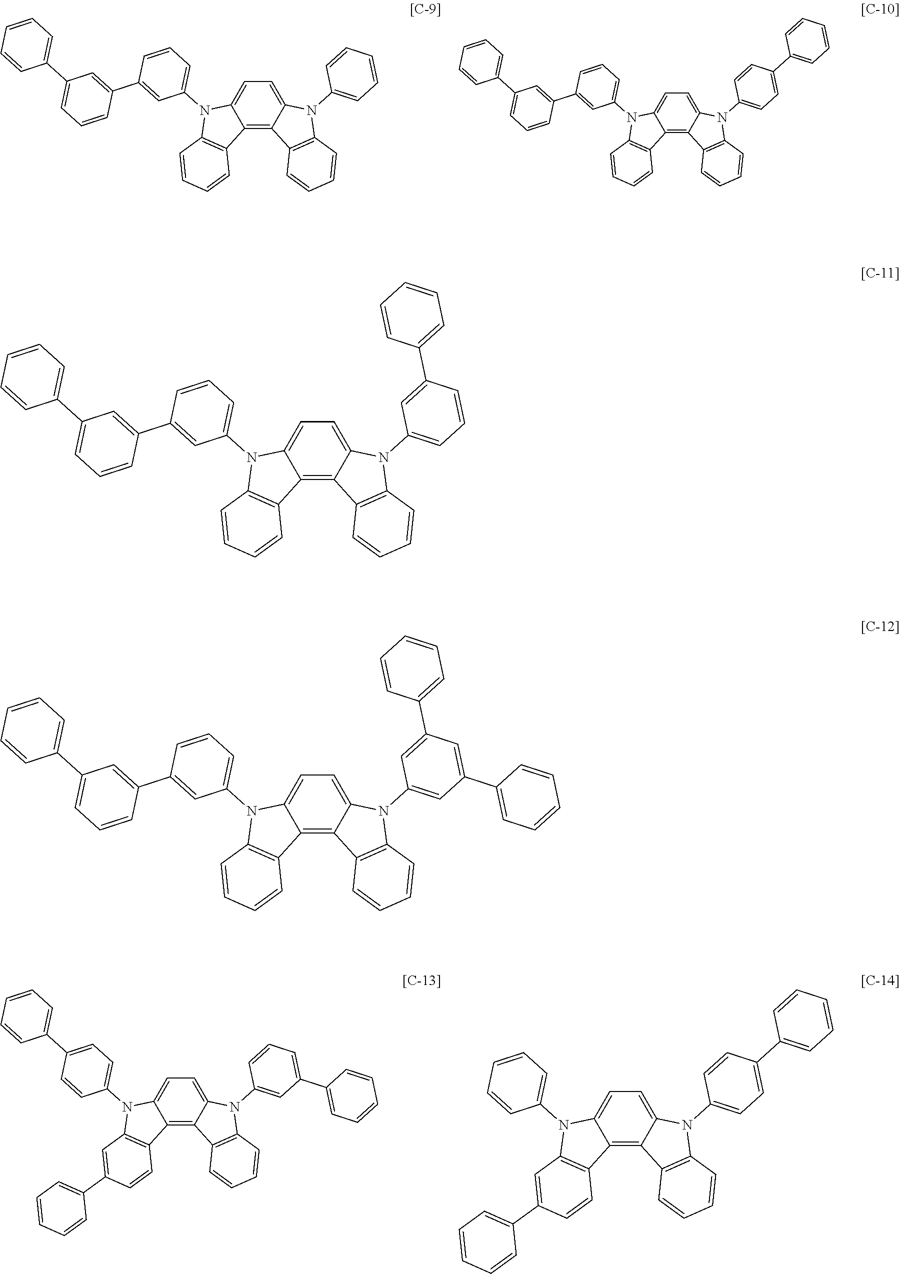

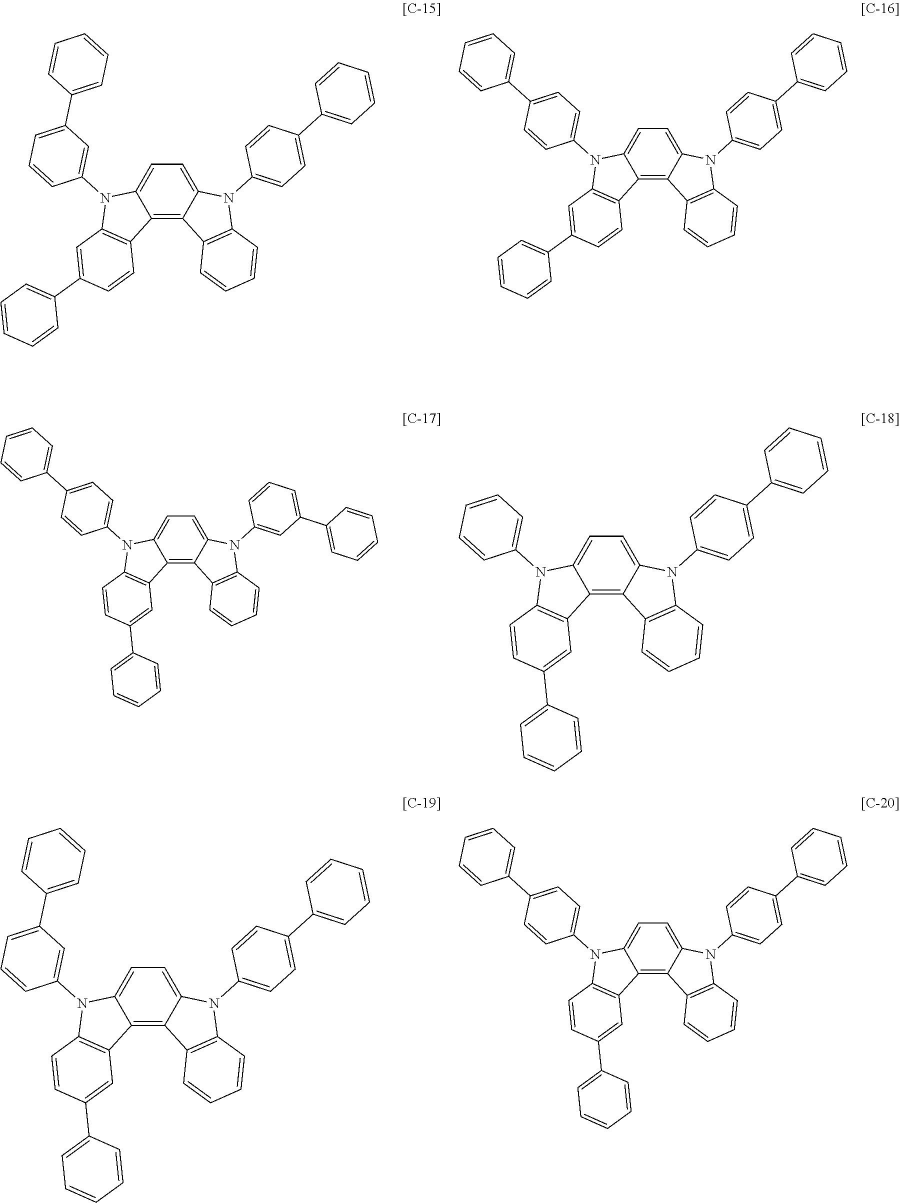

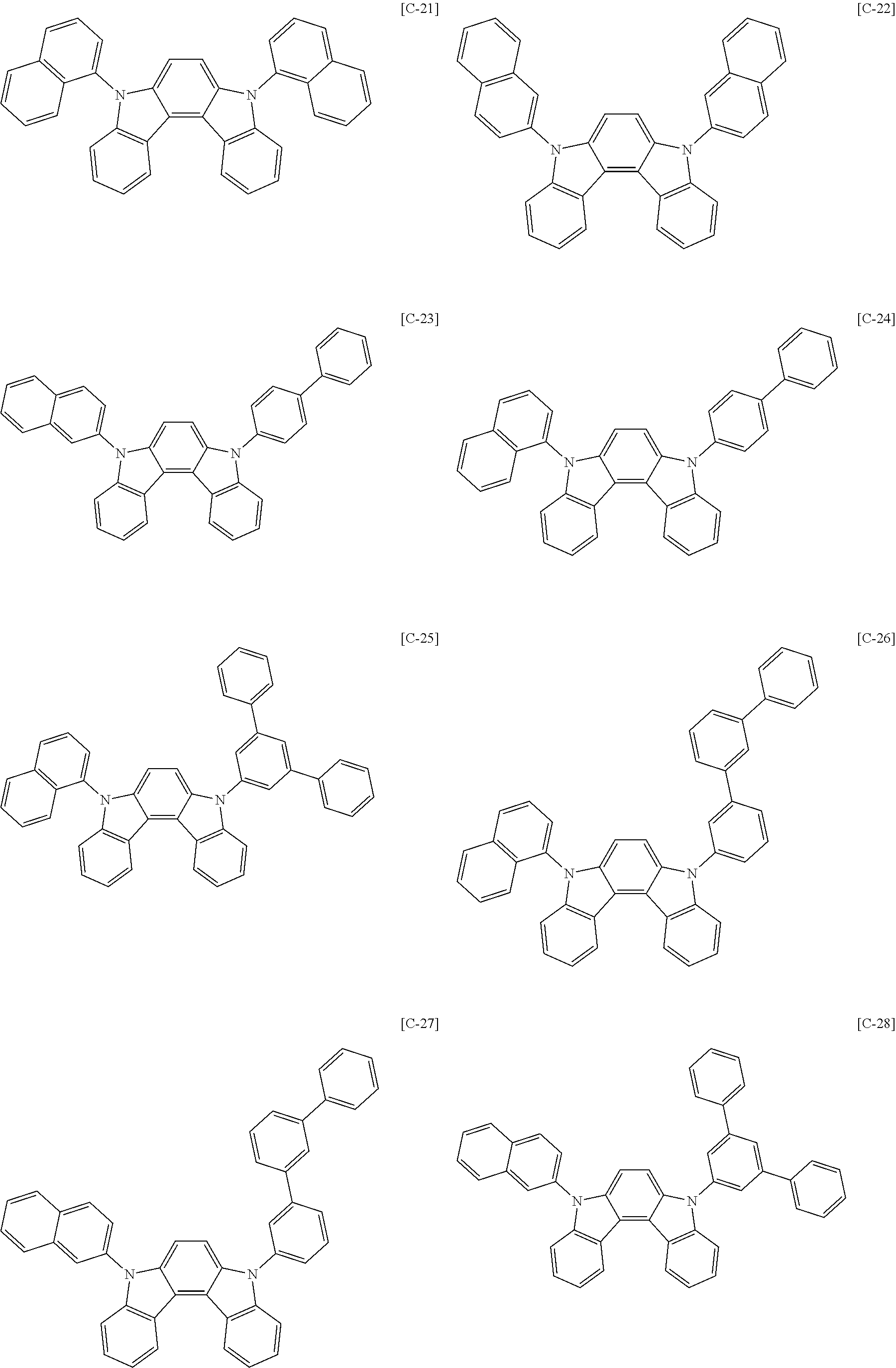

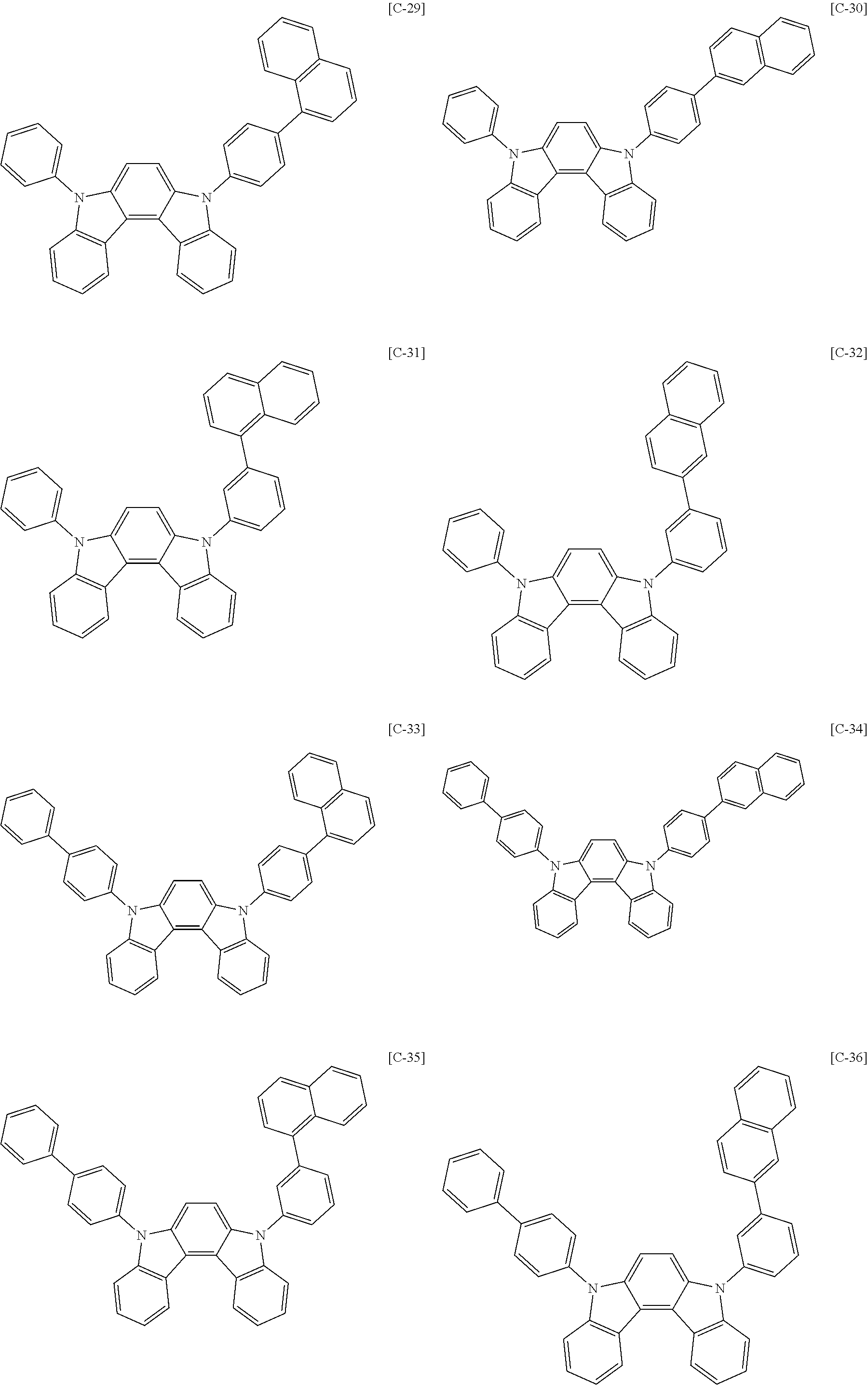

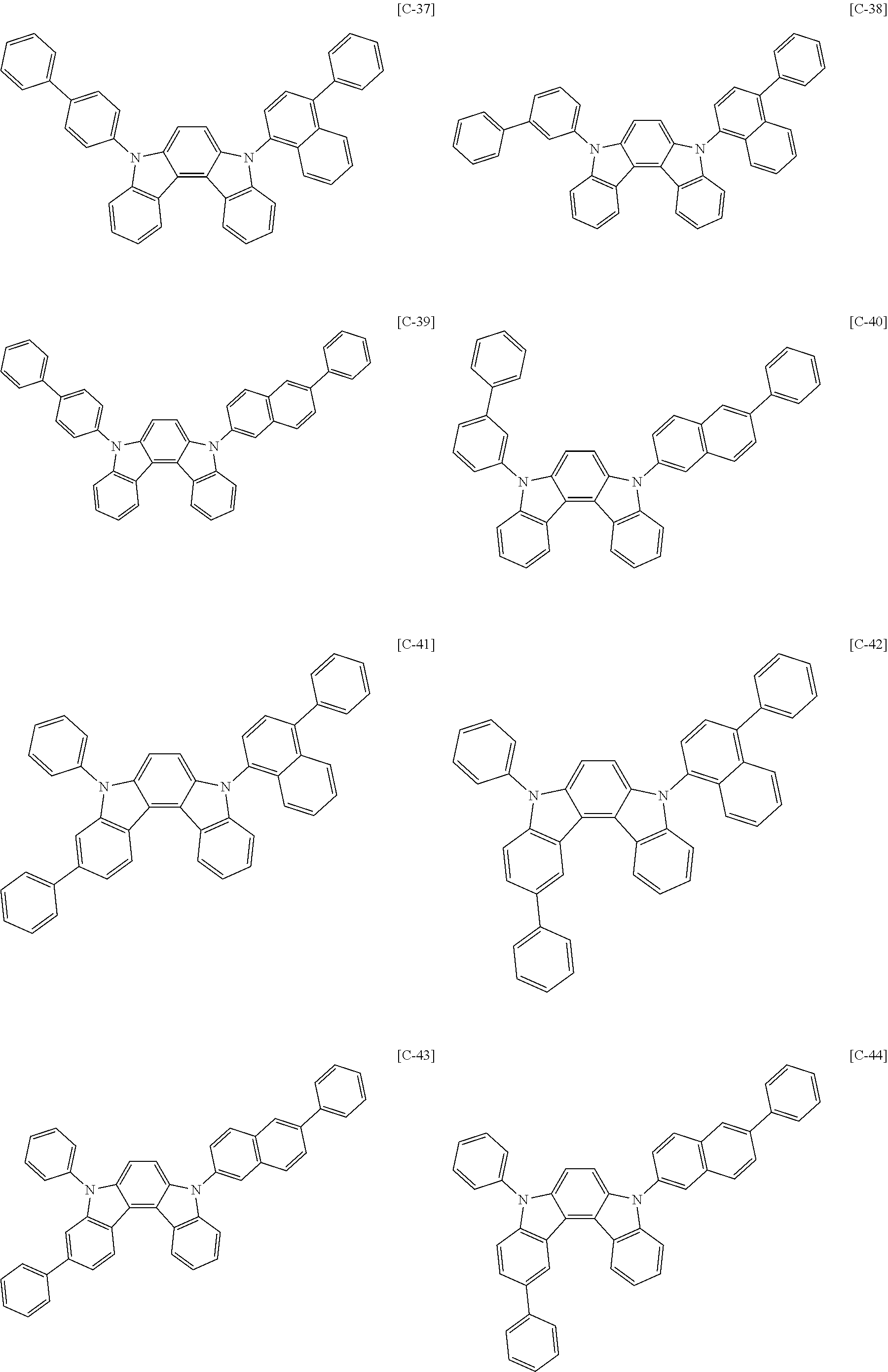

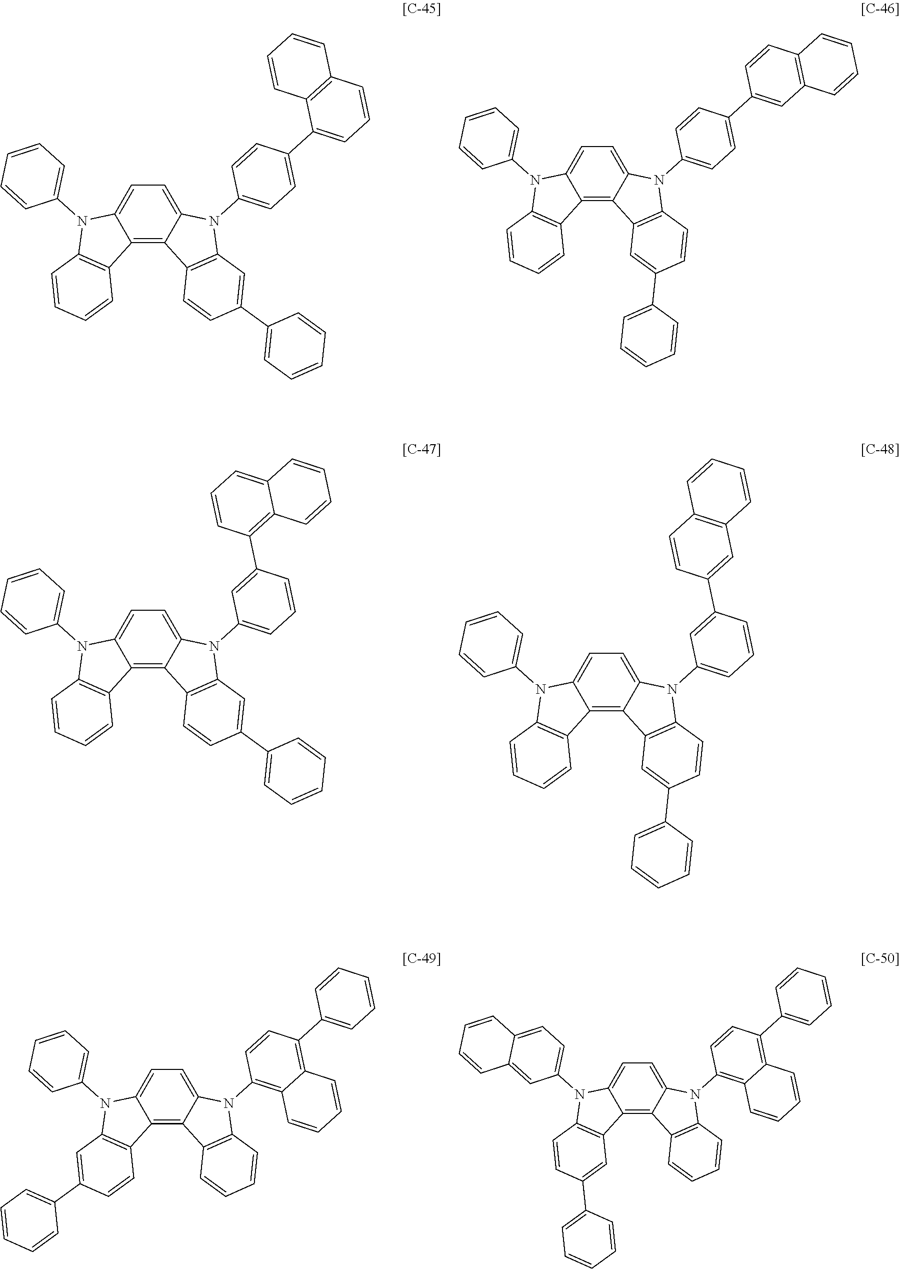

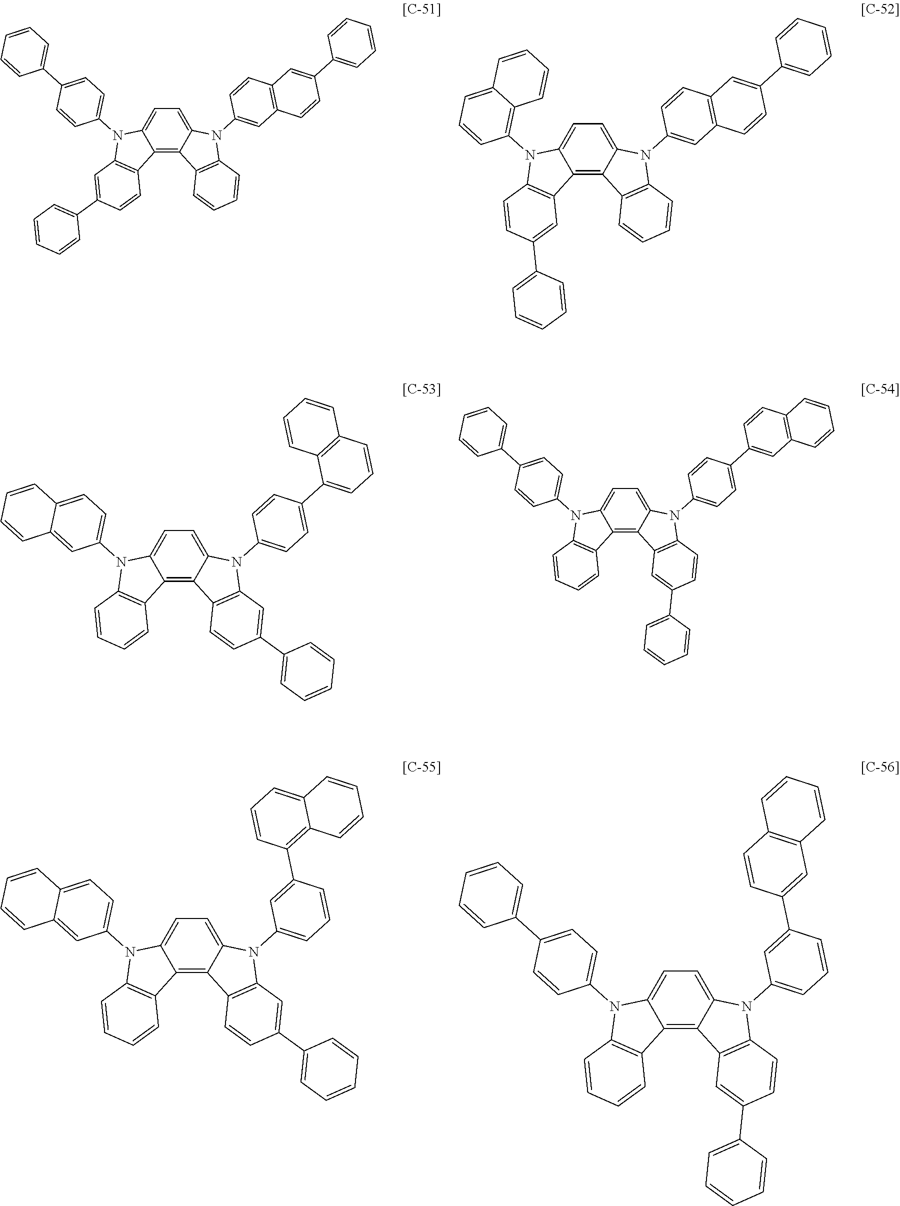

The first compound and the second compound may be included, e.g., mixed, in a weight ratio of about 1:99 to about 99:1. Within the range, a desirable weight ratio may be adjusted using an electron transport capability of the first compound and a hole transport capability of the second compound to realize bipolar characteristics and thus to improve efficiency and life-span. Within the range, they may be, e.g., included in a weight ratio of about 10:90 to 90:10, about 20:80 to 80:20, about 20:80 to about 70:30, about 20:80 to about 60:40, or about 20:80 to about 50:50. Within the range, they may be, e.g., included in a weight ratio of about 20:80 to 40:60, about 30:70, about 40:60, or about 50:50. In an implementation, they may be included in a weight ratio of about 30:70 or about 50:50.

In addition to the aforementioned first compound and second compound, one or more additional compounds may be further included.

The aforementioned compound or composition may be a composition that further includes a dopant.

The dopant may be, e.g., a phosphorescent dopant. In an implementation, the dopant may be, a red, green or blue phosphorescent dopant, e.g., a red or green phosphorescent dopant.

The dopant is a material mixed in a small amount to facilitate light emission, and may be generally a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L^{b}MX^{a} [Chemical Formula Z]

In Chemical Formula Z, M may be a metal, and L^{b} and X^{a} may independently be a ligand to form a complex compound with M.

The M may be, e.g., Ir, Pt, Os, Ti, Zr, Hf, Eu, Tb, Tm, Fe, Co, Ni, Ru, Rh, Pd, or a combination thereof and L^{b} and X^{a} may be, e.g., a bidendate ligand.

The compound or composition may be formed or deposited by a dry film formation method such as chemical vapor deposition (CVD).

Hereinafter, an organic optoelectronic device to which the aforementioned compound or composition applied is described.

The organic optoelectronic device may be any device to convert electrical energy into photoenergy and vice versa without particular limitation, and may be, e.g., an organic photoelectric device, an organic light emitting diode, an organic solar cell, or an organic photoconductor drum.

Herein, an organic light emitting diode as one example of an organic optoelectronic device is described referring to drawings.

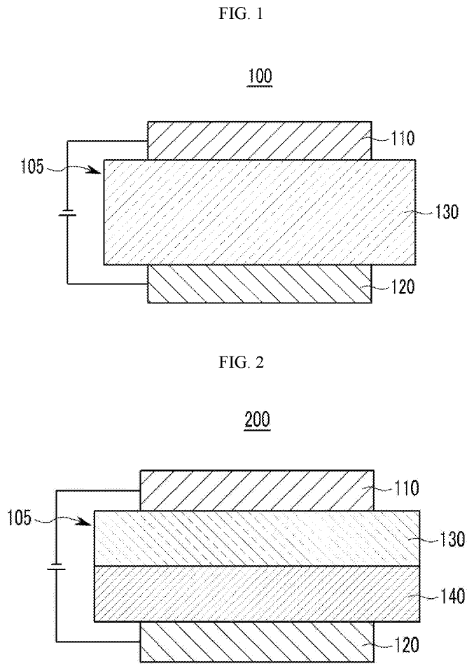

FIGS. 1 and 2 are cross-sectional views showing organic light emitting diodes according to embodiments.

Referring to FIG. 1, an organic light emitting diode 100 according to an embodiment may include an anode 120 and a cathode 110 facing each other and an organic layer 105 disposed between the anode 120 and cathode 110.

The anode 120 may be made of a conductor having a large work function to facilitate hole injection, and may be, e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to facilitate electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; a multi-layer structure material such as LiF/Al, LiO₂/Al, LiF/Ca, LiF/Al, or BaF₂/Ca.

The organic layer 105 may include a light emitting layer 130 that includes the aforementioned compound or composition.

The light emitting layer 130 may include, e.g., the aforementioned compound or composition.

Referring to FIG. 2, an organic light emitting diode 200 may further include a hole auxiliary layer 140 in addition to the light emitting layer 130. The hole auxiliary layer 140 further increases hole injection and/or hole mobility and blocks electrons between the anode 120 and the light emitting layer 130. The hole auxiliary layer 140 may be, e.g., a hole transport layer, a hole injection layer, or an electron blocking layer, and may include at least one layer.

The hole auxiliary layer 140 may include e.g., a compound of the following Group A.

In an implementation, the hole auxiliary layer 140 may include a hole transport layer between the anode 120 and the light emitting layer 130 and a hole transport auxiliary layer between the light emitting layer 130 and the hole transport layer, and a compound of Group A may be included in the hole transport auxiliary layer.

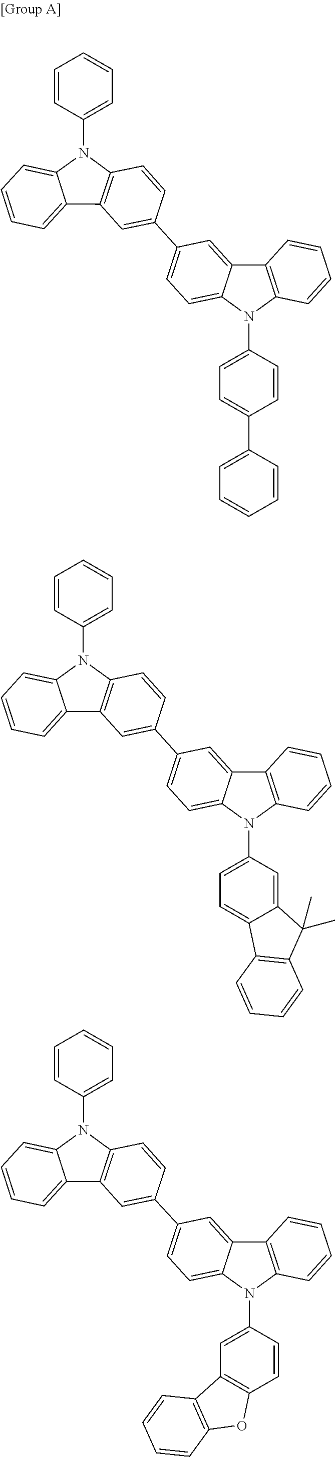 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

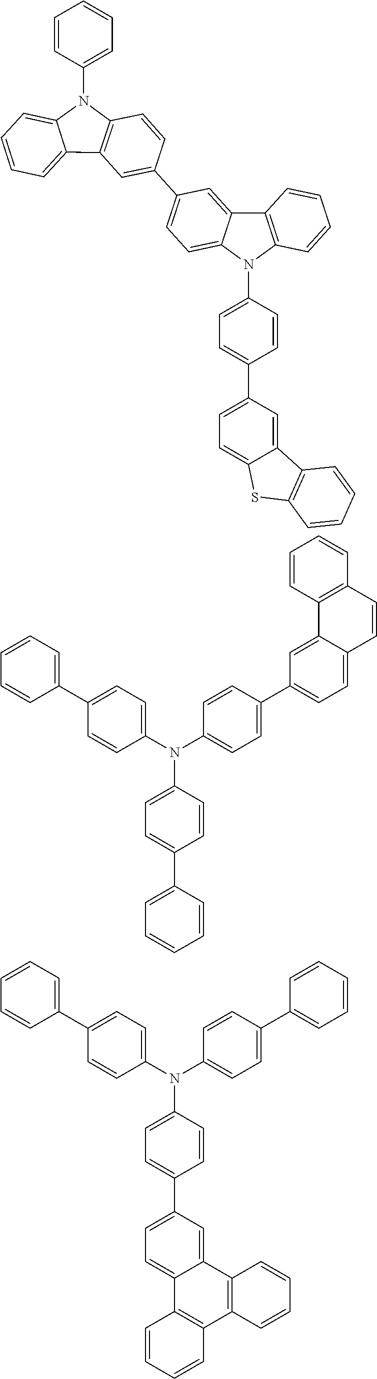

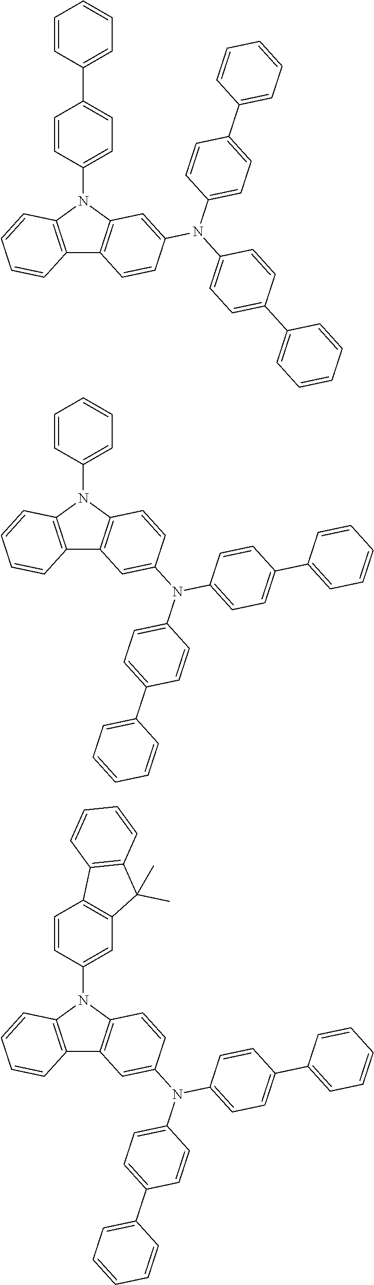

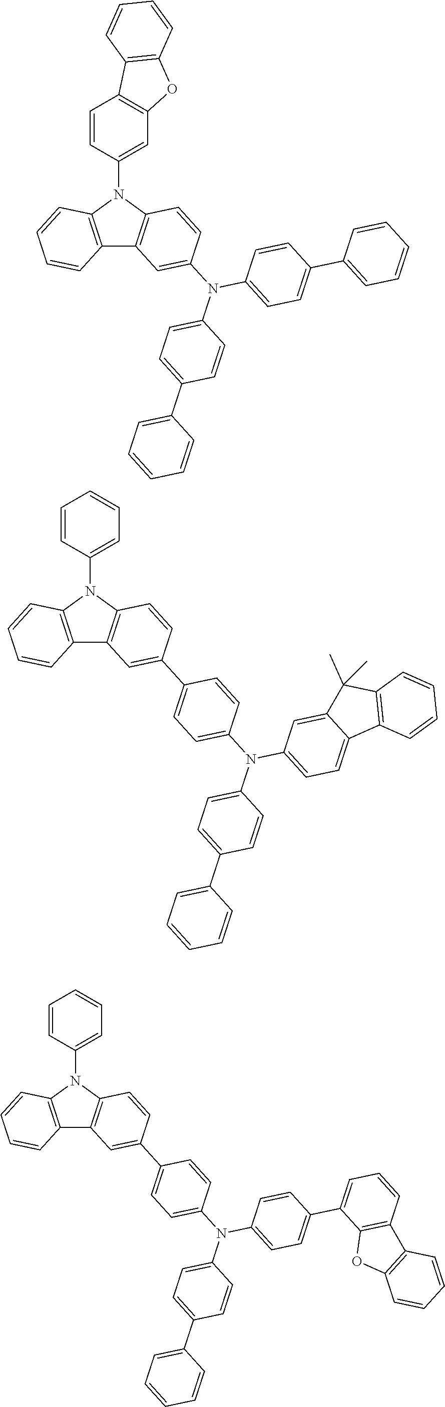

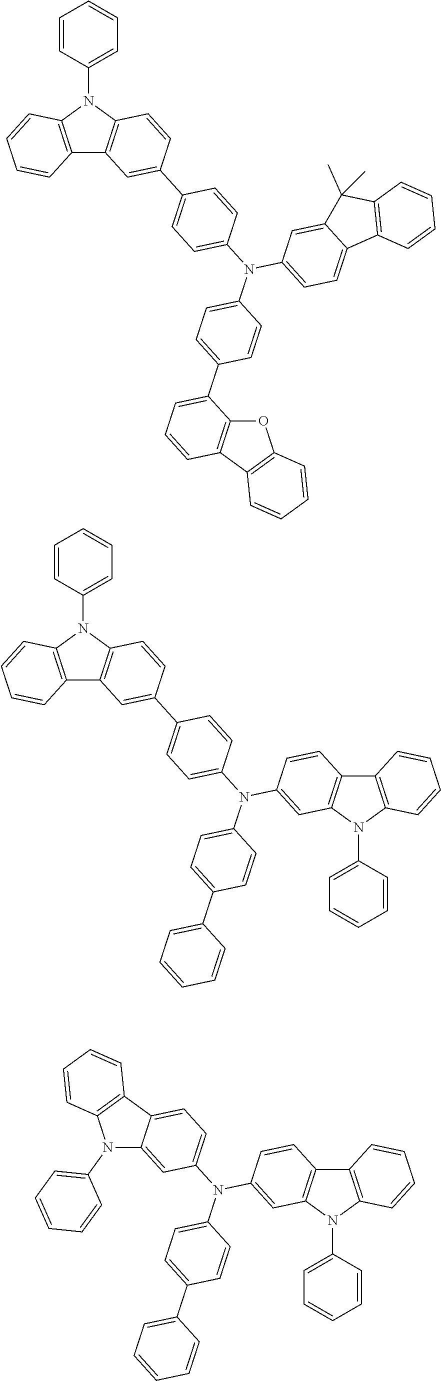 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc4c5ccccc5n(-c5ccccc5)c4c3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1]

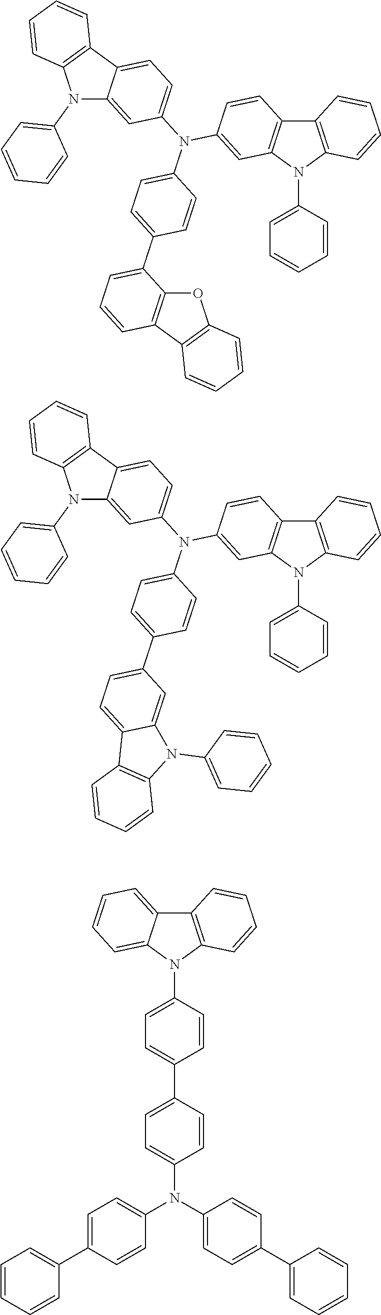 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

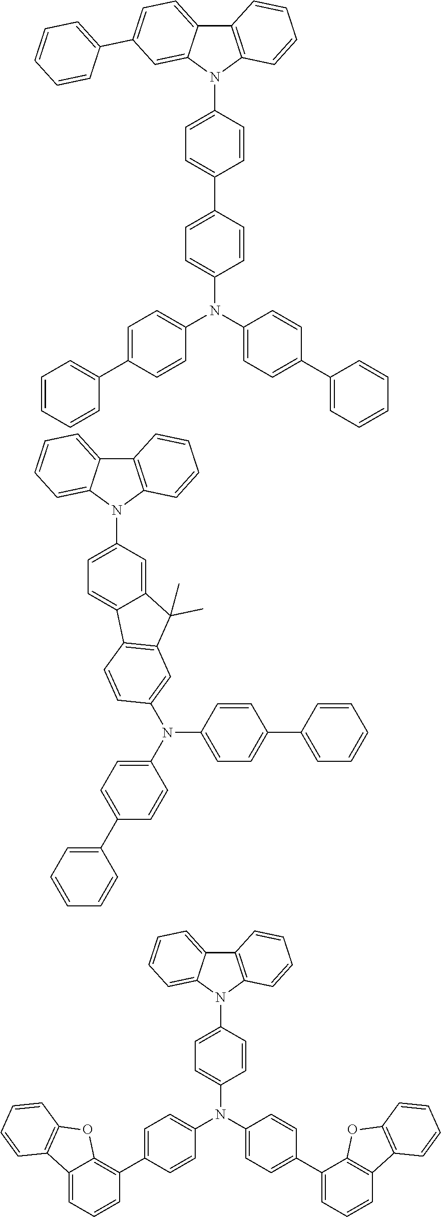

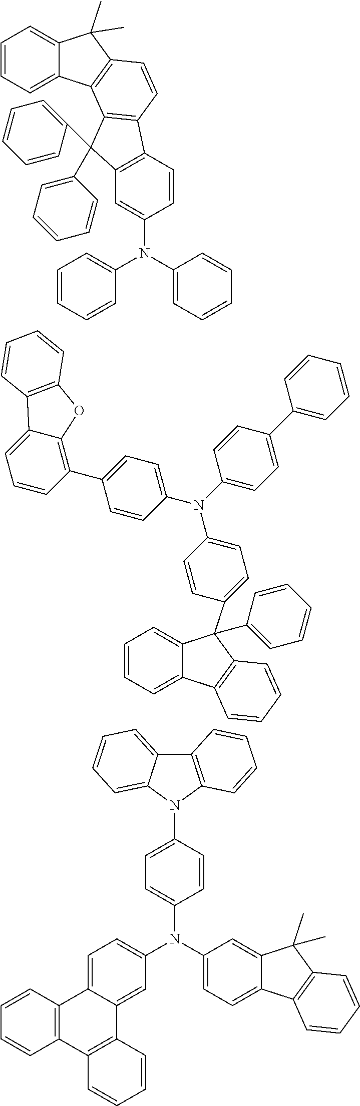

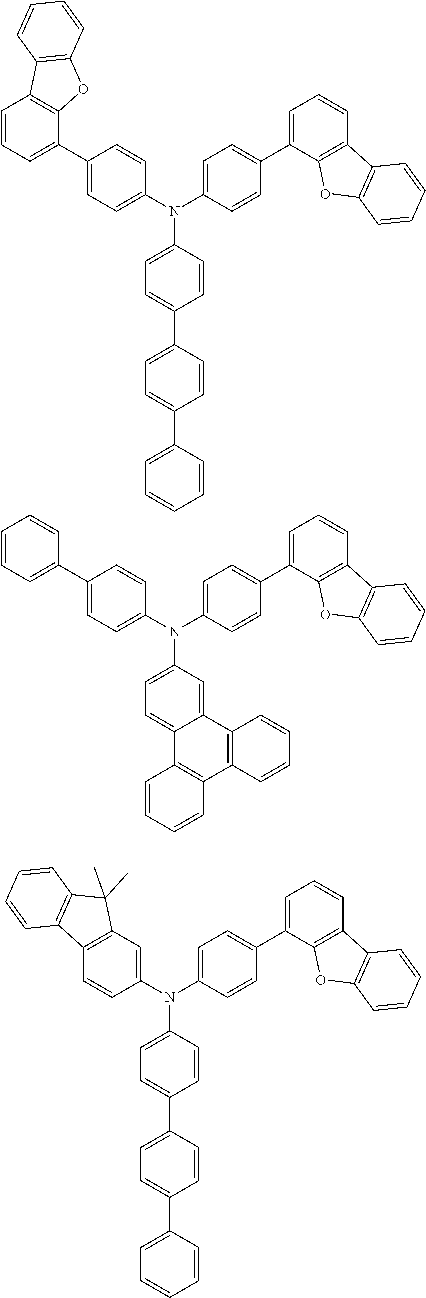 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

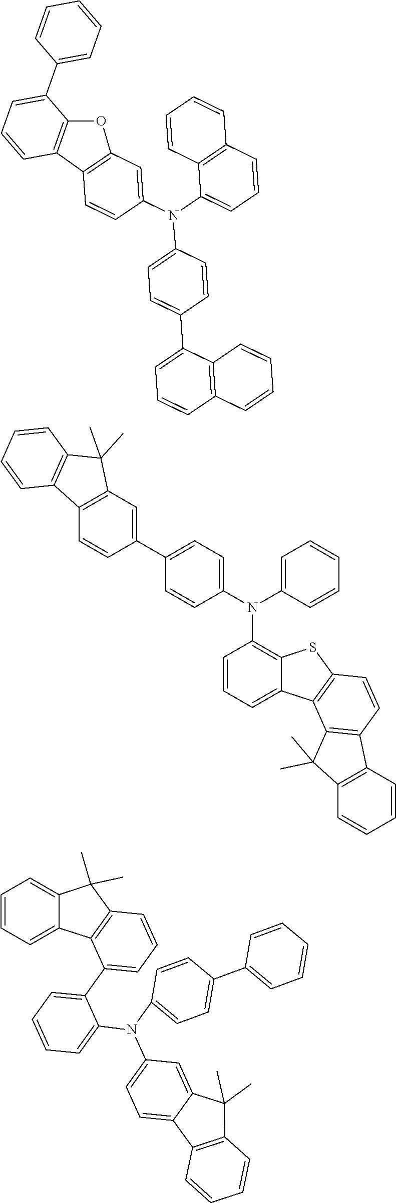 [SMILES: c1ccc(-c2cccc3c2oc2cc(N(c4ccc(-c5cccc6ccccc56)cc4)c4cccc5ccccc45)ccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

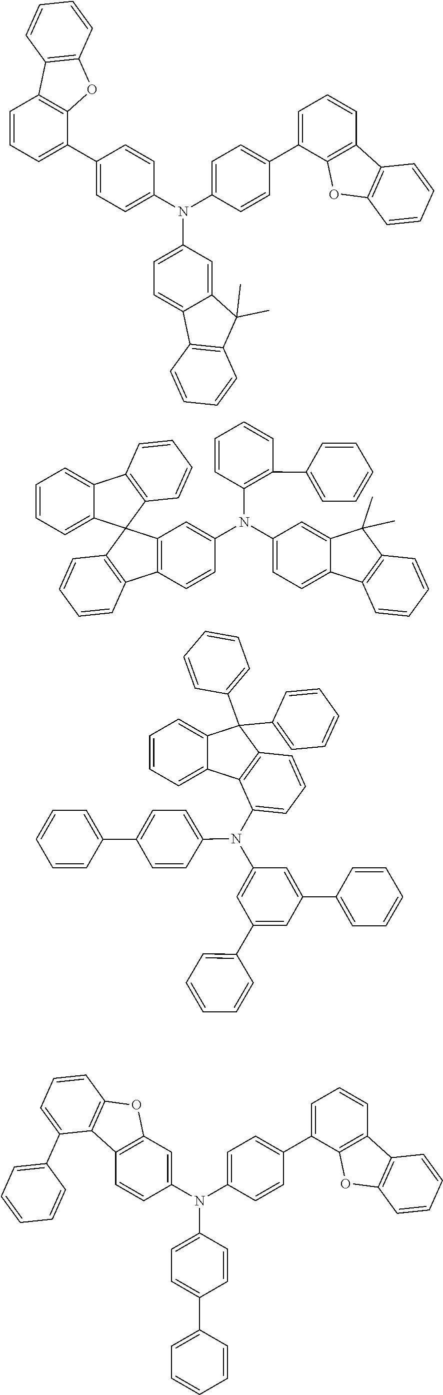

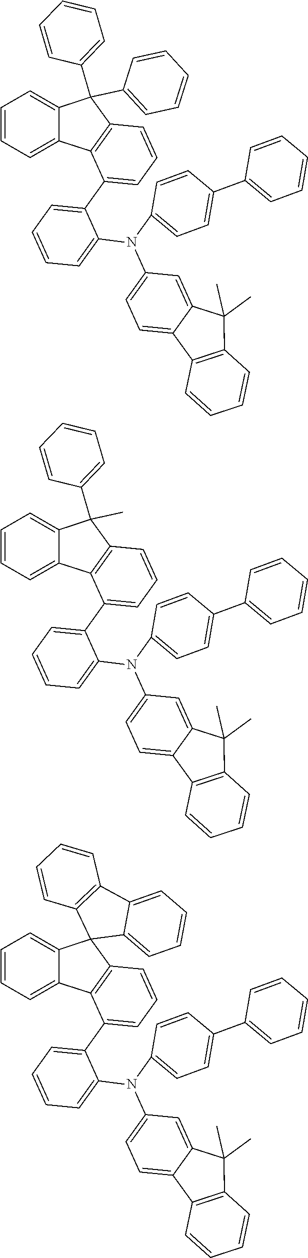 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

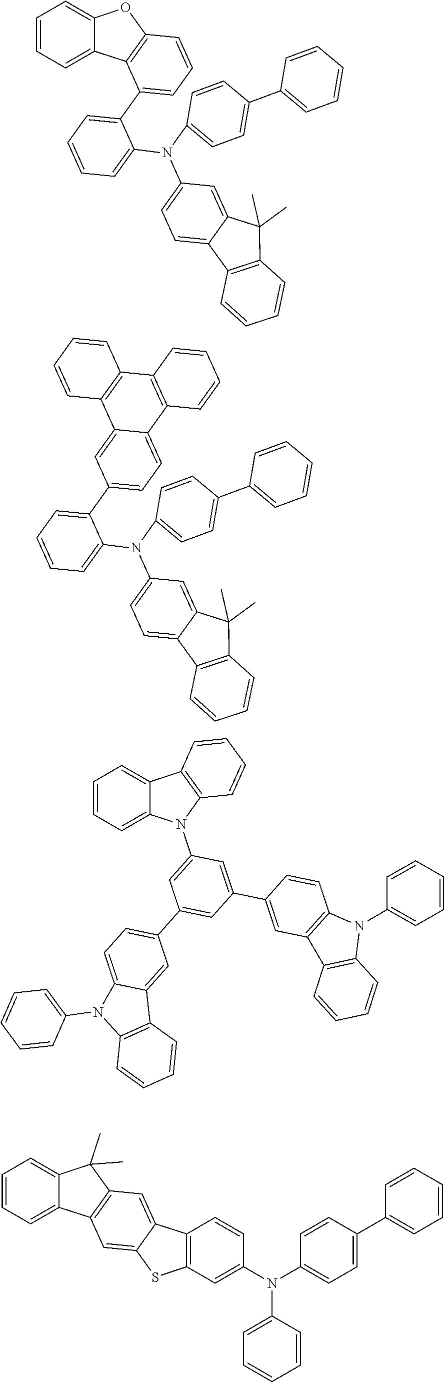

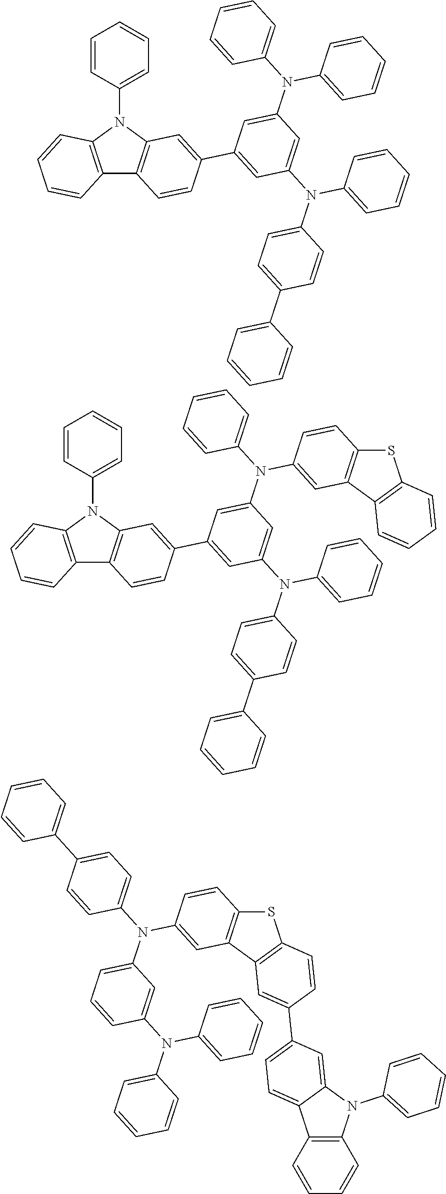

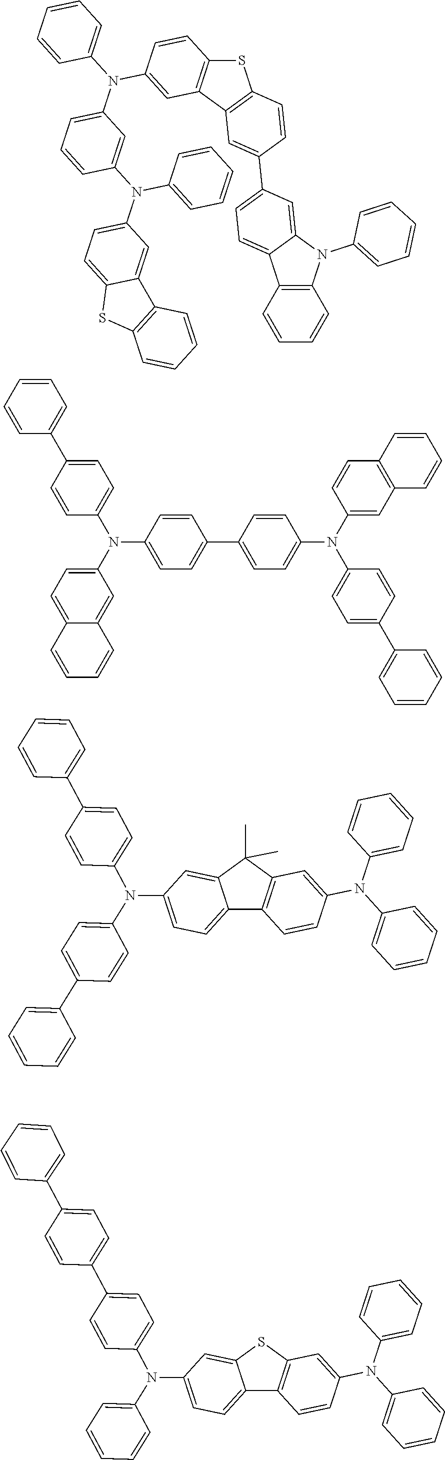

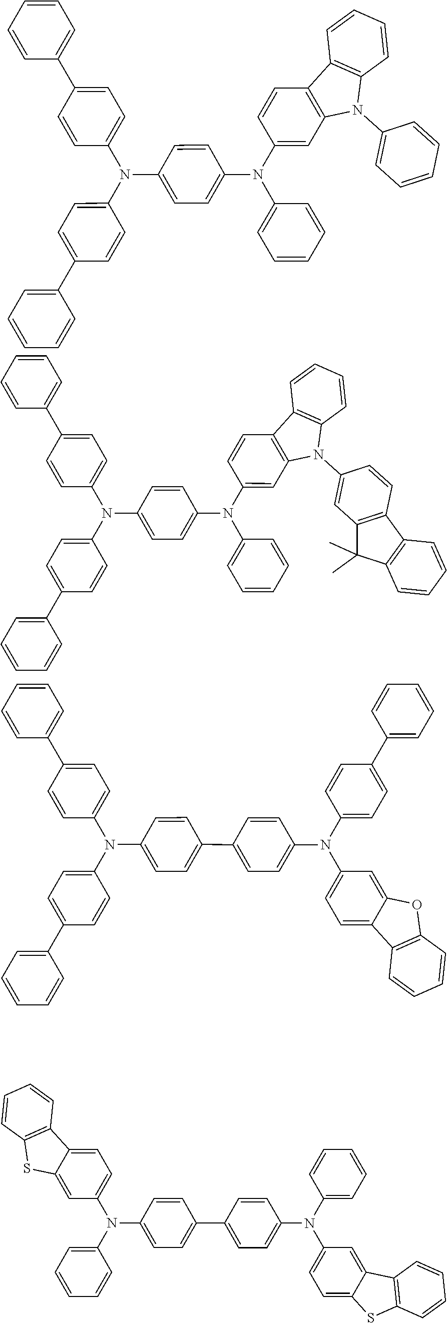

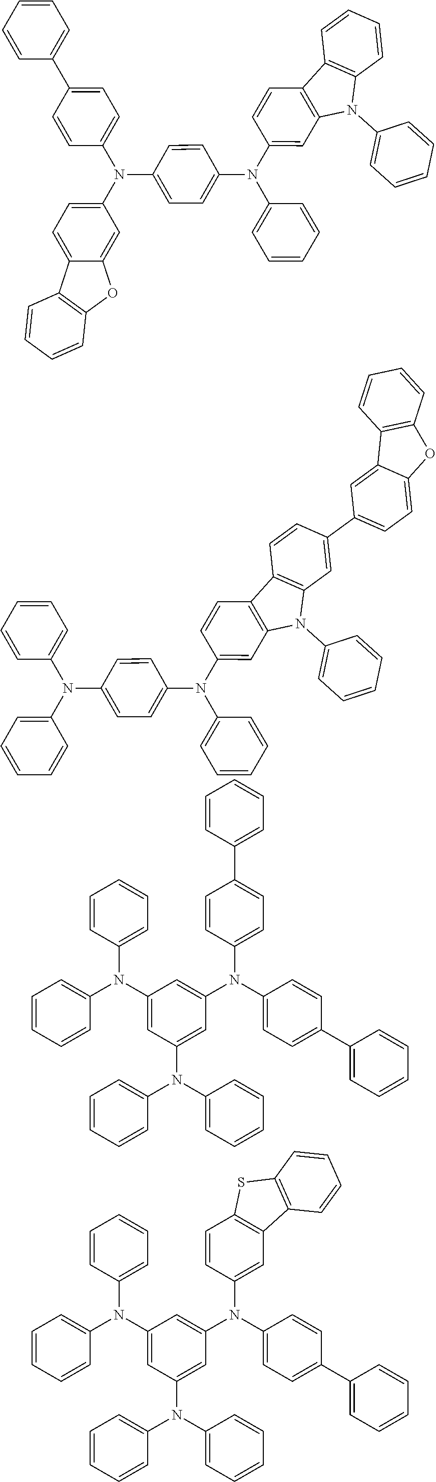

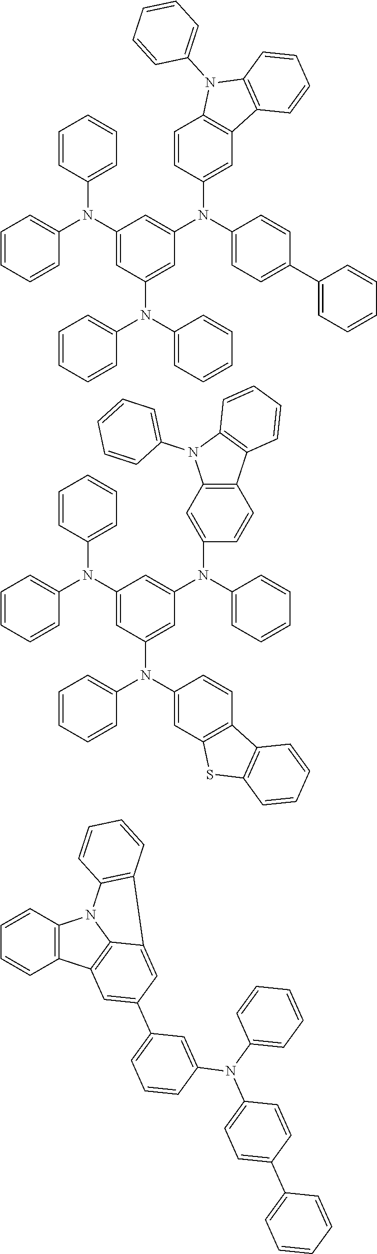

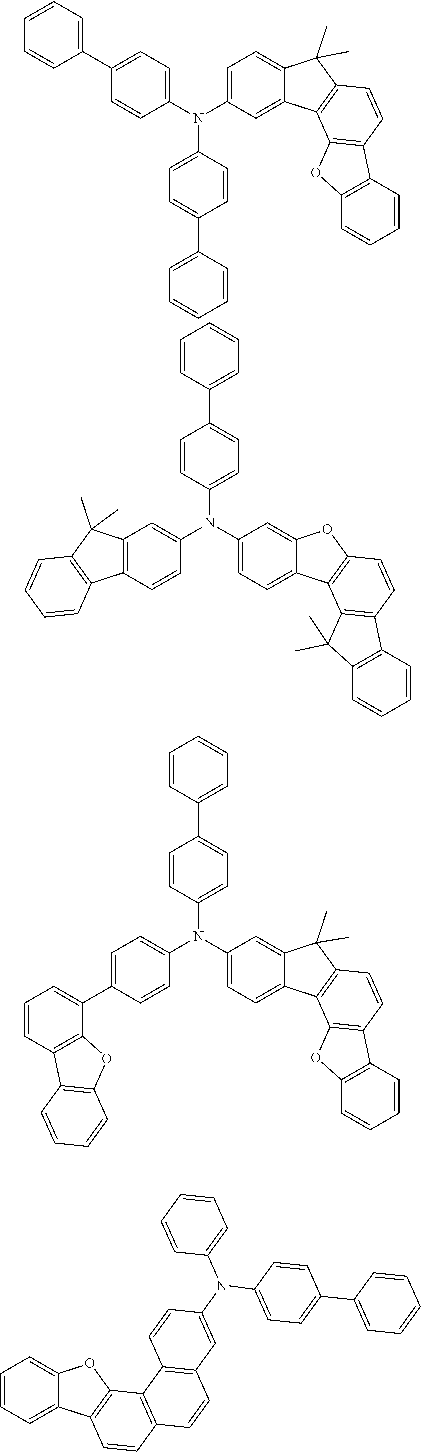

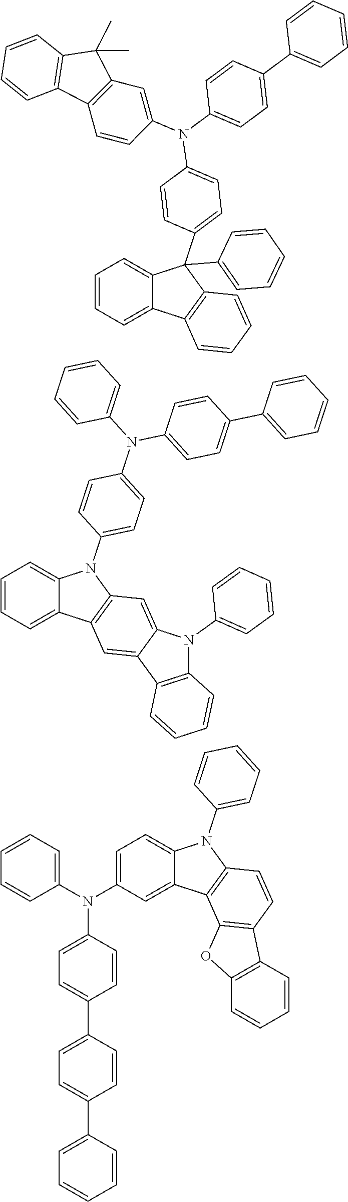

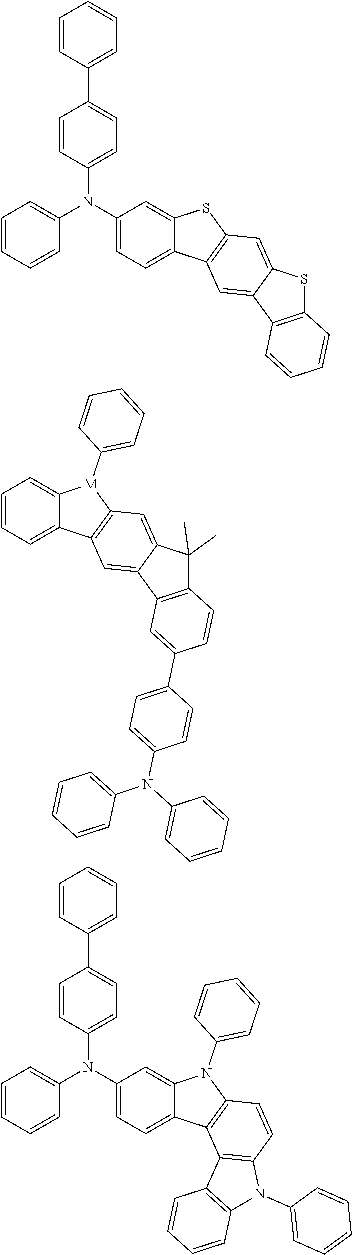

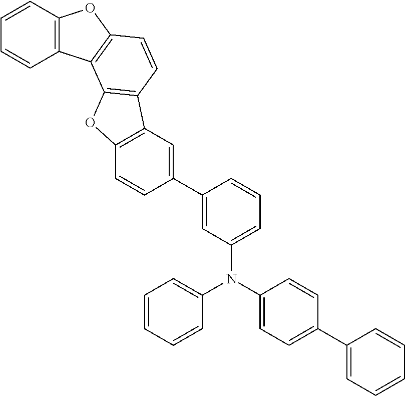 [SMILES: c1ccc(-c2ccc(N(c3ccccc3)c3cccc(-c4ccc5oc6c(ccc7oc8ccccc8c76)c5c4)c3)cc2)cc1]

In the hole transport auxiliary layer, other suitable compounds may be used in addition to the compound.

In an implementation, an organic light emitting diode may further include an electron transport layer, an electron injection layer, or a hole injection layer as the organic layer 105.

The organic light emitting diodes 100 and 200 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry or P&H tech as far as there in no particular comment or were synthesized by suitable methods.

### Preparation of Compound for Organic Optoelectronic Device

### Synthesis Example 1: Synthesis of Intermediate B

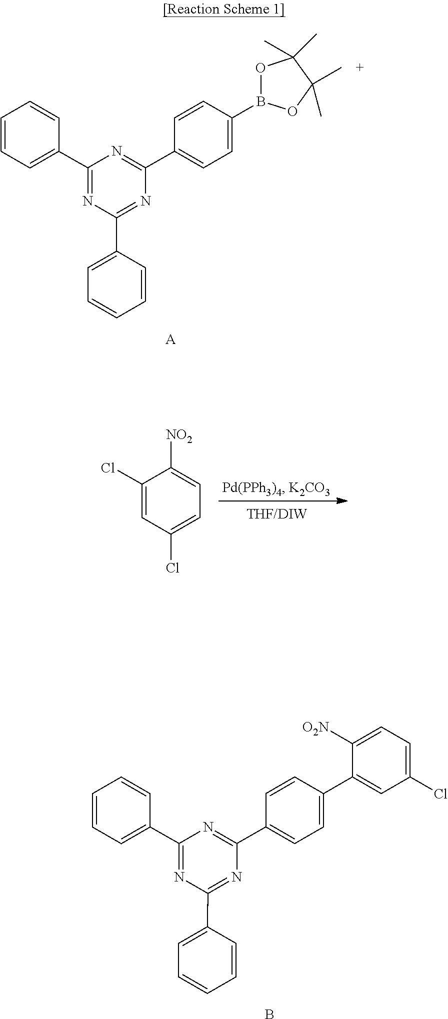

13.8 g (22.97 mmol) of Intermediate A, 4.41 g (22.97 mmol) of 2,4-dichloro-nitobenzene, 0.8 g (0.69 mmol) of Pd(PPh₃)₄, and 6.35 g (45.94 mmol) of K₂CO₃ were dissolved in 100 ml of THF and 55 ml of DIW and then, refluxed and stirred for 12 hours. When a reaction was complete, a solid produced therein was filtered and then, heated and dissolved in 400 ml of monochlorobenzene and filtered with silica gel. A solid produced from the filtrate was dried to obtain 6.5 g (61%) of Intermediate B as a target compound.

### Synthesis Example 2: Synthesis of Intermediate C

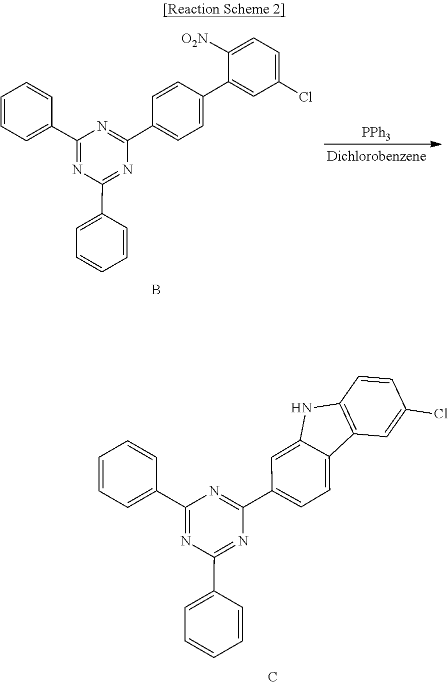

6.5 g (13.98 mmol) of Intermediate B and 9.17 g (34.95 mmol) of PPh₃ were suspended in 50 ml of dichlorobenzene and then, refluxed and stirred for 24 hours. When a reaction was complete, the resultant was silica gel-columned to obtain 2.6 g (43%) of Intermediate C as a target compound.

### Synthesis Example 3: Synthesis of Intermediate D

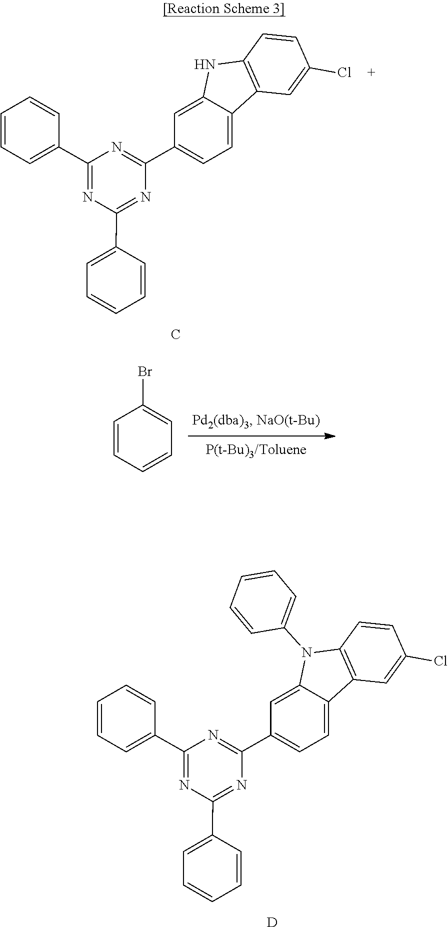

2.6 g (6.00 mmol) of Intermediate C, 1.23 g (7.81 mmol) of bromobenzene, 0.3 g (0.3 mmol) of Pd₂(dba)₃, 1.15 g (12.01 mmol) of NaO(t-Bu), and 0.12 g (0.60 mmol) of P(t-Bu)₃ were suspended in 50 ml of toluene and then, refluxed and stirred for 12 hours. When a reaction was complete, the resultant was cooled down to ambient temperature, distilled water was added thereto, and an organic layer was extracted therefrom, concentrated, and silica gel-columned to obtain 2.6 g (86%) of Intermediate D as a target compound.

### Synthesis Example 4: Synthesis of Compound 5

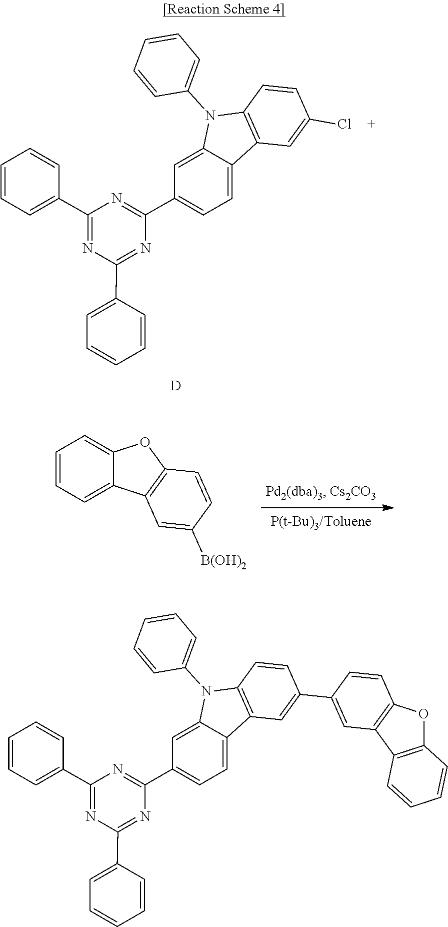

2.5 g (64%) of Compound 5 as a target compound was obtained according to the same method as Synthesis Example 1 except that 3.14 g (6.18 mmol) of Intermediate D, 1.44 g (6.78 mmol) of 2-dibenzofuranylboronic acid, 0.17 g (0.18 mmol) of Pd₂(dba)₃, 4.02 g (12.34 mmol) of Cs₂CO₃, and 0.12 g (0.61 mmol) of P(t-Bu)₃ were used.

(LC/MS: theoretical value 640.73, measured value: 641.2)

### Synthesis Example 5: Synthesis of Compound 6

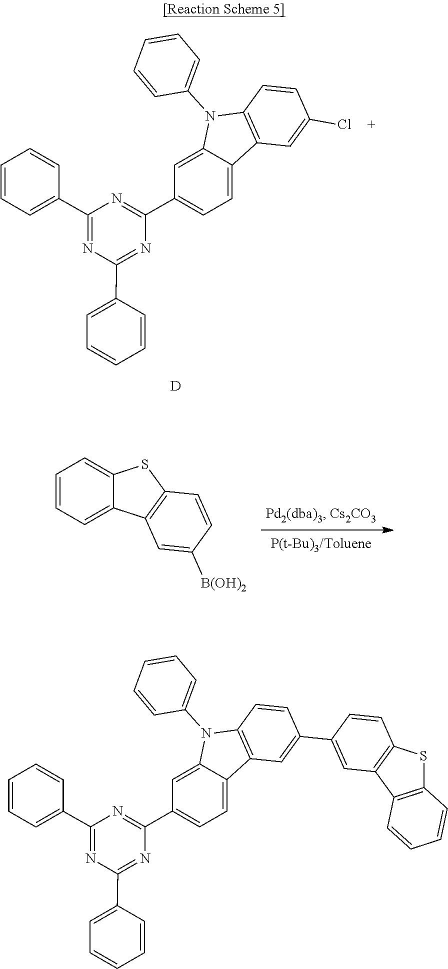

2.3 g (56%) of Compound 6 as a target compound was obtained according to the same method as Synthesis Example 4 except that 3.14 g (6.18 mmol) of Intermediate D and 1.54 g of 2-dibenzothiopheneboronic acid were used.

(LC/MS: theoretical value 656.8, measured value: 657.4)

### Synthesis Example 6: Synthesis of Comparative Compound 1

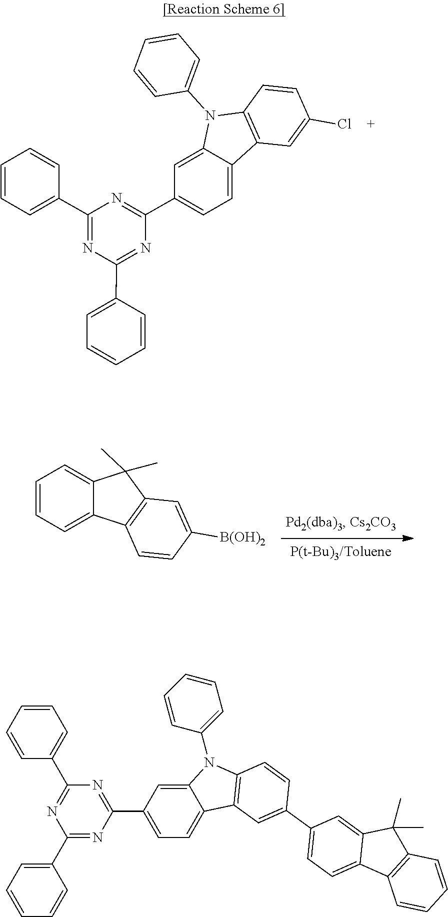 [SMILES: Clc1ccc2c(c1)c1ccc(-c3nc(-c4ccccc4)nc(-c4ccccc4)n3)cc1n2-c1ccccc1 ; CC1(C)c2ccccc2-c2ccc(B(O)O)cc21 ; CC1(C)c2ccccc2-c2ccc(-c3ccc4c(c3)c3ccc(-c5nc(-c6ccccc6)nc(-c6ccccc6)n5)cc3n4-c3ccccc3)cc21]

2.6 g (64%) of Comparative Compound 1 as a target compound was obtained according to the same method as Synthesis Example 4 except that 3.14 g (6.18 mmol) of Intermediate D and 1.61 g of 2-fluoroeneboronic acid were used.

(LC/MS: theoretical value 656.8, measured value: 667.4)

(Manufacture of Organic Light Emitting Diode)

### Example 1

A glass substrate coated with ITO (indium tin oxide) with a thickness of 1,500 Å was washed with distilled water. After washing with the distilled water, the glass substrate was ultrasonically washed with isopropyl alcohol, acetone, or methanol, dried, and then moved to a plasma cleaner, cleaned using oxygen plasma for 10 minutes, and moved to a vacuum depositor. This obtained ITO transparent electrode was used as an anode, Compound A was vacuum-deposited on the ITO substrate to form a 700 Å-thick hole injection layer, and Compound B was deposited to be 50 Å-thick on the injection layer, and then Compound C was deposited to be 1,020 Å-thick to form a hole transport layer. On the hole transport layer, 400 Å-thick light emitting layer was formed by using Compound 5 and Compound B-99 simultaneously as a host and doping 10 wt % of tris(2-phenylpyridine)iridium(III) [Ir(ppy)₃] as a dopant by a vacuum-deposition. Herein, Compound 5 and Compound B-99 were used in a weight ratio of 3:7. Subsequently, on the light emitting layer, a 300 Å-thick electron transport layer was formed by simultaneously vacuum-depositing Compound D and Liq in a weight ratio of 1:1, and on the electron transport layer, Liq and Al were sequentially vacuum-deposited to be 15 Å-thick and 1,200 Å-thick, manufacturing an organic light emitting diode.

The organic light emitting diode had a five-layered organic thin layer, and specifically the following structure.

ITO/Compound A (700 Å)/Compound B (50 Å)/Compound C (1,020 Å)/EML [90 wt % of host (Compound 5 and Compound B-99 were mixed in a weight ratio of 3:7) and 10 wt % of Ir(ppy)₃] (400 Å)/Compound D:Liq (300 Å)/Liq (15 Å)/Al (1,200 Å)

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN),

Compound C: N-(biphenyl-4-yl)-9,9-dimethyl-N-(4-(9-phenyl-9H-carbazol-3-yl)phenyl)-9H-fluoren-2-amine

Compound D: 8-(4-(4,6-di(naphthalen-2-yl)-1,3,5-triazin-2-yl)phenyl)quinolone

### Example 2

An organic light emitting diode was manufactured in the same manner as in Example 1, except that Compound 6 was used instead of Compound 5.

### Comparative Example 1

An organic light emitting diode was manufactured in the same manner as in Example 1, except that Comparative Compound 1 was used instead of Compound 5.

Evaluation

Driving voltages, luminous efficiency, and life-span characteristics of the organic light emitting diodes according to Examples 1 and 2 and Comparative Example 1 were evaluated.

Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Current Efficiency

Current efficiency (cd/A) at the same current density (10 mA/cm²) were calculated by using the luminance and current density from the items (1) and (2), and a voltage.

(4) Measurement of Life-Span

The results were obtained by measuring a time when current efficiency (cd/A) was decreased down to 90%, while luminance (cd/m²) was maintained to be 6,000 cd/m².

(5) Measurement of Driving Voltage

A driving voltage of each diode was measured using a current-voltage meter (Keithley 2400) at 15 mA/cm².

(6) Calculation of Life-span Ratio

T90 life-span ratios of Example 1 and Example 2 relative to the T90 life-span of Comparative Example 1 were calculated and shown in Table 1.

(7) Calculation of Driving Voltage Ratio

Driving voltages of Examples 1 and 2 relative to the driving voltage of Comparative Example 1 were calculated and shown in Table 1.

TABLE 1

| | | | Driving |
|---|---|---|---|
| | | T90 life- | voltage |
| | | span ratio | ratio |
| | Host | (%) | (%) |
| Example 1 | Compound 5 | 160% | 96% |
| Example 2 | Compound 6 | 140% | 95% |
| Comparative | Comparative | 100% | 100% |
| Example 1 | Compound 1 | | |

Referring to Table 1, the driving voltages of the organic light emitting diodes according to Examples 1 and 2 were improved, compared with the organic light emitting diode according to Comparative Example 1. For example, the life-span characteristics were remarkably improved.

One or more embodiments may provide a compound for an organic optoelectronic device capable of implementing a high efficiency and long life-span organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 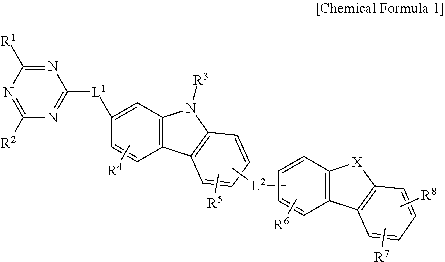
wherein, in Chemical Formula 1,
X is O or S,
L¹ and L² are independently a single bond or a substituted or unsubstituted C6 to C20 arylene group,
R¹ to R³ are independently a substituted or unsubstituted C6 to C30 aryl group or a substituted or unsubstituted C2 to C30 heterocyclic group,
R⁴ to R⁸ are independently hydrogen, deuterium, a cyano group, a halogen, a substituted or unsubstituted C1 to C30 alkyl group, or a substituted or unsubstituted C6 to C30 aryl group.

2. The compound as claimed in claim 1, wherein the compound represented by Chemical Formula 1 is represented by one of Chemical Formula 1A to Chemical Formula 1D: 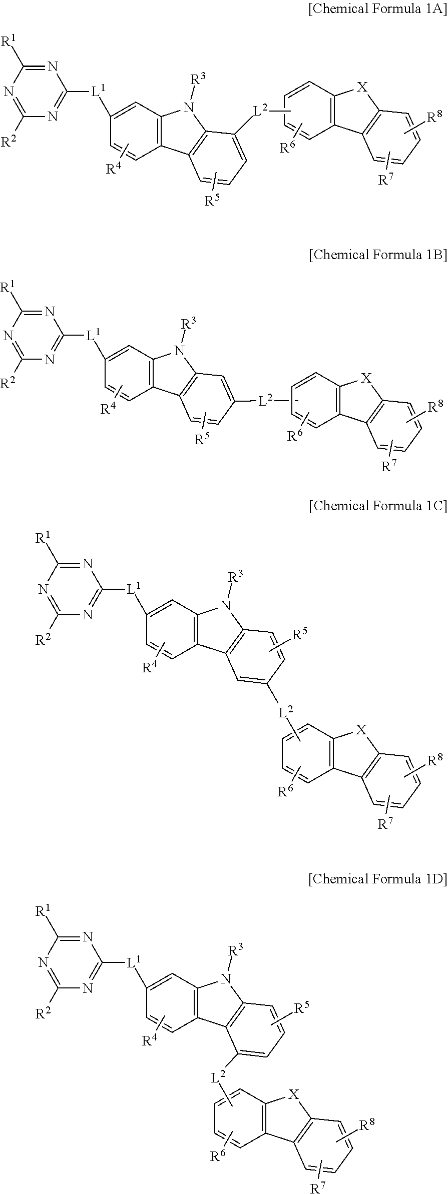
wherein, in Chemical Formula 1A to Chemical Formula 1D, X, L¹, L², and R¹ to R⁸ are defined the same as those of Chemical Formula 1.

3. The compound as claimed in claim 2, wherein:
the compound represented by Chemical Formula 1A is represented by one of Chemical Formula 1 Å-1 to Chemical Formula 1 Å-4,
the compound represented by Chemical Formula 1B is represented by one of Chemical Formula 1B-1 to Chemical Formula 1B-4,
the compound represented by Chemical Formula 1C is represented by one of Chemical Formula 1C-1 to Chemical Formula 1C-4, and
the compound represented by Chemical Formula 1D is represented by one of Chemical Formula 1D-1 to Chemical Formula 1D-4: 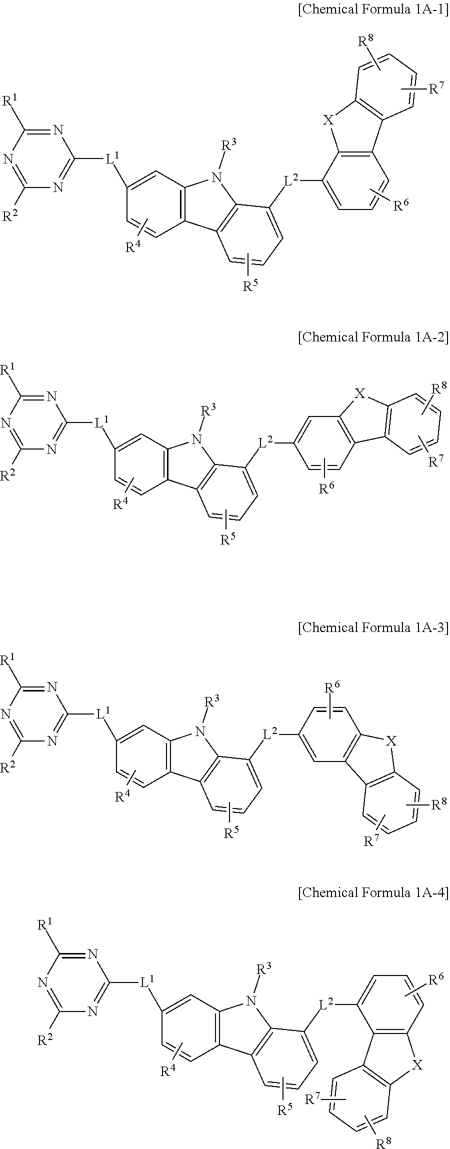 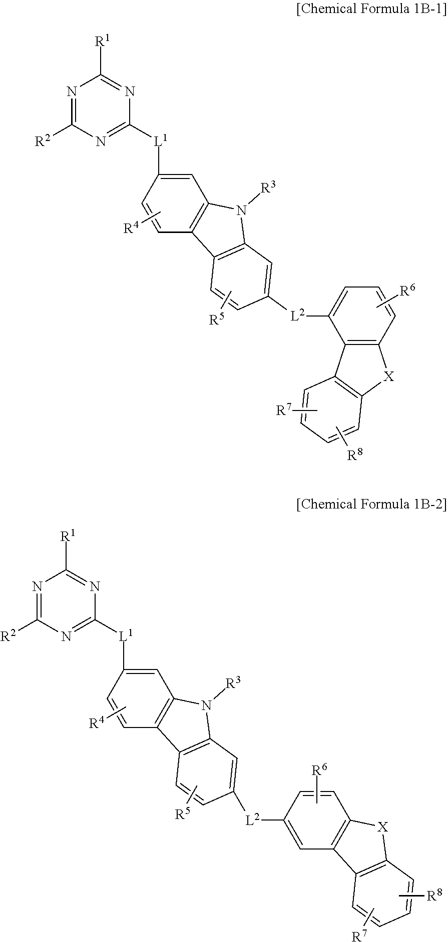 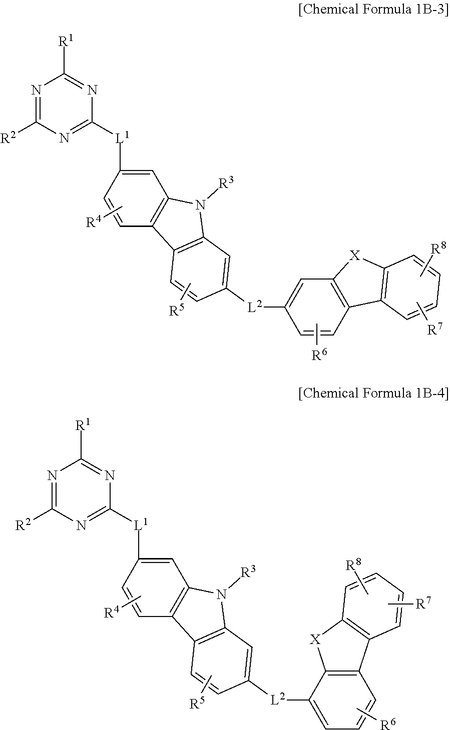 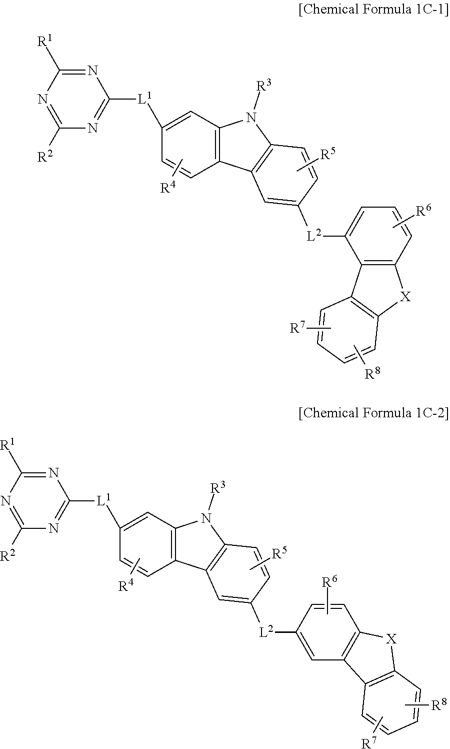 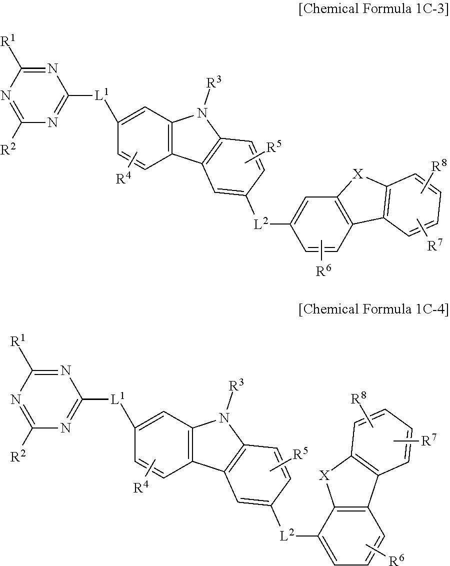 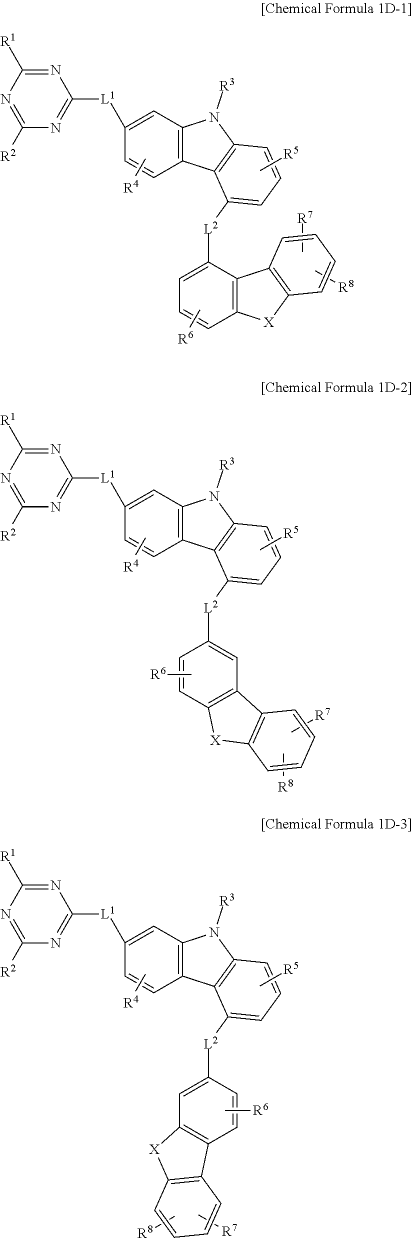 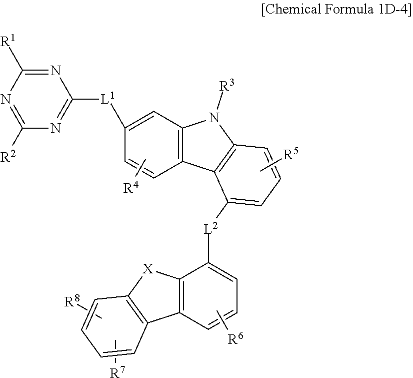
wherein, in Chemical Formula 1 Å-1 to Chemical Formula 1 Å-4, Chemical Formula 1B-1 to Chemical Formula 1B-4 Chemical Formula 1C-1 to Chemical Formula 1C-4, and Chemical Formula 1D-1 to Chemical Formula 1D-4, X, L¹, L², and R¹ to R⁸ are defined the same as those of Chemical Formula 1.

4. The compound as claimed in claim 3, wherein the compound represented by Chemical Formula 1 is represented by Chemical Formula 1C-2.

5. The compound as claimed in claim 1, wherein:
R¹ and R² are independently a substituted or unsubstituted phenyl group, a substituted or unsubstituted para-biphenyl group, or a substituted or unsubstituted meta-biphenyl group,
R³ is a substituted or unsubstituted phenyl group,
R⁴ to R⁸ are independently hydrogen, deuterium, a substituted or unsubstituted C1 to C5 alkyl group, or a substituted or unsubstituted C6 to C12 aryl group, and
L¹ and L² are each a single bond.

6. The compound as claimed in claim 1, wherein the compound represented by Chemical Formula 1 is a compound of the following Group 1:

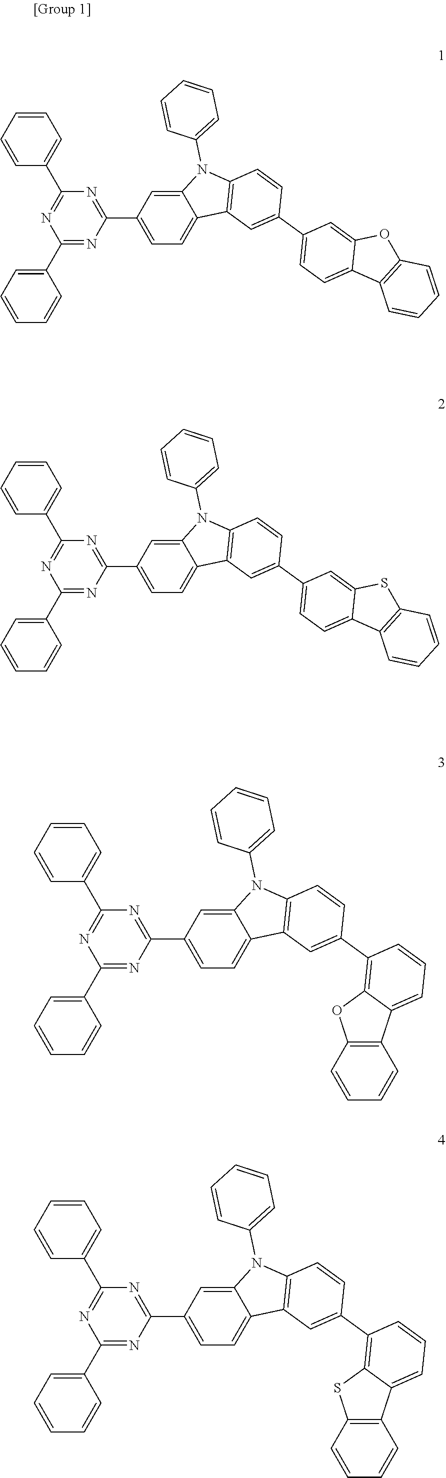 [SMILES: c1ccc(-c2nc(-c3ccccc3)nc(-c3ccc4c5cc(-c6ccc7c(c6)oc6ccccc67)ccc5n(-c5ccccc5)c4c3)n2)cc1 ; c1ccc(-c2nc(-c3ccccc3)nc(-c3ccc4c5cc(-c6ccc7c(c6)sc6ccccc67)ccc5n(-c5ccccc5)c4c3)n2)cc1 ; c1ccc(-c2nc(-c3ccccc3)nc(-c3ccc4c5cc(-c6cccc7c6oc6ccccc67)ccc5n(-c5ccccc5)c4c3)n2)cc1 ; c1ccc(-c2nc(-c3ccccc3)nc(-c3ccc4c5cc(-c6cccc7c6sc6ccccc67)ccc5n(-c5ccccc5)c4c3)n2)cc1]

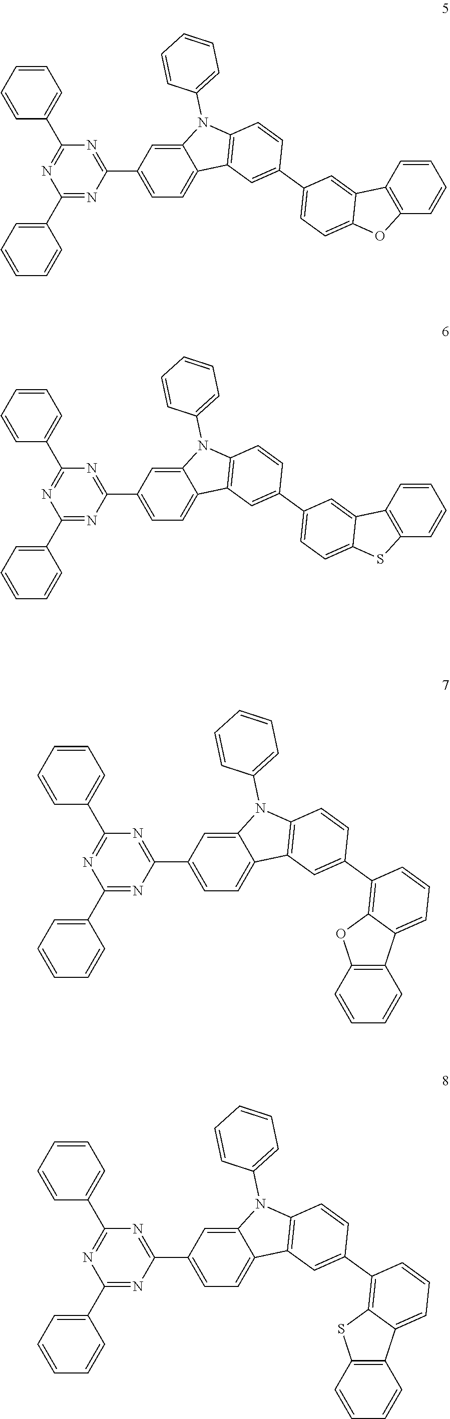 [SMILES: c1ccc(-c2nc(-c3ccccc3)nc(-c3ccc4c5cc(-c6ccc7oc8ccccc8c7c6)ccc5n(-c5ccccc5)c4c3)n2)cc1 ; c1ccc(-c2nc(-c3ccccc3)nc(-c3ccc4c5cc(-c6ccc7sc8ccccc8c7c6)ccc5n(-c5ccccc5)c4c3)n2)cc1 ; c1ccc(-c2nc(-c3ccccc3)nc(-c3ccc4c5cc(-c6cccc7c6oc6ccccc67)ccc5n(-c5ccccc5)c4c3)n2)cc1 ; c1ccc(-c2nc(-c3ccccc3)nc(-c3ccc4c5cc(-c6cccc7c6sc6ccccc67)ccc5n(-c5ccccc5)c4c3)n2)cc1]

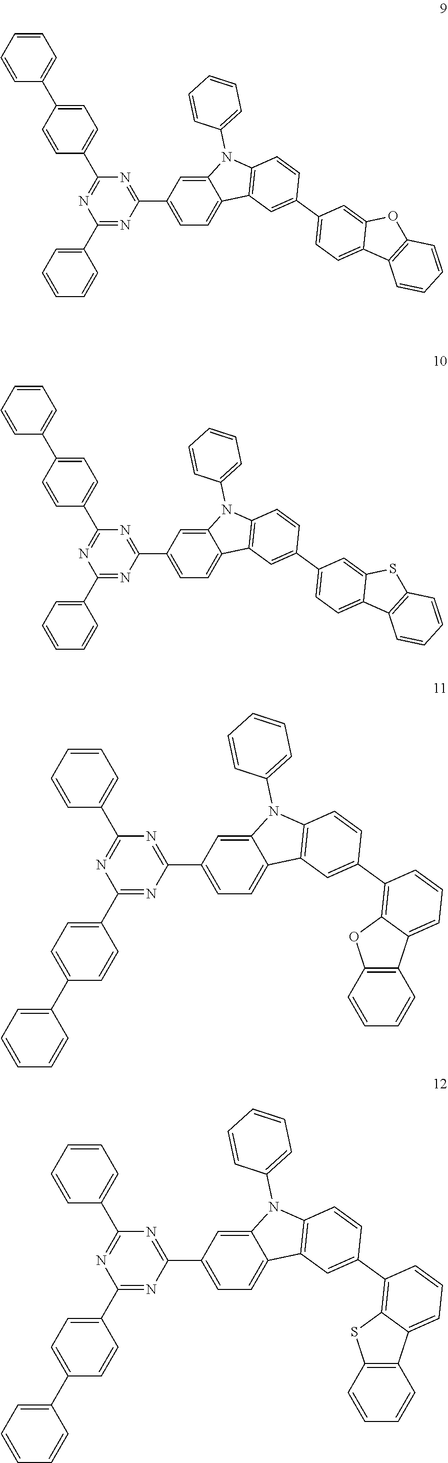 [SMILES: c1ccc(-c2ccc(-c3nc(-c4ccccc4)nc(-c4ccc5c6cc(-c7ccc8c(c7)oc7ccccc78)ccc6n(-c6ccccc6)c5c4)n3)cc2)cc1 ; c1ccc(-c2ccc(-c3nc(-c4ccccc4)nc(-c4ccc5c6cc(-c7ccc8c(c7)sc7ccccc78)ccc6n(-c6ccccc6)c5c4)n3)cc2)cc1 ; c1ccc(-c2ccc(-c3nc(-c4ccccc4)nc(-c4ccc5c6cc(-c7cccc8c7oc7ccccc78)ccc6n(-c6ccccc6)c5c4)n3)cc2)cc1 ; c1ccc(-c2ccc(-c3nc(-c4ccccc4)nc(-c4ccc5c6cc(-c7cccc8c7sc7ccccc78)ccc6n(-c6ccccc6)c5c4)n3)cc2)cc1]

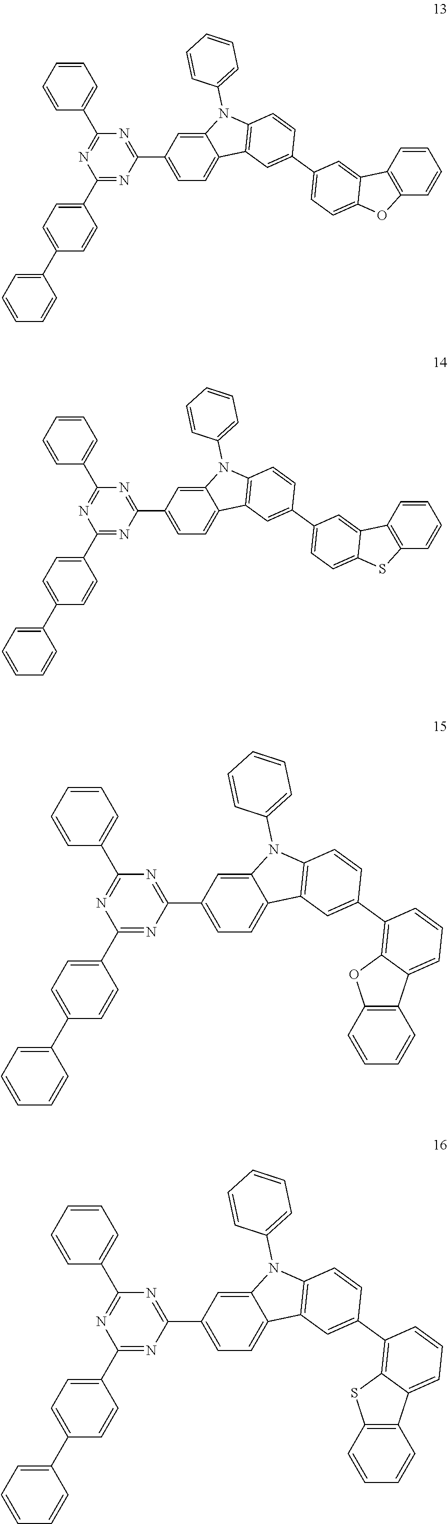 [SMILES: c1ccc(-c2ccc(-c3nc(-c4ccccc4)nc(-c4ccc5c6cc(-c7ccc8oc9ccccc9c8c7)ccc6n(-c6ccccc6)c5c4)n3)cc2)cc1 ; c1ccc(-c2ccc(-c3nc(-c4ccccc4)nc(-c4ccc5c6cc(-c7ccc8sc9ccccc9c8c7)ccc6n(-c6ccccc6)c5c4)n3)cc2)cc1 ; c1ccc(-c2ccc(-c3nc(-c4ccccc4)nc(-c4ccc5c6cc(-c7cccc8c7oc7ccccc78)ccc6n(-c6ccccc6)c5c4)n3)cc2)cc1 ; c1ccc(-c2ccc(-c3nc(-c4ccccc4)nc(-c4ccc5c6cc(-c7cccc8c7sc7ccccc78)ccc6n(-c6ccccc6)c5c4)n3)cc2)cc1]

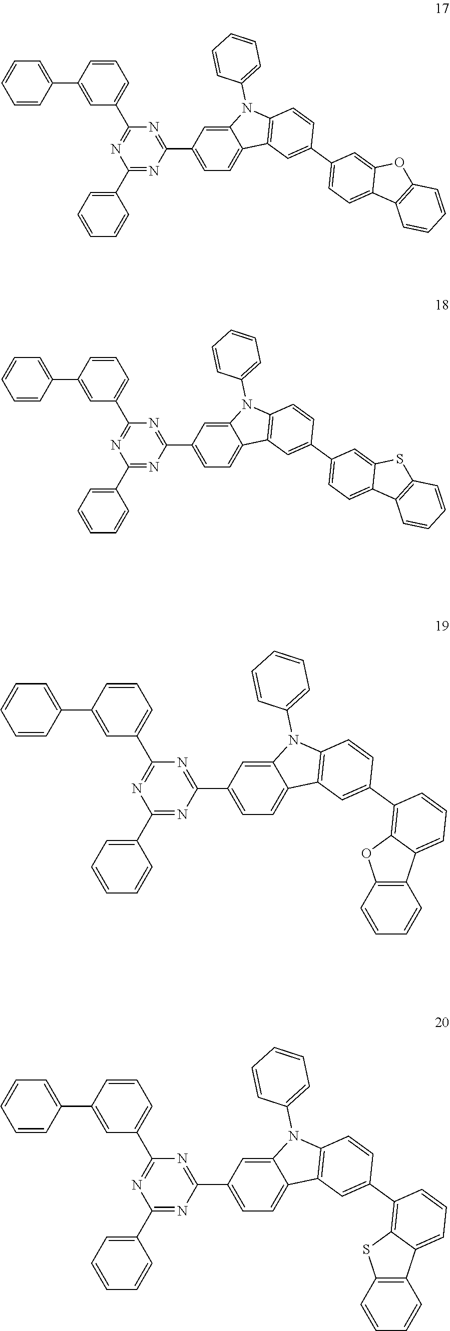 [SMILES: c1ccc(-c2cccc(-c3nc(-c4ccccc4)nc(-c4ccc5c6cc(-c7ccc8c(c7)oc7ccccc78)ccc6n(-c6ccccc6)c5c4)n3)c2)cc1 ; c1ccc(-c2cccc(-c3nc(-c4ccccc4)nc(-c4ccc5c6cc(-c7ccc8c(c7)sc7ccccc78)ccc6n(-c6ccccc6)c5c4)n3)c2)cc1 ; c1ccc(-c2cccc(-c3nc(-c4ccccc4)nc(-c4ccc5c6cc(-c7cccc8c7oc7ccccc78)ccc6n(-c6ccccc6)c5c4)n3)c2)cc1 ; c1ccc(-c2cccc(-c3nc(-c4ccccc4)nc(-c4ccc5c6cc(-c7cccc8c7sc7ccccc78)ccc6n(-c6ccccc6)c5c4)n3)c2)cc1]

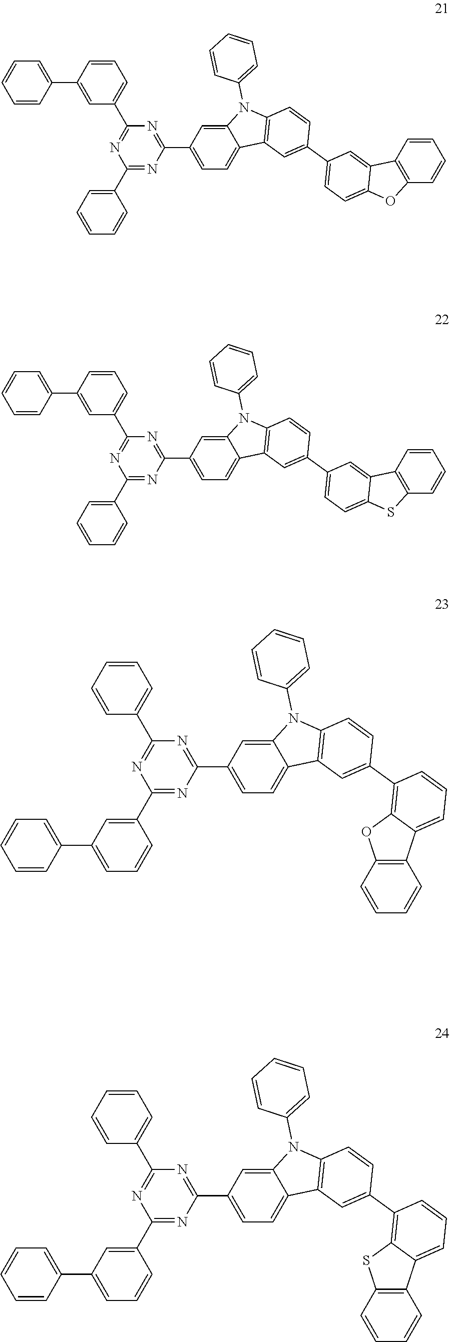 [SMILES: c1ccc(-c2cccc(-c3nc(-c4ccccc4)nc(-c4ccc5c6cc(-c7ccc8oc9ccccc9c8c7)ccc6n(-c6ccccc6)c5c4)n3)c2)cc1 ; c1ccc(-c2cccc(-c3nc(-c4ccccc4)nc(-c4ccc5c6cc(-c7ccc8sc9ccccc9c8c7)ccc6n(-c6ccccc6)c5c4)n3)c2)cc1 ; c1ccc(-c2cccc(-c3nc(-c4ccccc4)nc(-c4ccc5c6cc(-c7cccc8c7oc7ccccc78)ccc6n(-c6ccccc6)c5c4)n3)c2)cc1 ; c1ccc(-c2cccc(-c3nc(-c4ccccc4)nc(-c4ccc5c6cc(-c7cccc8c7sc7ccccc78)ccc6n(-c6ccccc6)c5c4)n3)c2)cc1]

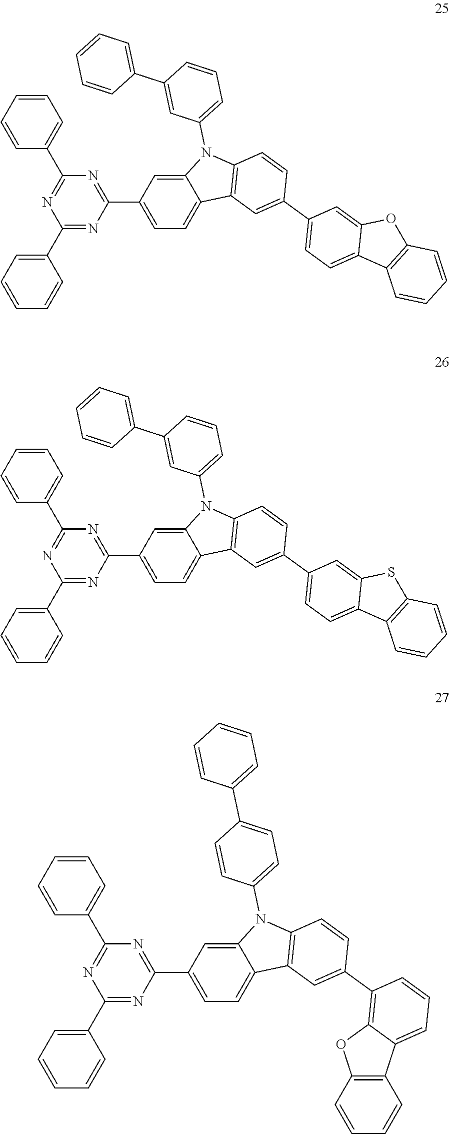 [SMILES: c1ccc(-c2cccc(-n3c4ccc(-c5ccc6c(c5)oc5ccccc56)cc4c4ccc(-c5nc(-c6ccccc6)nc(-c6ccccc6)n5)cc43)c2)cc1 ; c1ccc(-c2cccc(-n3c4ccc(-c5ccc6c(c5)sc5ccccc56)cc4c4ccc(-c5nc(-c6ccccc6)nc(-c6ccccc6)n5)cc43)c2)cc1 ; c1ccc(-c2ccc(-n3c4ccc(-c5cccc6c5oc5ccccc56)cc4c4ccc(-c5nc(-c6ccccc6)nc(-c6ccccc6)n5)cc43)cc2)cc1]

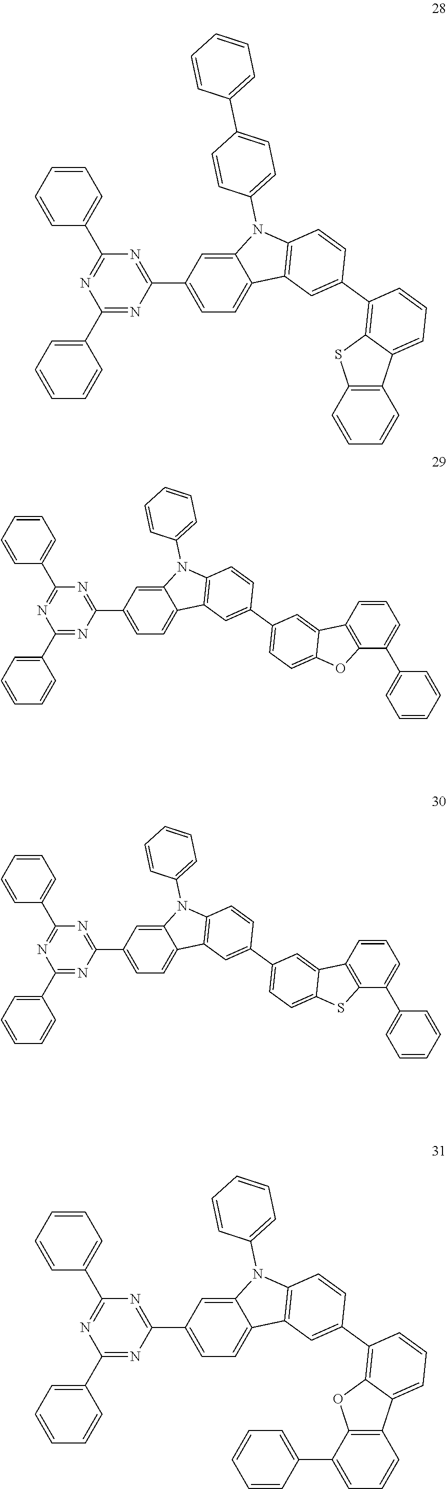 [SMILES: c1ccc(-c2ccc(-n3c4ccc(-c5cccc6c5sc5ccccc56)cc4c4ccc(-c5nc(-c6ccccc6)nc(-c6ccccc6)n5)cc43)cc2)cc1 ; c1ccc(-c2nc(-c3ccccc3)nc(-c3ccc4c5cc(-c6ccc7oc8c(-c9ccccc9)cccc8c7c6)ccc5n(-c5ccccc5)c4c3)n2)cc1 ; c1ccc(-c2nc(-c3ccccc3)nc(-c3ccc4c5cc(-c6ccc7sc8c(-c9ccccc9)cccc8c7c6)ccc5n(-c5ccccc5)c4c3)n2)cc1 ; c1ccc(-c2nc(-c3ccccc3)nc(-c3ccc4c5cc(-c6cccc7c6oc6c(-c8ccccc8)cccc67)ccc5n(-c5ccccc5)c4c3)n2)cc1]

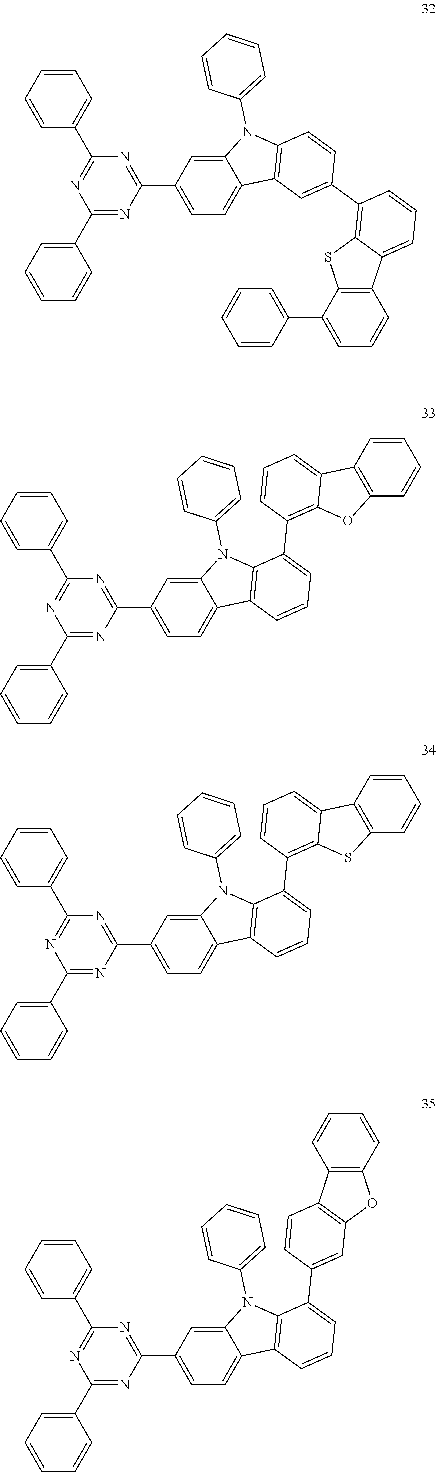 [SMILES: c1ccc(-c2nc(-c3ccccc3)nc(-c3ccc4c5cc(-c6cccc7c6sc6c(-c8ccccc8)cccc67)ccc5n(-c5ccccc5)c4c3)n2)cc1 ; c1ccc(-c2nc(-c3ccccc3)nc(-c3ccc4c5cccc(-c6cccc7c6oc6ccccc67)c5n(-c5ccccc5)c4c3)n2)cc1 ; c1ccc(-c2nc(-c3ccccc3)nc(-c3ccc4c5cccc(-c6cccc7c6sc6ccccc67)c5n(-c5ccccc5)c4c3)n2)cc1 ; c1ccc(-c2nc(-c3ccccc3)nc(-c3ccc4c5cccc(-c6ccc7c(c6)oc6ccccc67)c5n(-c5ccccc5)c4c3)n2)cc1]

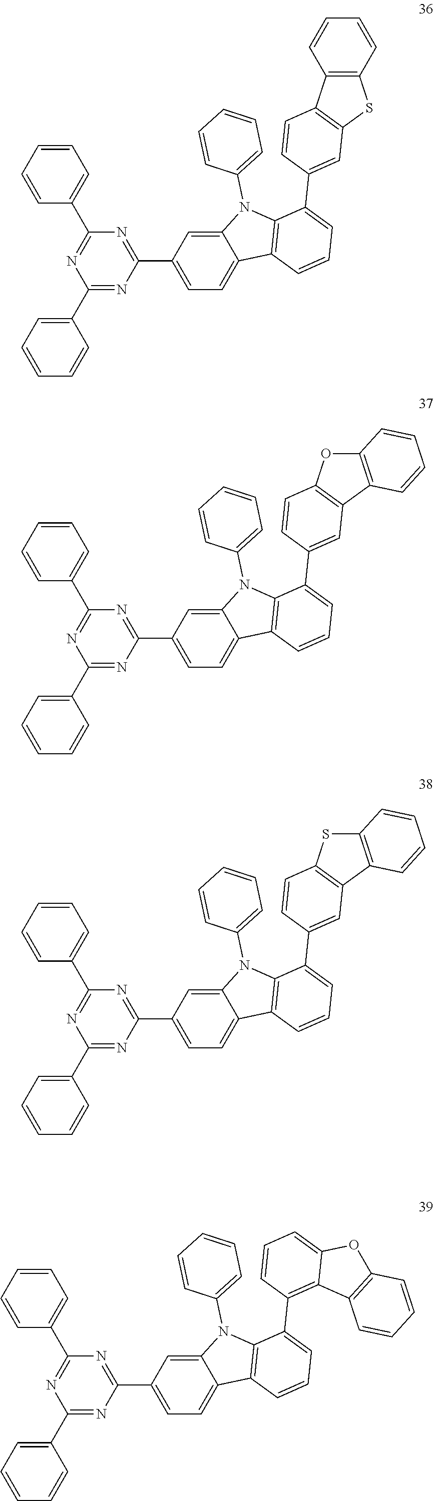 [SMILES: c1ccc(-c2nc(-c3ccccc3)nc(-c3ccc4c5cccc(-c6ccc7c(c6)sc6ccccc67)c5n(-c5ccccc5)c4c3)n2)cc1 ; c1ccc(-c2nc(-c3ccccc3)nc(-c3ccc4c5cccc(-c6ccc7oc8ccccc8c7c6)c5n(-c5ccccc5)c4c3)n2)cc1 ; c1ccc(-c2nc(-c3ccccc3)nc(-c3ccc4c5cccc(-c6ccc7sc8ccccc8c7c6)c5n(-c5ccccc5)c4c3)n2)cc1 ; c1ccc(-c2nc(-c3ccccc3)nc(-c3ccc4c5cccc(-c6cccc7oc8ccccc8c67)c5n(-c5ccccc5)c4c3)n2)cc1]

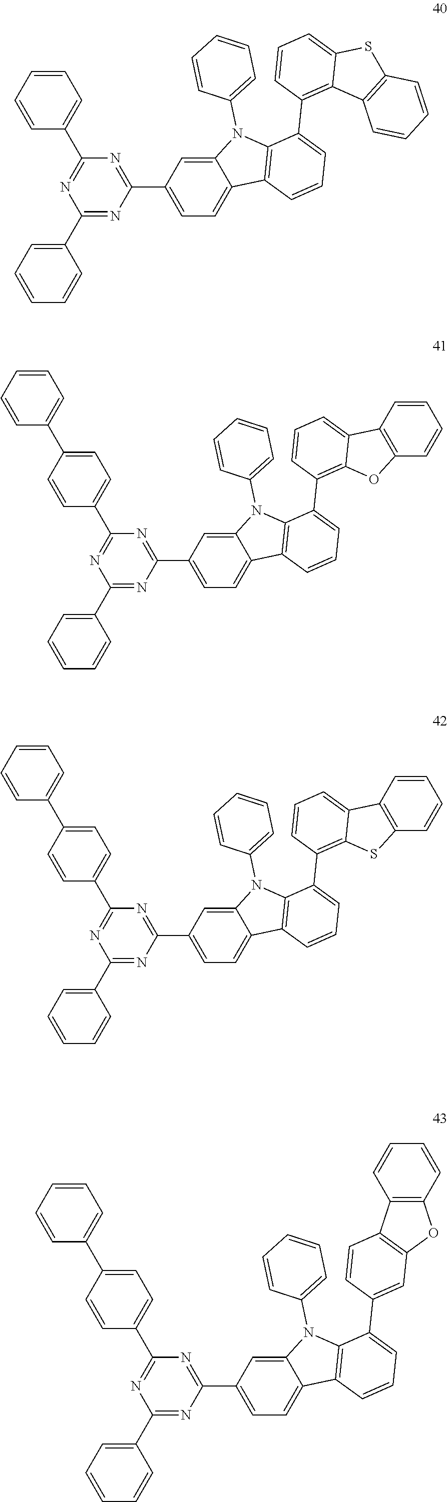 [SMILES: c1ccc(-c2nc(-c3ccccc3)nc(-c3ccc4c5cccc(-c6cccc7sc8ccccc8c67)c5n(-c5ccccc5)c4c3)n2)cc1 ; c1ccc(-c2ccc(-c3nc(-c4ccccc4)nc(-c4ccc5c6cccc(-c7cccc8c7oc7ccccc78)c6n(-c6ccccc6)c5c4)n3)cc2)cc1 ; c1ccc(-c2ccc(-c3nc(-c4ccccc4)nc(-c4ccc5c6cccc(-c7cccc8c7sc7ccccc78)c6n(-c6ccccc6)c5c4)n3)cc2)cc1 ; c1ccc(-c2ccc(-c3nc(-c4ccccc4)nc(-c4ccc5c6cccc(-c7ccc8c(c7)oc7ccccc78)c6n(-c6ccccc6)c5c4)n3)cc2)cc1]

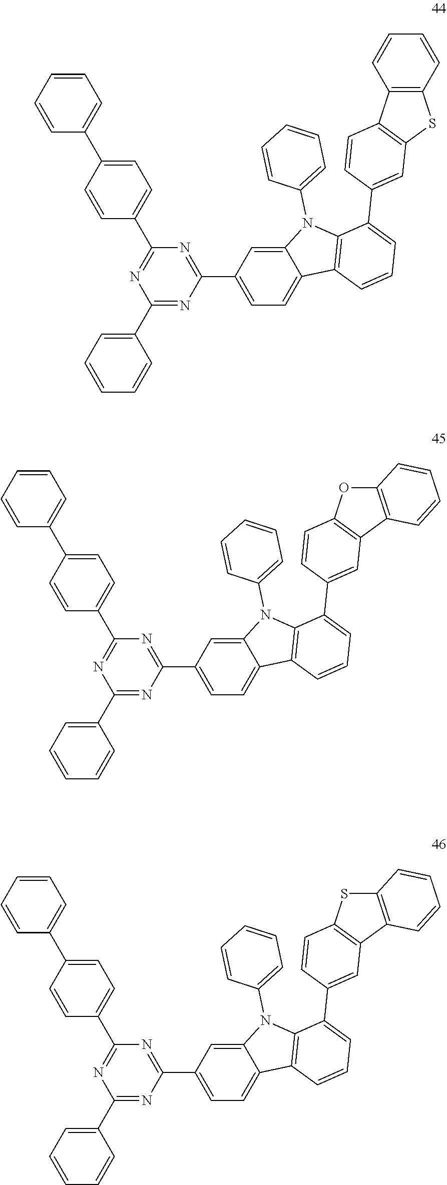 [SMILES: c1ccc(-c2ccc(-c3nc(-c4ccccc4)nc(-c4ccc5c6cccc(-c7ccc8c(c7)sc7ccccc78)c6n(-c6ccccc6)c5c4)n3)cc2)cc1 ; c1ccc(-c2ccc(-c3nc(-c4ccccc4)nc(-c4ccc5c6cccc(-c7ccc8oc9ccccc9c8c7)c6n(-c6ccccc6)c5c4)n3)cc2)cc1 ; c1ccc(-c2ccc(-c3nc(-c4ccccc4)nc(-c4ccc5c6cccc(-c7ccc8sc9ccccc9c8c7)c6n(-c6ccccc6)c5c4)n3)cc2)cc1]

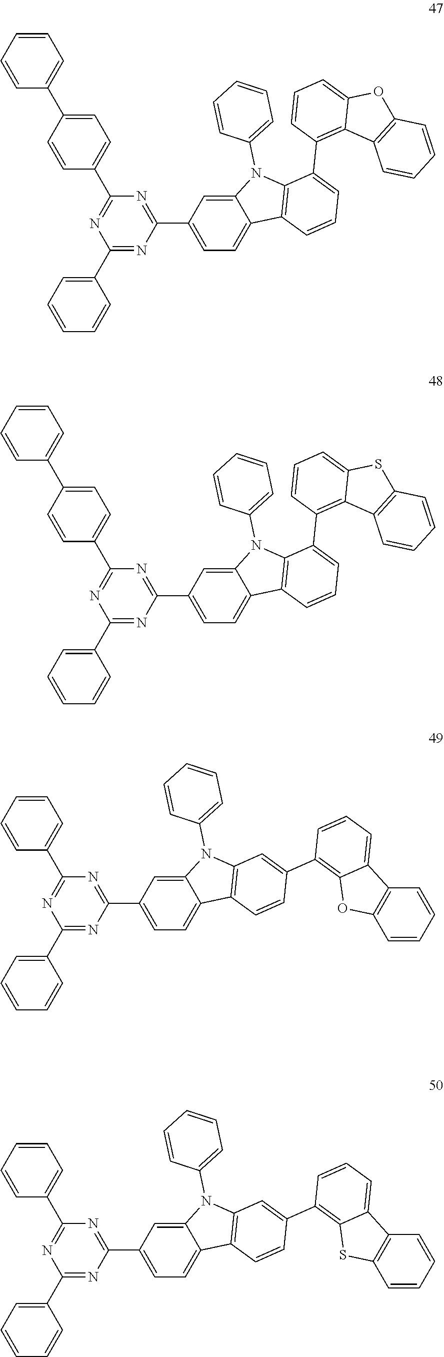 [SMILES: c1ccc(-c2ccc(-c3nc(-c4ccccc4)nc(-c4ccc5c6cccc(-c7cccc8oc9ccccc9c78)c6n(-c6ccccc6)c5c4)n3)cc2)cc1 ; c1ccc(-c2ccc(-c3nc(-c4ccccc4)nc(-c4ccc5c6cccc(-c7cccc8sc9ccccc9c78)c6n(-c6ccccc6)c5c4)n3)cc2)cc1 ; c1ccc(-c2nc(-c3ccccc3)nc(-c3ccc4c5ccc(-c6cccc7c6oc6ccccc67)cc5n(-c5ccccc5)c4c3)n2)cc1 ; c1ccc(-c2nc(-c3ccccc3)nc(-c3ccc4c5ccc(-c6cccc7c6sc6ccccc67)cc5n(-c5ccccc5)c4c3)n2)cc1]

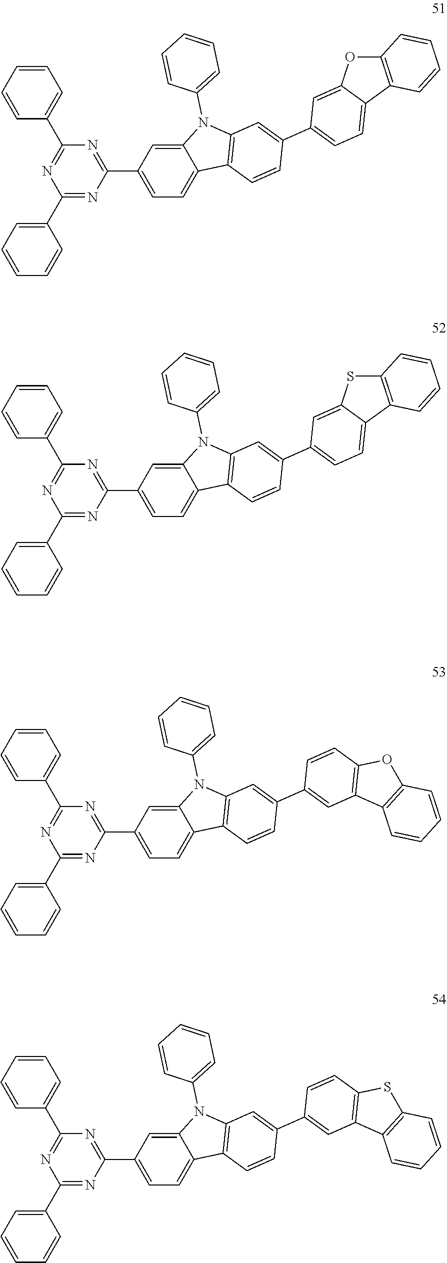 [SMILES: c1ccc(-c2nc(-c3ccccc3)nc(-c3ccc4c5ccc(-c6ccc7c(c6)oc6ccccc67)cc5n(-c5ccccc5)c4c3)n2)cc1 ; c1ccc(-c2nc(-c3ccccc3)nc(-c3ccc4c5ccc(-c6ccc7c(c6)sc6ccccc67)cc5n(-c5ccccc5)c4c3)n2)cc1 ; c1ccc(-c2nc(-c3ccccc3)nc(-c3ccc4c5ccc(-c6ccc7oc8ccccc8c7c6)cc5n(-c5ccccc5)c4c3)n2)cc1 ; c1ccc(-c2nc(-c3ccccc3)nc(-c3ccc4c5ccc(-c6ccc7sc8ccccc8c7c6)cc5n(-c5ccccc5)c4c3)n2)cc1]

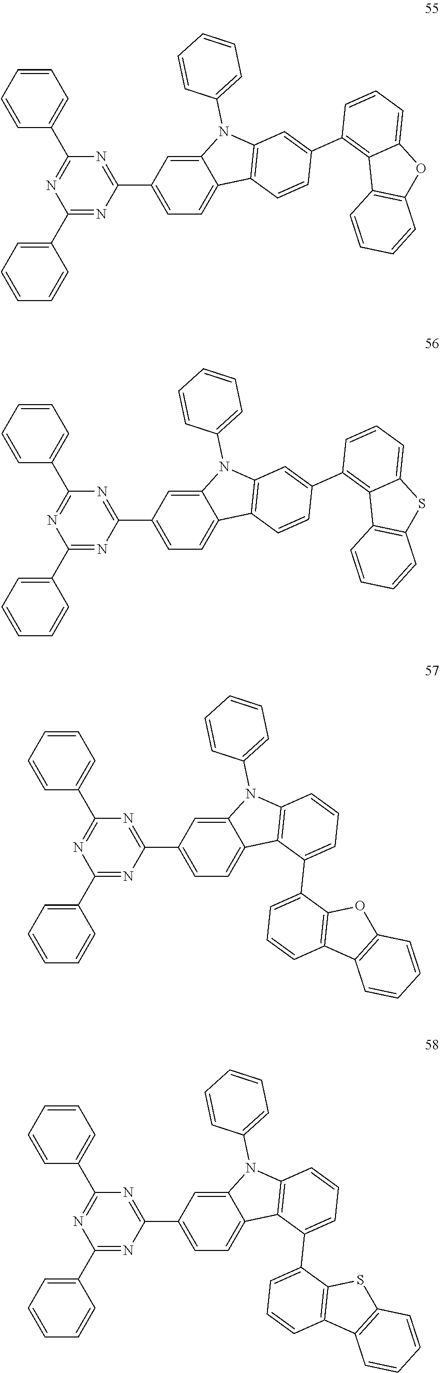 [SMILES: c1ccc(-c2nc(-c3ccccc3)nc(-c3ccc4c5ccc(-c6cccc7oc8ccccc8c67)cc5n(-c5ccccc5)c4c3)n2)cc1 ; c1ccc(-c2nc(-c3ccccc3)nc(-c3ccc4c5ccc(-c6cccc7sc8ccccc8c67)cc5n(-c5ccccc5)c4c3)n2)cc1 ; c1ccc(-c2nc(-c3ccccc3)nc(-c3ccc4c5c(-c6cccc7c6oc6ccccc67)cccc5n(-c5ccccc5)c4c3)n2)cc1 ; c1ccc(-c2nc(-c3ccccc3)nc(-c3ccc4c5c(-c6cccc7c6sc6ccccc67)cccc5n(-c5ccccc5)c4c3)n2)cc1]

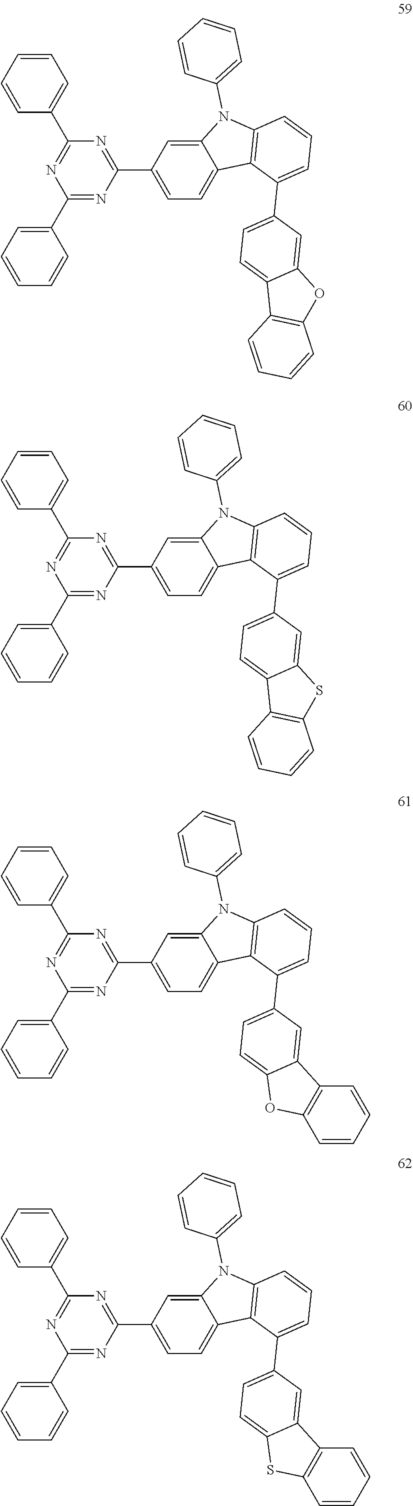 [SMILES: c1ccc(-c2nc(-c3ccccc3)nc(-c3ccc4c5c(-c6ccc7c(c6)oc6ccccc67)cccc5n(-c5ccccc5)c4c3)n2)cc1 ; c1ccc(-c2nc(-c3ccccc3)nc(-c3ccc4c5c(-c6ccc7c(c6)sc6ccccc67)cccc5n(-c5ccccc5)c4c3)n2)cc1 ; c1ccc(-c2nc(-c3ccccc3)nc(-c3ccc4c5c(-c6ccc7oc8ccccc8c7c6)cccc5n(-c5ccccc5)c4c3)n2)cc1 ; c1ccc(-c2nc(-c3ccccc3)nc(-c3ccc4c5c(-c6ccc7sc8ccccc8c7c6)cccc5n(-c5ccccc5)c4c3)n2)cc1]

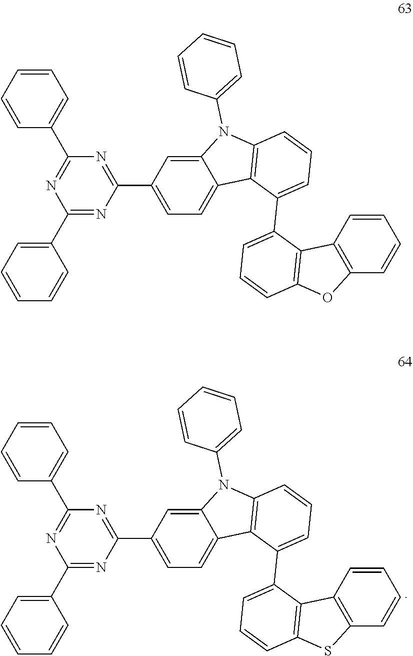 [SMILES: c1ccc(-c2nc(-c3ccccc3)nc(-c3ccc4c5c(-c6cccc7oc8ccccc8c67)cccc5n(-c5ccccc5)c4c3)n2)cc1 ; c1ccc(-c2nc(-c3ccccc3)nc(-c3ccc4c5c(-c6cccc7sc8ccccc8c67)cccc5n(-c5ccccc5)c4c3)n2)cc1]

7. A composition for an organic optoelectronic device, the composition comprising:
a first compound for an organic optoelectronic device and a second compound for an organic optoelectronic device,
wherein:
the first compound is the compound as claimed in claim 1, and
the second compound is represented by Chemical Formula 2; or a combination of Chemical Formula 3 and Chemical Formula 4: 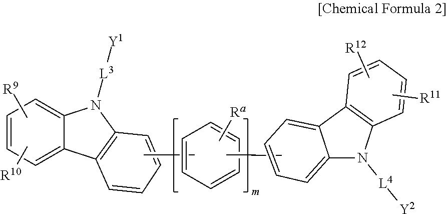
in Chemical Formula 2,
Y¹ and Y² are independently a substituted or unsubstituted C6 to C20 aryl group, or a substituted or unsubstituted C2 to C30 heterocyclic group,
L³ and L⁴ are independently a single bond or a substituted or unsubstituted C6 to C20 arylene group,
R^{a} and R⁹ to R¹² are independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, and
m is an integer of 0 to 2; 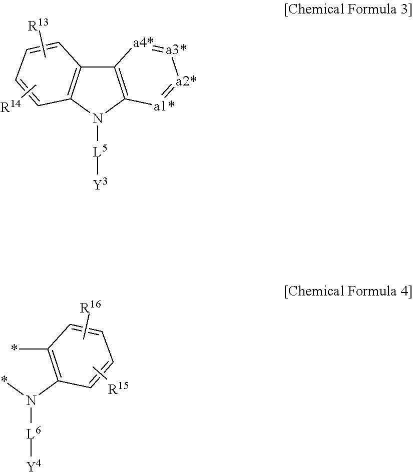
in Chemical Formulas 3 and 4,
Y³ and Y⁴ are independently a substituted or unsubstituted C6 to C20 aryl group or a substituted or unsubstituted C2 to C30 heterocyclic group,
an adjacent two of a1* to a4* are linking carbons linked at * of Chemical Formula 4 and the other two of a1* to a4* are C-L^{a}-R^{b},
L^{a}, L⁵, and L⁶ are independently a single bond or a substituted or unsubstituted C6 to C20 arylene group, and
R^{b} and R¹³ to R¹⁶ are independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group.

8. An organic optoelectronic device, comprising:
an anode and a cathode facing each other, and
at least one organic layer between the anode and the cathode,
wherein the at least one organic layer includes the compound as claimed in claim 1.

9. The organic optoelectronic device as claimed in claim 8, wherein:
the at least one organic layer includes a light emitting layer, and
the light emitting layer includes the compound.

10. A display device comprising the organic optoelectronic device as claimed in claim 8.

11. An organic optoelectronic device, comprising:
an anode and a cathode facing each other, and
at least one organic layer between the anode and the cathode,
wherein at least one organic layer includes the composition as claimed in claim 7.

12. The organic optoelectronic device as claimed in claim 11, wherein:
the at least one organic layer includes a light emitting layer, and
the light emitting layer includes the composition.

13. A display device comprising the organic optoelectronic device as claimed in claim 11.
